(19) United States
(12) Patent Application Publication
(10) Pub. No.: US 2005/0038098 A1
(43) Pub. Date: Feb. 17, 2005

(54) Substituted dihydronaphthalene and isochroman compounds for the treatment of metabolic disorders, cancer and other diseases

(76) Inventors: Catherine Tachdjian, San Diego, CA (US); Jianhua Guo, San Diego, CA (US); Mohamed Boudjelal, San Diego, CA (US); Hussien A. Al-Shamma, Encinitas, CA (US); Andrea Fanjul Giachino, San Diego, CA (US); Karine Jakubowicz-Jaillardon, Villebon sur Yvette (FR); Qing Chen, San Diego, CA (US); James W. Zapf, San Diego, CA (US); Magnus Pfahl, Solana Beach, CA (US)

Correspondence Address: NEEDLE & ROSENBERG, P.C., SUITE 1000, 999 PEACHTREE STREET, ATLANTA, GA, 30309-3915, US

(21) Appl. No.: 10/827,111

(22) Filed: Apr. 19, 2004

Related U.S. Application Data
(60) Provisional application No. 60/464,388, filed on Apr. 18, 2003.

(51) Int. Cl. C07D 49/02; C07D 45/02; A61K 31/416; A61K 31/4025; A61K 31/381; A61K 31/353

(52) U.S. Cl. 514/406; 514/422; 514/444; 514/456; 548/364.4; 548/525; 549/60; 549/403

(57) ABSTRACT

The invention relates to novel heterocyclic compounds having the structure illustrated by Formula (I) 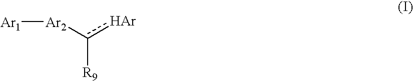
wherein the Ar₁ radicals are substituted dihydronapthalene or isochroman radicals, the Ar₂ radicals are aryl or heteroaryl radicals; and HAr is a 2,4-thiazolidinedione, 2-thioxo-thiazolidine-4-one, 2,4-imidazolidinedione or 2-thioxo-imidazolidine-4-one radical. The compounds of Formula (I) can have biological activity for advantageously regulating carbohydrate metabolism, including serum glucose level, and lipid metabolism, and can be useful for the treatment of hyperlipidemia and/or hypercholesterolemia, and Type II diabetes. The compounds of Formula (I) can also have utility in the treatment of diseases of uncontrolled proliferation, including cancer.

### RELATED APPLICATIONS

This application claims priority to the U.S. Provisional Application Ser. No. 60/464,388, filed Apr. 18, 2003, the entire disclosure of which application is hereby incorporated herein in its entirety by this reference.

### BACKGROUND OF THE INVENTION

Type 2 diabetes, also referred to as non-insulin dependent diabetes mellitus (NIDDM), afflicts between 80 and 90% of all diabetic patients in developed countries. In the United States alone, approximately 15 million people, and more than 100 million worldwide, are affected. Because this disorder is a late onset disease and occurs often in overweight persons, it can be expected that the number of patients suffering from this disease will increase further. Patients suffering from type 2 diabetes usually still produce insulin but become increasingly resistant to their own insulin and to insulin therapy.

A new class of drugs has been recently introduced that resensitizes patients to their own insulin (insulin sensitizers), thereby reducing blood glucose levels, and thus abolishing, or at least reducing, the requirement for exogenous insulin. Troglitazone (Resulin™) and rosiglitazone (Avandia™) were among the first representatives of this class of drugs approved for the treatment of type 2 diabetes in the United States and several other countries. The currently approved compounds can however have side effects including rare but severe liver toxicities and they can increase body weight in humans. Such side effects are of major concern for diabetes patients who can require treatment for a decade or longer. Therefore, new and better drugs for the treatment of type 2 diabetes and related disorders are needed. In particular, drugs that can control blood sugar levels and simultaneously control hyperlipidemia and/or hypercholesterolemia are desirable. Elevated levels of cholesterol lead to atherosclerosis and heart disease which in many type 2 diabetes patients is the cause of death.

There is also a need for the more effective drugs to treat diseases of uncontrolled cellular proliferation, such as cancers. Molecules that have strong cellular differentiation activity can inhibit the uncontrolled cellular proliferation of cancer cells, in particular breast cancer.

Small molecules that can be effective for the treatment of diabetes and/or disorders of carbohydrate metabolism were disclosed in U.S. Pat. No. 6,515,003, issued Feb. 4, 2003, based on U.S. patent application Ser. No. 09/652,810, filed Aug. 31, 2000, which claimed priority to U.S. Provisional Patent Application 60/151,670, filed Aug. 31, 1999. Small molecules that can be useful in the treatment of certain cancers were disclosed in PCT Patent Application WO 01/16122, published Mar. 8, 2001, which claimed priority to the same U.S. Provisional Patent Application 60/151,670 cited above. The disclosures of all the above-described patent documents are hereby incorporated herein by this reference, for both their chemical structural disclosures, their teachings of the biological activities of those compounds, methods of making the compounds, and methods for their use as pharmaceutical compositions.

There is however a continuing need for effective drugs for the treatment of cancers, especially breast cancer, and for the treatment of type 2 diabetes and associated disorders of carbohydrate and/or lipid metabolism, including hyperlipidemia and hypercholesterolemia. In particular, there is a continuing need for new drugs that can control the blood sugar levels of diabetics, and simultaneously control hyperlipidemia and hypercholesterolemia so as to lessen or prevent atherosclerosis.

### SUMMARY OF THE INVENTION

Some embodiments of the invention relate to novel heterocyclic compounds having the structure illustrated by Formula (I)

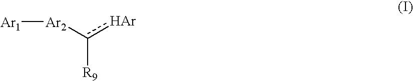

The compounds of Formula (I) comprise Ar₁ radicals that are substituted dihydronapthalene or isochroman radicals. The Ar₂ radicals are aryl or heteroaryl radicals that include “meta”-substituted benzene, pyridine, pyrimidine, pyrazine, thiofuran, furan, pyrrole, or pyrazole radicals; - - - indicates that a second carbon-carbon double bond is either present or absent; R₉ is hydrogen, hydroxy, or an alkyl radical; and HAr is a 2,4-thiazolidinedione, 2-thioxo-thiazolidine-4-one, 2,4-imidazolidinedione or 2-thioxo-imidazolidine-4-one radical. The inventions also relate to pharmaceutically acceptable salts of the compounds of Formula (I).

The compounds of Formula (I) have been found to have biological activity for advantageously regulating carbohydrate metabolism, including serum glucose levels. The compounds of Formula (I) have also been found to be biologically active as modulators of lipid metabolism, and are therefore useful for the treatment of hyperlipidemia and/or hypercholesterolemia. Therefore, the compounds of Formula (I) can simultaneously and beneficially regulate carbohydrate and lipid metabolism so as to simultaneously decrease levels of serum glucose, serum triglycerides, and/or serum cholesterol. The compounds of Formula (I) have also been found to have unexpectedly superior pharmaceutical physical properties, including unexpectedly superior oral bioavailability, as compared to prior art compounds. As a result of their combination of good biological activity and superior physical properties and bioavailability, it has been found that the compounds of Formula (I) can be unexpectedly superior for the treatment of type 2 diabetes and the simultaneous treatment of the hyperlipidemia, hypercholesterolemia, and/or atherosclerosis which is often associated with diabetes.

The compounds of Formula (I) also show activity for inducing differentiation in certain well known cell lines of pre-adipocytes. The ability of a compound to induce differentiation of these cell lines can correlate with insulin sensitizing and lipid lowering or lipid modulating activities. The adipocyte differentiation activity can also correlate with anticancer activity. Therefore, the compounds of Formula (I) have utility in the treatment of diseases of uncontrolled proliferation. The compounds of Formula (I) have shown unexpectedly superior results for the treatment of breast cancer in an in vivo rat model of breast cancer.

Further embodiments of the compounds of Formula (I), and pharmaceutical compositions comprising one or more of the compounds of Formula (I) will be described in more detail in the specification and written description hereinbelow. Other embodiments of the invention relate to methods of synthesizing the compounds of Formula (I).

The invention also provides methods for the treatment of diabetes and associated diseases, and regulating carbohydrate and/or lipid metabolism, as well as methods for the treatment of diseases of uncontrolled cellular proliferation comprising administering to a mammal diagnosed as having one of the cited diseases or metabolic disorders, one or more compounds of Formula (I), or a pharmaceutical composition thereof, which may contain additional components that are pharmaceutically active to treat the relevant diseases. The compounds of Formula (I) also have a combination of high biological activities, bioavailabilities, and physical properties that can provide unexpectedly superior properties when formulated, so as to provide improved pharmaceutical compositions.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention.

### BRIEF DESCRIPTION OF THE DRAWINGS

FIGS. 1a-b show the results of in-vitro screening assays for the ability of some of the isochroman and dihydronaphthalene compounds of Formula (I) to induce differentiation of 3T3-L1pre-adipocytes to adipocytes.

FIGS. 2a-d show the ability of certain isochroman and dihydronaphthalene compounds of Formula (I), when orally administered, to simultaneously decrease the serum glucose and triglyceride levels of KKA^{y} mice, as compared to control KKA^{y} mice that did not receive the compounds.

FIG. 3 shows the ability of isochroman compound 11 to increase cholesterol efflux from macrophage cells in vitro.

FIG. 4a-b illustrate the ability of a dihydronapthalene compound of Formula (I) to decrease total cholesterol and LDL (bad cholesterol) while increasing HDL (good cholesterol) in Sprague Dawley rats.

FIG. 5 shows the ability of various isochroman, and dihydronaphthalene, compounds of Formula (I) to down regulate Cyclin D1 expression in MCF-7 breast cancer cells in vitro.

FIG. 6 shows the ability of various isochroman, dihydronaphthalene, and tetrahydroquinoline compounds of Formula (I) to decrease the number of progressing carcinogen induced mammary tumors in Sprague Dawley rats, and increase the number of static and regressing tumors.

FIG. 7 illustrates several overall synthetic strategies for synthesizing compounds of Formulas (Iₐ) and (I_{b}).

FIG. 8 shows methods for synthesizing precursors isochroman radicals of Formulas (Ar₁ₑ) and (Ar_{1f}).

FIG. 9 shows methods for further functionalizing precursors of isochroman radicals (Ar₁ₑ) and (Ar_{1f})

FIG. 10 shows methods for synthesizing precursors of dihydronapthalenyl radicals of Formula (Ar_{1g}) and (Ar₁ₕ).

### DETAILED DESCRIPTION

The present invention can be understood more readily by reference to the following detailed description of various embodiments of the invention and the Examples included therein and to the Figures and their previous and following description. Before the present compounds, compositions, and/or methods are disclosed and described, it is to be understood that this invention is not limited to specific synthetic methods, specific pharmaceutical carriers or formulations, or to particular modes of administering the compounds of the invention, as such can, of course, vary. It is also to be understood that the terminology used herein is for the purpose of describing particular embodiments only and is not intended to be limiting.

The present invention relates to compounds, such as those of Formula (I) that are useful, for example, to modulate lipid and/or carbohydrate metabolism, and especially for the treatment of diabetes, such as type 2 diabetes, and other diseases. In addition, compounds of the invention have demonstrated unexpectedly superior oral bioavailability, as exhibited by their high blood levels after oral dosing in animals. Oral bioavailability allows oral dosing for use in chronic diseases, with the advantage of self-administration and decreased cost over other means of administration. The compounds described herein can be used effectively to prevent, alleviate or otherwise treat type 2 diabetes and/or other disease states in mammals and/or humans, such as atherosclerosis and diseases related to inflammation and/or uncontrolled proliferation, including cancers such as breast cancer.

Definitions

In the specification and Formulae described herein the following terms are hereby defined.

“Optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyls where there is substitution.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

By the term “effective amount” of a compound as provided herein is meant a sufficient amount of the compound to provide the desired regulation of a desired function, such as gene expression, protein function, or a disease condition. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “alkyl” denotes a hydrocarbon group or residue which is structurally similar to a non-cyclic alkane compound modified by the removal of one hydrogen from the non-cyclic alkane and the substitution therefore of a non-hydrogen group or residue. Alkyls comprise a noncyclic, saturated, straight or branched chain hydrocarbon residue having from 1 to 12 carbons, or 1 to 8 carbons, or 1 to 6 carbons. Examples of such alkyl radicals include methyl, ethyl, n-propyl, iso-propyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms.

The term “substituted alkyl” denotes an alkyl radical analogous to the above definition that is further substituted with one, two, or more additional organic or inorganic substituent groups. Suitable substituent groups include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When more than one substituent group is present then they can be the same or different. The organic substituent groups can comprise from 1 to 12 carbon atoms, or from 1 to 6 carbon atoms, or from 1 to 4 carbon atoms.

The term “alkenyl” denotes an alkyl residue as defined above that comprises at least one carbon-carbon double bond. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “substituted alkenyl” denotes an alkenyl residue as defined above definitions that is substituted with one or more groups, but preferably one, two or three groups, selected from halogen, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “alkynyl” denotes a residue as defined above that comprises at least one carbon-carbon double bond. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “substituted alkynyl” denotes an alkylnyl residue of the above definition that is substituted with one or more groups, but preferably one or two groups, selected from halogen, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “cycloalkyl” denotes a hydrocarbon group or residue which is structurally similar to a cyclic alkane compound modified by the removal of one hydrogen from the cyclic alkane and substitution therefore of a non-hydrogen group or residue. Cycloalkyl groups, or residues radical contain 3 to 18 carbons, or preferably 4 to 12 carbons, or 5 to 8 carbons. Examples include as cyclopropyl, cyclobutyl, cyclopentyl, cyclohexyl, cycloheptyl, decahydronapthyl, adamantyl, and like residues.

The term “substituted cycloalkyl” denotes a cycloalkyl residue as defined above that is further substituted with one, two, or more additional organic or inorganic groups that can include but are not limited to halogen, alkyl, substituted alkyl, hydroxyl, alkoxy, substituted alkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

The term “cycloalkenyl” denotes a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like. The term “substituted cycloalkenyl” denotes a cycloalkyl as defined above further substituted with one or more groups selected from halogen, alkyl, hydroxyl, alkoxy, substituted alkoxy, haloalkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkenyl is substituted with more than one group, they can be the same or different. The organic substituent groups can comprise from 1 to 12 carbon atoms, or from 1 to 6 carbon atoms, or from 1 to 4 carbon atoms.

The term “alkoxy” as used herein denotes an alkyl residue, defined above, attached directly to an oxygen to form an ether residue. Examples include methoxy, ethoxy, n-propoxy, iso-propoxy, n-butoxy, t-butoxy, iso-butoxy and the like.

The term “substituted alkoxy” denotes an alkoxy residue of the above definition that is substituted with one or more substituent groups, but preferably one or two groups, which include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “mono-substituted amino” denotes an amino substituted with one organic substituent groups, which include but are not limited to alkyl, substituted alkyl or arylalkyl wherein the terms have the same definitions found hereinabove. Examples of mono-substituted amino groups include methylamino (—NH—CH₃); ethylamino (—NHCH₂CH₃), hydroxyethylamino (—NH—CH₂CH₂OH), and the like.

The term “di-substituted amino” denotes an amino residue substituted with two organic radicals that can be same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

The term “haloalkyl” denotes an alkyl residue as defined above, substituted with one or more halogens, preferably fluorine, such as a trifluoromethyl, pentafluoroethyl and the like.

The term “haloalkoxy” denotes a haloalkyl residue as defined above that is directly attached to an oxygen to form trifluoromethoxy, pentafluoroethoxy and the like.

The term “acyl” denotes a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

The term “acyloxy” denotes a an acyl radical as defined above directly attached to an oxygen to form an R—C(O)O— residue. Examples include but are not limited to acetyloxy, propionyloxy, butanoyloxy, iso-butanoyloxy, benzoyloxy and the like.

The term “aryl” denotes a ring radical containing 6 to 18 carbons, or preferably 6 to 12 carbons, having at least one six-membered aromatic “benzene” residue therein. Examples of such aryl radicals include phenyl, naphthyl, and ischroman radicals. The term “substituted aryl” denotes an aryl ring radical as defined above that is substituted with one or more, or preferably 1, 2, or 3 organic or inorganic substituent groups, which include but are not limited to a halogen, alkyl, substituted alkyl, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring wherein the terms are defined herein. The organic substituent groups can comprise from 1 to 12 carbon atoms, or from 1 to 6 carbon atoms, or from 1 to 4 carbon atoms.

The term “heteroaryl” denotes an aryl ring radical as defined above, wherein at least one of the carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

The term “halo” or “halogen” refers to a fluoro, chloro, bromo or iodo group.

The term “thioalkyl” denotes a sulfide radical containing 1 to 8 carbons, linear or branched. Examples include methylsulfide, ethyl sulfide, isopropylsulfide and the like.

The term “thiohaloalkyl” denotes a thioalkyl radical substituted with one or more halogens. Examples include trifluoromethylthio, 1,1-difluoroethylthio, 2,2,2-trifluoroethylthio and the like.

The term “carboalkoxy” refers to an alkyl ester of a carboxylic acid, wherein alkyl has the same definition as found above. Examples include carbomethoxy, carboethoxy, carboisopropoxy and the like.

The term “alkylcarboxamido” denotes a single alkyl group attached to the amine of an amide, wherein alkyl has the same definition as found above. Examples include N-methylcarboxamide, N-ethylcarboxamide, N-(iso-propyl)carboxamide and the like. The term “substitutedo” denotes a single “substituted alkyl” group, as defined above, attached to the amine of an amide.

The term “dialkylcarboxamido” denotes two alkyl or arylalkyl groups that are the same or different attached to the amine of an amide, wherein alkyl has the same definition as found above. Examples of a dialkylcarboxamido include N,N-dimethylcarboxamide, N-methyl-N-ethylcarboxamide and the like. The term “substituted dialkylcarboxamido” denotes two alkyl groups attached to the amine of an amide, where one or both groups is a “substituted alkyl”, as defined above. It is understood that these groups can be the same or different. Examples include N,N-dibenzylcarboxamide, N-benzyl-N-methylcarboxamide and the like.

The term “arylalkyl” defines an alkylene, such as —CH₂— for example, which is substituted with an aryl group that can be substituted or unsubstituted as defined above. Examples of an “arylalkyl” include benzyl, phenethylene and the like.

A residue of a chemical species, as used in the specification and concluding claims, refers to a structural fragment, or a moiety that is the resulting product of the chemical species in a particular reaction scheme or subsequent formulation or chemical product, regardless of whether the structural fragment or moiety is actually obtained from the chemical species. Thus, an ethylene glycol residue in a polyester refers to one or more —OCH₂CH₂O— repeat units in the polyester, regardless of whether ethylene glycol is used to prepare the polyester. Similarly, a 2,4-thiazolidinedione residue in a chemical compound refers to one or more -2,4-thiazolidinedione moieties of the compound, regardless of whether the residue was obtained by reacting 2,4-thiazolidinedione to obtain the compound.

The term “organic residue” defines a carbon containing residue, i.e. a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic resides can preferably comprise 1 to 18 carbon atoms, 1 to 15, carbon atoms, 1 to 12 carbon atoms, 1 to 8 carbon atoms, or 1 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure 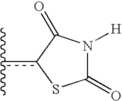
regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Inorganic radicals,” as the term is defined and used herein contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

“Organic radicals” as the term is defined and used herein contain one or more carbon atoms. An organic radical can have, for example, 1-26 carbon atoms, 1-18 carbon atoms, 1-12 carbon atoms, 1-8 carbon atoms, or 1-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds of the Invention

Some disclosed embodiments of the invention relate to a genus of compounds of Formula (I).

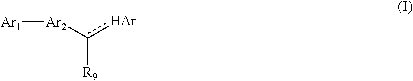

The compounds of Formula (I) comprise Ar₁ radicals that can be substituted dihydronapthalene or isochroman radicals, as are further disclosed and described immediately below.

In some embodiments of the the compounds of Formula (I), Ar₁ is a substituted isochroman radical having one of Formulas (Ar₁ₑ) or (Ar_{1f}) shown below:

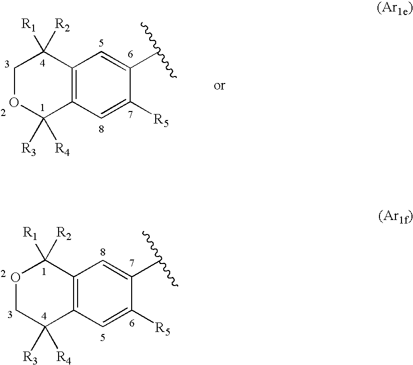

The (Ar₁ₑ) and (Ar_{1f}) radicals have five substituent radicals, R₁, R₂, R₃, R₄, and R₅, that can, in some embodiments, comprise any organic or inorganic substituent radical, as those terms are defined herein. In some of these embodiments, the (Ar₁ₑ) or (Ar_{1f}) radicals, together their substituent radicals R, R₂, R₃, R₄, and R₅ comprise from 9 to 25 carbon atoms, or from 10 to 20 carbon atoms, or from 12 to 18 carbon atoms, or from 14 to 16 carbon atoms.

The substituted isochroman radicals having Formulas (Ar₁ₑ) and (Ar_{1f}) comprise R₁, R₂, R₃, and R₄ substitutent radicals that are independently selected substitutent radicals at the “1” or “4” positions on the saturated pyran ring. In some embodiments, R₁, R₂, R₃, and R₄ are independently selected from hydrogen, halogen, amino, and/or substituents comprising 1 to 4 carbon atoms selected from alkyl, haloalkyl, cyano, mono-substituted amino, di-substituted amino, alkoxy, haloalkoxy, carboalkoxy, acyl, alkylcarboxamido, dialkylcarboxamido, alkylamido, and acyloxy, as those terms are defined elsewhere herein. In some embodiments, R₁, R₂, R₃ and R₄ are independently selected from alkyl groups comprising 1 to 4 carbon atoms. In some embodiments, all of R₁, R₂, R₃, and R₄ are methyl groups.

R₅ is a substituent radical for the benzene ring of the (Ar₁ₑ) and (Ar_{1f}) radicals, which is selected from the same radicals as were disclosed above for the Ar₁ₐ radicals. In some embodiments, R₅ is selected from hydrogen, a halogen, amino, sulfhydril, or a radical comprising 1 to 4 carbon atoms selected from alkyl, mono-substituted amino, di-substituted amino, alkoxy, haloalkoxy, thioalkyl, or thioacyl, as those terms are defined elsewhere herein. In some embodiments, R₅ is selected from methyl, a halogen (fluoro, chloro, bromo, or iodo), methoxy, amino, methylamino, or dimethylamino.

In yet further embodiments of the the compounds of Formula (I), Ar₁ is a substituted dihydronaphthalene radical having Formulas (Ar_{1g}) or (Ar₁ₕ), as shown below:

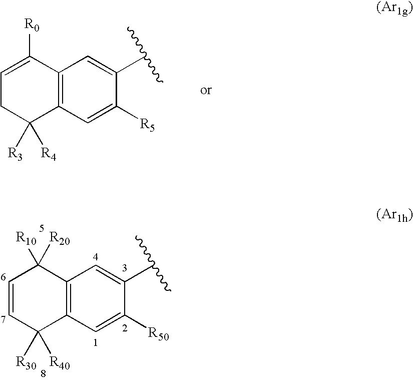

Both (Ar_{1g}) and (Ar₁ₕ) are dihydronaphthalene radicals in the conceptual and/or nomenclatural senses that two hydrogen atoms or other substituents have been “added” to one of the carbon-carbon double bonds of what would have otherwise been a napthlene radical, leaving one aromatic benzene ring fused to a cyclohexenyl ring. If the “added” substituents are at the “7” and “8” positions, radicals of Formula (Ar_{1g}) result. Alternatively, if the conceptually “added” substituents are placed on the “5” and “8” positions, radicals of Formula (Ar_{1f}) result.

The Ar_{1g} and Ar₁ₕ radicals have four or five substituent radicals, Rₒ, R₁₀, R₂₀, R₃₀, R₄₀, and/or R₅₀, that can, in some embodiments, comprise any organic or inorganic substituent radical, as those terms are defined herein. In some embodiments, the Ar_{1g} or Ar₁ₕ radicals have, together with their substituent radicals Rₒ, R₁₀, R₂₀, R₃₀, R₄₀, and R₅₀ comprise from 9 to 25 carbon atoms, or from 10 to 20 carbon atoms, or from 12 to 18 carbon atoms, or from 14 to 16 carbon atoms.

In some embodiments, the R₁₀, R₂₀, R₃₀, R₄₀, and R₅₀ radicals are independently selected from hydrogen, halogen, amino, and/or substituents comprising 1 to 4 carbon atoms selected from alkyl, haloalkyl, cyano, mono-substituted amino, di-substituted amino, alkoxy, haloalkoxy, carboalkoxy, acyl, alkylcarboxamido, dialkylcarboxamido, alkylamido, and acyloxy, as those terms are defined elsewhere herein. In some embodiments, the R₁, R₂, R₃, R₄ and R₅ radicals are all independently selected alkyl radicals comprising 1 to 4 carbon atoms, or are all methyl radicals.

The Rₒ substitutent group of the Ar₁g and Ar₁ₕ radicals are however unique. R₀ can be selected from hydrogen, a halogen, an aryl or heteroaryl comprising 1 to 8 carbon atoms, and radicals comprising 1 to 4 carbon atoms selected from alkyl, haloalkyl, di-substituted amino, alkoxy, haloalkoxy, or acyloxy. When Rₒ is an aryl or heteroaryl radical, there may be one or two additional organic or inorganic substituent radicals on the aryl or heteroaryl ring. Preferred aryl or heteroaryl Rₒ radicals can be phenyl, pyridyl, furanyl, thiofuranyl, or pyrrolyl radicals, with or without the additional substituents. In some embodiments, Rₒ is an alkyl radical comprising 1 to 4 carbon atoms, such as methyl, ethyl, n-propyl, i-propyl, n-butyl, i-butyl, t-butyl, and the like.

The compounds of Formula (I) also comprise an AR₂ aryl or heteroaryl radical, as those terms are defined elsewhere herein. AR₂ is bonded to both Ar₁ and to the carbon atom bearing R₉ that bridges to the HAr radical, and the bonds to the Ar₁ and carbon atoms can be at any chemically stable position on the Ar₂ ring, and in any chemically stable geometry relative to each other. Suitable AR₂ radicals include but are not limited to the monocyclic aromatic or heteroaromatic benzene, pyridine, pyrimidine, or pyrazine radicals having optional additional substitutents, as shown below. 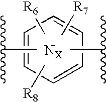
wherein X is an integer selected from 0, 1, or 2. If x is 0, a substituted phenyl radical results. If x is 1, a substituted pyridine radical results. If x is 2, a substituted pyrimidine, or pyrazine radical results. See the drawings immediately below for three examples.

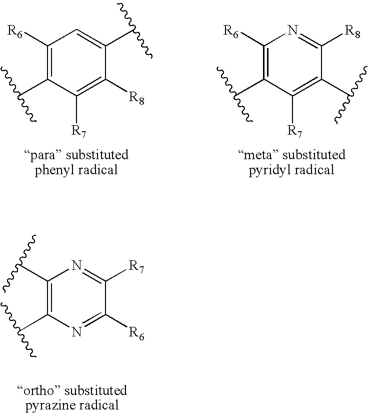

The R₆, R₇ and R₈ substituent radicals for AR₂ are independently selected from inorganic substituent radicals that include but are not limited to hydrogen, halogen, amino, nitro, and/or organic substituents comprising 1 to 4 carbon atoms which include but are not limited to alkyl, haloalkyl, cyano, mono-substituted amino, di-substituted amino, alkoxy, haloalkoxy, carboalkoxy, alkylcarboxamido, dialkylcarboxamido, alkylamido, acyloxy, —SH, thioalkyl, or thioacyl radicals, as those terms are defined elsewhere herein.

In many embodiments, the benzene, pyridine, pyrimidine, or pyrazine AR₂ radicals have a “meta” geometric relationship between the Ar₁ and bridging carbon atom substituents. The meta-substituted benzene, pyridine, pyrimidine, or pyrazine radicals have the generic structure shown below

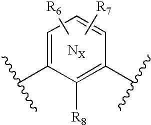

Examples of such meta-substuted AR₂ radicals include

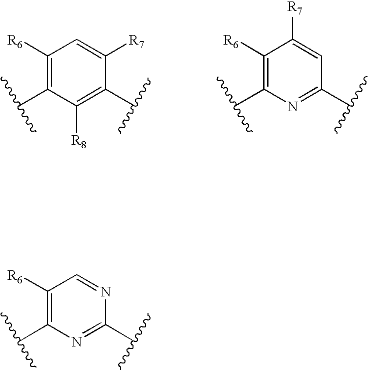

Although not wishing to be bound by theory, the AR₂ radical together with the substituent radicals R₆, R₇, and R₈ are selected so that the AR₂ radical has a geometry, size, and polarity that is suitable to induce the compounds of the invention to interact with and substantially fill, yet fit within the binding regions of the target biological molecules, so as to contribute to the effective binding of the compounds to the binding sites in the biological target molecules. Therefore, in some embodiments, the the AR₂ radical together with the substituent radicals R₆, R₇, and R₈ comprise from 4 to 18 carbon atoms, or from 5 to 16 carbon atoms, or from 6 to 14 carbon atoms, or from 7 to 12 carbon atoms.

As has been previously described, AR₂ has a “meta”substitution pattern with respect to Ar₁ and the carbon atom bearing R₉. It has been found that such a meta substitution pattern can result in unexpectedly superior biological activity for modulation of lipid and/or carbohydrate metabolism, and/or for the treatment of diseases of uncontrolled cellular proliferation, as compared to “ortho” or “para” AR₂ radicals.

Moreover, it has additionally been found that if a non-hydrogen R₆ substituent of an appropriate size and/or chemical character is present on the AR₂ radical, at the geometrical position illustrated in the drawings of the (Ar₂ₐ), (AR_{2b}), (AR_{2c}), and (AR_{2d}) radicals shown below, unexpectedly superior biological activities, such as the ability to simultaneously and beneficially modulate both carbohydrate and lipid metabolism, or inhibit the growth or progression of cancer cells, can result.

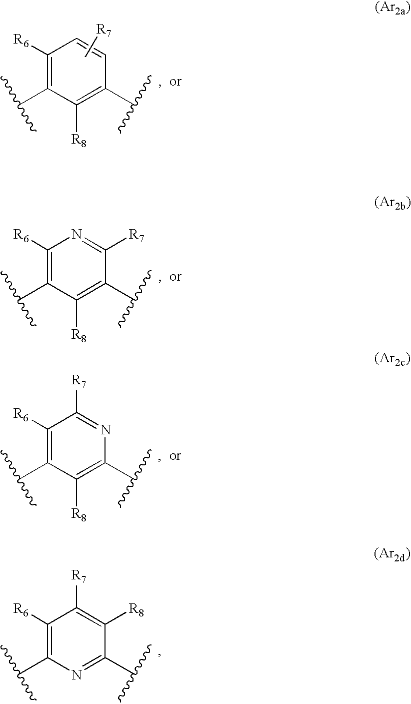

In the (AR₂ₐ), (AR_{2b}), (AR_{2c}), and (AR_{2d}) radicals, R₆ is selected from halogen, amino, or an organic substituent comprising 1 to 4 carbon atoms selected from alkyl, haloalkyl, cyano, mono-substituted amino, di-substituted amino, alkoxy, or haloalkoxy; and R₇ and R₈ are independently selected from hydrogen, halogen, amino, and substituents comprising 1 to 4 carbon atoms selected from alkyl, haloalkyl, cyano, mono-substituted amino, di-substituted amino, alkoxy, or haloalkoxy.

In some embodiments of the (AR₂ₐ), (AR_{2b}), (AR_{2c}), and (AR_{2d}) radicals, R₆ is a halogen, methyl, ethyl, n-propyl, i-propyl, trifluoromethyl, cyano, mono-methyl amino, di-methyl amino, methoxy, or trifluoromethoxy radical.

In some embodiments of the (AR₂ₐ), (AR_{2b}), (AR_{2c}), and (AR_{2d}) radicals, R₇ and R₈ are independently selected from hydrogen, fluorine, chlorine, bromine, or iodine.

Certain embodiments of the (AR₂ₐ) radicals that can be especially suitable include

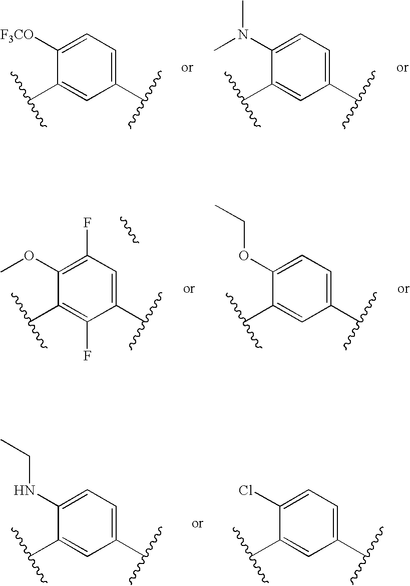

In further embodiments of the compounds of Formula (I), the AR₂ radicals can be a five membered heteroaryl radical, which can include but are not limited to substituted or unsubstituted thiofuran, furan, pyrrole, or pyrazole radicals that include the formulas:

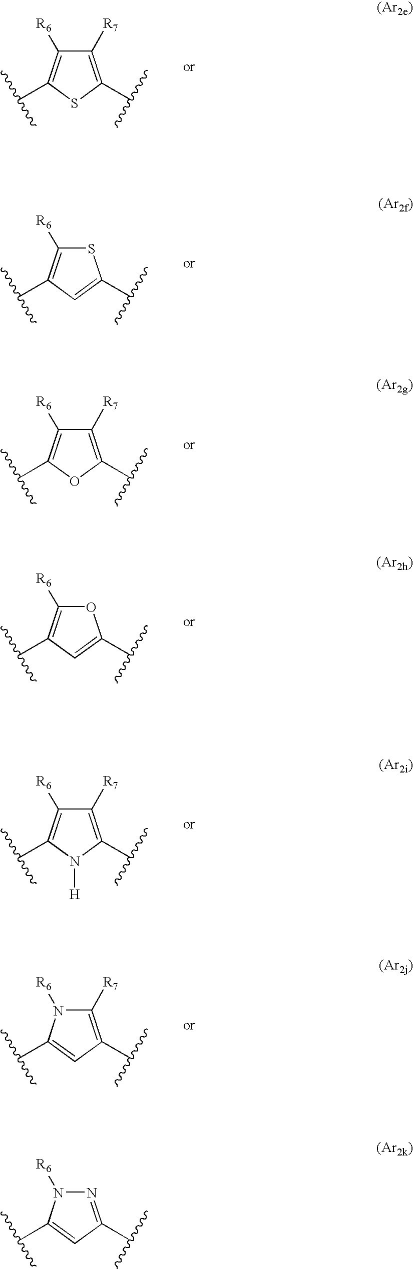

In the (AR₂ₑ)—(AR₂ₖ) ring radicals, R₆ and R₇ can have any of the same meanings as were listed above for R₆ and R₇ with respect to the (AR₂ₐ)—(AR_{2d}) radicals. Similar to the (AR₂ₐ)—(AR_{2d}) radicals, the presence of a non-hydrogen R₆ radical at the indicated positions can provide unexpectedly superior biological activity.

As disclosed above, in the compounds of Formula (I), the AR₂ radical is bonded to a carbon atom which bridges to the HAr heterocyclic radicals. As described below, the bridging carbon atom can be either a methine or a methylene carbon atom, depending on whether or not there is an optional carbon-carbon double bond to the HAr heterocycle. Accordingly, as shown in Formula (I), the carbon-carbon double bond, as illustrated by 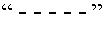
can be either present or absent.

In any case, the bridging methylene and methine carbon atoms can have one or two R₉ substituents, which can be independently selected from hydrogen, hydroxy, or an alkyl radical comprising 1 to 4 carbon atoms. In many embodiments, a carbon-carbon double bond is present, and the carbon atom is a methylene carbon atom bearing a single R₉ substitutent, which is hydrogen.

The compound of Formula (I) also comprise a five membered heterocyclic HAr radical selected from a 2,4-thiazolidinedione, 2-thioxo-thiazolidine-4-one, 2,4-imidazolidinedione or 2-thioxo-imidazolidine-4-one residue, as shown in the drawings below:

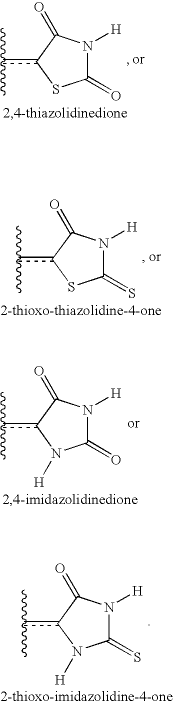

In many embodiments, 2,4-thiazolidinedione or 2-thioxo-thiazolidine-4-one radicals are selected for use as the HAr radical. In many embodiments, 2,4-thiazolidinedione is uniquely selected for use as the HAr radical.

In some embodiments 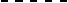
represents a bond present and the compound is a benzylidene compound having Formula (Ia):

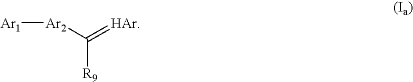

When 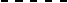
is present both E and Z configurations of the carbon-carbon bond between the benzylidene carbon atom and the HAr heterocycle are within the scope of the invention. Either isomer can predominate or be present in pure form, or in a mixture, which may or may not have equal proportions of the E and Z isomers. For example, 2,4-thiazolidinedione and 2-thioxo-4-thiazolidinedione of Formula (200) can have the following structures respectively:

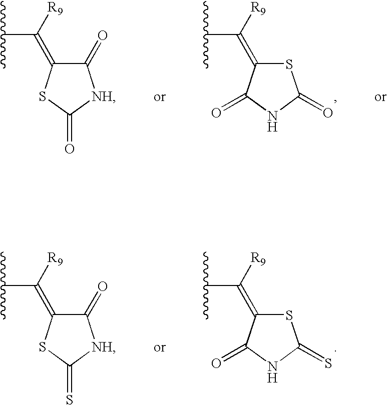

When only one of the two isomers is shown in this specification or in the claims, it should be presumed that both pure isomers, as well as mixtures thereof are intended unless the context makes it plain that only a single isomer is intended.

In some embodiments 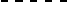
represents a bond absent and the compound is a benzyl compound with a single carbon-carbon bond between a benzylic carbon and the HAr ring, the compounds having the Formula (Ib): 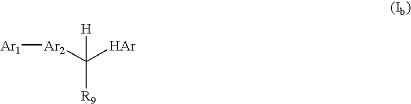
wherein HAr and the benzylic carbon atom bonded thereto would have the formulas

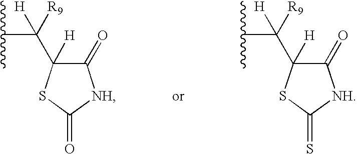

Those of ordinary skill in the art will recognize that for the compounds illustrated in the drawings immediately above, the benzylic carbon and the carbon of the HAR ring bond to the benzylic carbon can potentially be optically present in the form of only one of two possible absolute configurations (R or S) so as to be optically active, or in the form mixtures of the two optical isomers in any proportion, including racemic mixtures. Compounds having either pure optical isomer at either position, or racemic mixtures are within the scope of the invention, as are all the erythro and threo diastereomers formed if both the positions are in optically active form.

The effectiveness of a particular compound of Formula (I) as a therapeutic agent can depend on its ability to bind to the relevant biological target's binding sites, but can also be dependent to a significant degree on the overall physiochemical properties of that individual compound. This occurs because pharmaceutical properties such as degree and rate of compound absorption and/or bioavailability depend on molecular weight, lipophilicity, aqueous solubility, and various other physiochemical properties. Theses properties can substantially vary upon small structural changes, thus compounds with similar structures can have dissimilar pharmaceutical properties. In the present invention, physiochemical properties such as cLogP, polar surface area and cLogD, were in many cases predicted and/or calculated for a particular compound before its synthesis, or alternatively measured after the synthesis of the individual compound, such as for example melting point, LogP (i.e log of the octanol/water partition coefficient), solubility, etc, so as to enable the selection compounds that have a structure that is likely to both bind the biological target's binding sites, and to have a desirable combination of physiochemical properties. Thus, the compounds of Formula (I), because of the introduction of certain polar heteroatoms or olefinic groups, or heteroatomic substituents on their Ar₁ and AR₂ radicals, can exhibit unexpectedly superior physiochemical properties as compared to many prior art compounds, which result in unexpectedly improved formulation properties and in vivo activity, as compared to prior art compounds that may act on similar biological targets.

As already noted above, the HAr ring radical of the compounds of Formula (I) is selected from one of four heterocycles, shown in the drawing below:

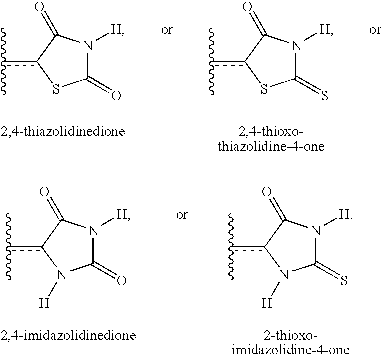

All four of the HAr heterocycles shown above comprise at least one ring nitrogen atom bonded to a hydrogen atom. The nitrogen-bound hydrogen atoms of the HAr heterocycles are known to be sufficiently acidic so as to react with common laboratory bases such as organic amine compounds, hydroxide salts, and the like. The acidity of the four HAr heterocycles provides a ready method for preparing salts of the compounds of the invention, by reaction with an appropriate base, so as to generate an anion derived from the compound of Formula (I) and a cation derived from the base employed to neutralize the compound of Formula (I). The salts formed by such reactions can have, for example, the formula

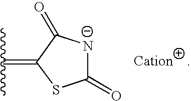

A wide variety of bases could be employed to produce such salts, including monovalent alkali metal hydroxides, divalent alkaline earth metal hydroxides, or bases comprising trivalent metal salts such as aluminum. Alternatively, organic bases such as primary, secondary, or tertiary amines can react with the acidic hydrogens of the compounds of the invention to form ammonium salts. The base and/or its associated cation are chosen so as to provide desirable solubility, toxicity, and/or bioavailability characteristics in the salt after formation of the desired salts. The identity of the base and/or the resulting cation will of course vary somewhat with the identity of the compound of the invention, and the nature of the pharmaceutical composition to be employed and its physical form as a solid or liquid, and the nature of any solvents and/or carriers employed.

Nevertheless, the United States Food and Drug Administration has published a list of pharmaceutically acceptable cations for use in pharmaceutically acceptable salts that includes aluminum, calcium, lithium, magnesium, potassium, sodium, and zinc cations, ammonium cations formed by the reactions of acidic compounds with benzathine, chloroprocaine, choline, diethanolamine, ethylenediamine, meglumine, procaine, t-butylamine, and tris(hydroxymethyl)aminomethane (“Tris”). Such “pharmaceutically acceptable” salts are often employed in the invention simply because of the increased likelihood of pharmaceutically acceptable properties and/or decreased level of FDA regulatory scrutiny to be expected. Example 12 provides an example of the synthesis of a particularly useful “Tris” salt of one of the compounds of the invention.

Also, one or more compounds disclosed herein can include zwitterionic salts formed by reaction of a nitrogen contained internally within the compound, such as an amine, aniline, substituted aniline, pyridyl, and like residues with the acidic hydrogen of the HAr group. Alternatively, a basic nitrogen atom contained internally within the compound can be reacted with an external acid, such as HCl, sulfuric acid, a carboxylic acid or the like, to form a cationic form of the compounds of Formula (I).

Compounds disclosed herein can exist in various tautomeric forms. For example, 2,4-thiazolidinedione-containing compounds disclosed herein can exist in the form of tautomers (I_{c}), (I_{d}) and (Iₑ).

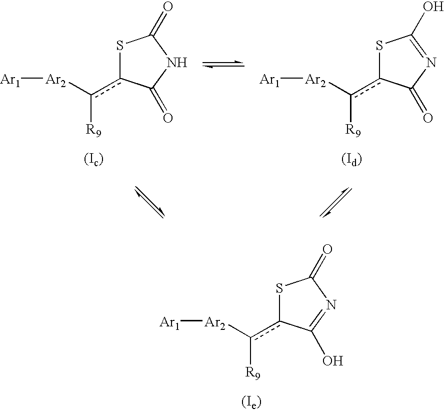

It is understood by those of skill in the art that tautomers can also exist with compounds of the invention that contain the heterocycle 2-thioxo-thiazolidine-4-one, 2,4-imidazolidinedione or 2-thioxo-imidazolidine-4-one. For convenience, all of the tautomers can be presented herein by a single formula, but it is understood that all tautomers are within the scope of the invention.

Selected embodiments of the compounds of Formula (I) can be described more narrowly than the broadest embodiments described above. Two examples of such narrower descriptions are set forth below, but the meanings of the various relevant terms and symbols are intended the same as those same terms and symbols in the description above.

In one embodiment of the compounds of Formula (I), the invention relates to isochroman compounds having the structure 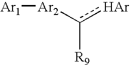
wherein
a) Ar₁ has the structure 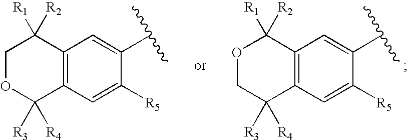
wherein R₁, R₂, R₃, and R₄ are independently selected from hydrogen, halogen, amino, and/or substituents comprising 1 to 4 carbon atoms selected from alkyl, haloalkyl, cyano, mono-substituted amino, di-substituted amino, alkoxy, haloalkoxy, carboalkoxy, acyl, alkylcarboxamido, dialkylcarboxamido, alkylamido, acyloxy; and R₅ is selected from hydrogen, a halogen, amino, sulfhydril, or a radical comprising 1 to 4 carbon atoms selected from alkyl, mono-substituted amino, di-substituted amino, alkoxy, haloalkoxy, thioalkyl, or thioacyl;
b) AR₂ has the structure 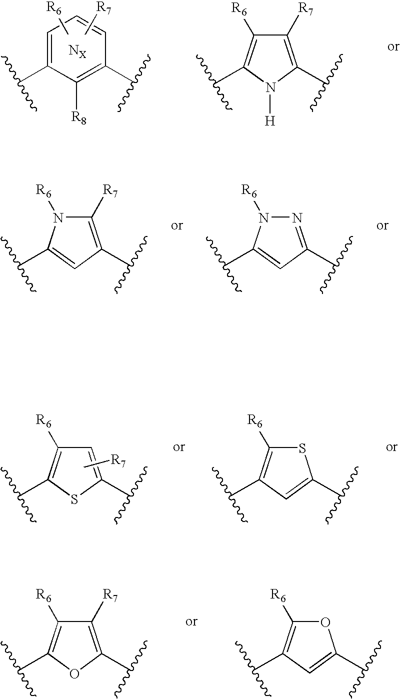
wherein X is an integer selected from 0, 1, or 2, and R₆, R₇ and R₈ are independently selected from hydrogen, halogen, amino, nitro, and/or substituents comprising 1 to 4 carbon atoms selected from alkyl, haloalkyl, cyano, mono-substituted amino, di-substituted amino, alkoxy, haloalkoxy, carboalkoxy, alkylcarboxamido, dialkylcarboxamido, alkylamido, acyloxy, sulfhydril, thioalkyl, or thioacyl;
c) R₉ is hydrogen, hydroxy, or an alkyl radical comprising 1 to 4 carbon atoms;
d) 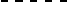
is either present or absent;
e) HAr is a heterocycle having the structure 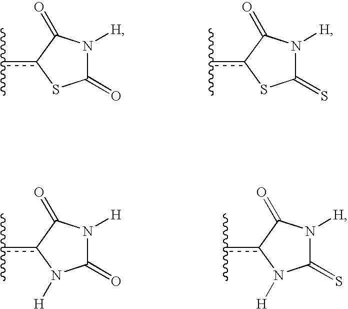
or a pharmaceutically acceptable salt thereof.

In yet another embodiment of the compounds of Formula (I), the invention relates to dihydronaphthalene compounds having the structure 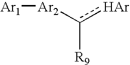
wherein
a) Ar₁ has the structure 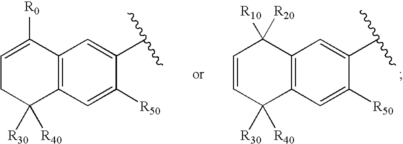
wherein R₀ is selected from hydrogen, a halogen, an aryl or heteroaryl comprising 1 to 8 carbon atoms, and radicals comprising 1 to 4 carbon atoms selected from alkyl, haloalkyl, di-substituted amino, alkoxy, haloalkoxy, or acyloxy; and R₁₀, R₂₀, R₃₀, and R₄₀. are independently selected from substituents comprising 1 to 4 carbon atoms selected from alkyl, haloalkyl, cyano, amino, mono-substituted amino, di-substituted amino, alkoxy, haloalkoxy, carboalkoxy, alkylcarboxamido, dialkylcarboxamido, alkylamido, or acyloxy, and R₅₀ is selected from hydrogen, a halogen, amino, sulfhydril, or a radical comprising 1 to 4 carbon atoms selected from alkyl, mono-substituted amino, di-substituted amino, alkoxy, haloalkoxy, thioalkyl, or thioacyl;
b) AR₂ has the structure 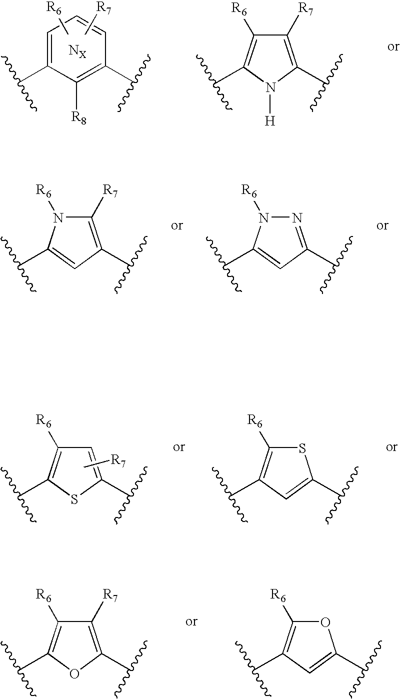
wherein X is an integer selected from 0, 1, or 2, and R₆, R₇ and R₈ are independently selected from hydrogen, halogen, amino, nitro, and/or substituents comprising 1 to 4 carbon atoms selected from alkyl, haloalkyl, cyano, mono-substituted amino, di-substituted amino, alkoxy, haloalkoxy, carboalkoxy, alkylcarboxamido, dialkylcarboxamido, alkylamido, acyloxy, sulfhydril, thioalkyl, or thioacyl;
c) R₉ is hydrogen, hydroxy, or an alkyl radical comprising 1 to 4 carbon atoms;
d) 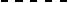
is either present or absent;
e) HAr is a heterocycle having the structure 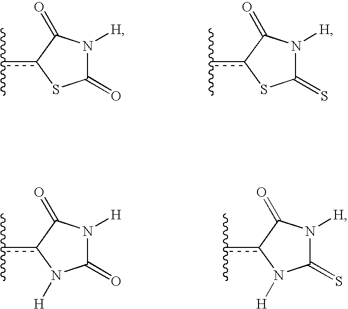
or a pharmaceutically acceptable salt thereof.

In yet another embodiment of the compounds of Formula (I), the invention relates to dihydronaphthalene compounds having the structure 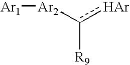
wherein
a) Ar₁ is has the structure 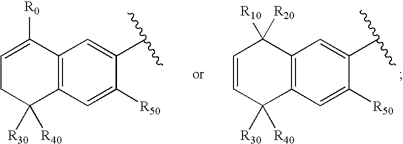
wherein R₀ is selected from an aryl or heteroaryl comprising 1 to 8 carbon atoms, and alkyl radicals comprising 1 to 4 carbon atoms; and R₁₀, R₂₀, R₃₀, and R₄₀. are independently selected from alkyl radicals comprising 1 to 4 carbon atoms; and R₅₀ is selected from hydrogen, a halogen, amino, sulfhydril, or a radical comprising 1 to 4 carbon atoms selected from alkyl, mono-substituted amino, di-substituted amino, alkoxy, haloalkoxy, thioalkyl, or thioacyl;
b) AR₂ has the structure 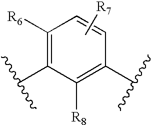
wherein R₆ is a halogen, methyl, ethyl, n-propyl, i-propyl, trifluoromethyl, cyano, mono-methyl amino, di-methyl amino, methoxy, or trifluoromethoxy radical, and R₇ and R₈ are independently selected from hydrogen, fluorine, chlorine, bromine, or iodine;
c) R₉ is hydrogen;
d) 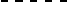
is either present or absent;
e) HAr is a heterocycle having the structure 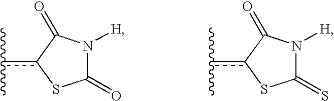
or a pharmaceutically acceptable salt thereof.

In yet another embodiment of the compounds of Formula (I), the invention relates to isochroman compounds having the structure 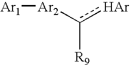
wherein
a) Ar₁ is has the structure 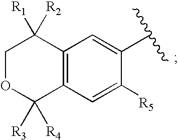
wherein R₁, R₂, R₃ and R₄ are independently selected alkyl groups comprising 1 to 4 carbon atoms; and R₅ is selected from methyl, a halogen (fluoro, chloro, bromo, or iodo), methoxy, amino, methylamino, or dimethylamino;
c) AR₂ has the structure 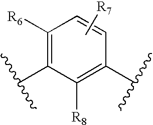
wherein R₆ is a halogen, methyl, ethyl, n-propyl, i-propyl, trifluoromethyl, cyano, mono-methyl amino, di-methyl amino, methoxy, or trifluoromethoxy radical, and R₇ and R₈ are independently selected from hydrogen, fluorine, chlorine, bromine, or iodine;
d) R₉ is hydrogen;
e) 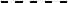
is present;
f) HAr is a heterocycle having the structure 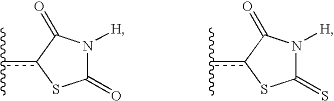
or a pharmaceutically acceptable salt thereof.

The present invention also relates to, but is not limited to, the specific species compounds set forth in the Examples, or a pharmaceutically acceptable salt thereof.

This invention also encompasses pharmaceutical compositions containing prodrugs of the compounds of the invention as disclosed herein. The term “prodrug” means a drug precursor which, following administration, releases the drug (e.g., a compound of the present invention) in vivo via some chemical or physiological process. For example, a prodrug on being brought to the physiological pH or through enzyme action is converted to the desired drug form. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A. C. S. Symposium Series, and in Bioreversible Camers in Drug Design, ed. Edward B. Roche, American Pharmaceutical Association and Pergamon Press, 1987, the text of both of which treatises is hereby incorporated herein by reference, for their teachings regarding the structures, uses, properties, and preparations of prodrugs.

For example, if a compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as .beta.-dimethylaminoethyl), carbamoyl-(C₁-C₂)alkyl, N,N-di(C₁₋₂)alkylcarbamoyl-(C₁-C₂)alkyl and piperidino-, pyrrolidino- or morpholino(C₂-C₃)alkyl.

Similarly, if a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, .alpha.-amino(C₁-C₄)alkanoyl, arylacyl and .alpha.-aminoacyl, or .alpha.-aminoacyl-.alpha.-aminoacyl, where each .alpha.-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention comprises an amine functional group, a prodrug can be formed by the replacement of a hydrogen atom in the amine group with a group such as R-carbonyl, RO-carbonyl, NRR′-carbonyl where R and R′ are each independently ((C₁-C₁₀)alkyl, (C₃-C₇)cycloalkyl, benzyl, or R-carbonyl is a natural .alpha.-aminoacyl or natural .alpha.-aminoacyl-natural .alpha.-aminoacyl, —C(OH)C(O)OY wherein (Y is H, (C₁-C₆)alkyl or benzyl), —C(OY₀)Y₁ wherein Y₀ is (C₁-C₄)alkyl and Y₁ is ((C₁-C₆)alkyl, carboxy(C₁-C₆)alkyl, amino(C₁-C₄)alkyl or mono-N— or di-N,N-(C₁-C₆)alkylaminoalkyl, —C(Y₂)Y₃ wherein Y₂ is H or methyl and Y₃ is mono-N— or di-N,N-(C₁-C₆)alkylamino, morpholino, piperidin-1-yl or pyrrolidin-1-yl.

Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula 1. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, omithine and methionine sulfone. Prodrugs also include compounds wherein carbonates, carbamates, amides and alkyl esters which are covalently bonded to the compounds of formula I or II. The prodrugs themselves may be in the form of a pharmaceutically acceptable salt.

Making Compounds of the Invention

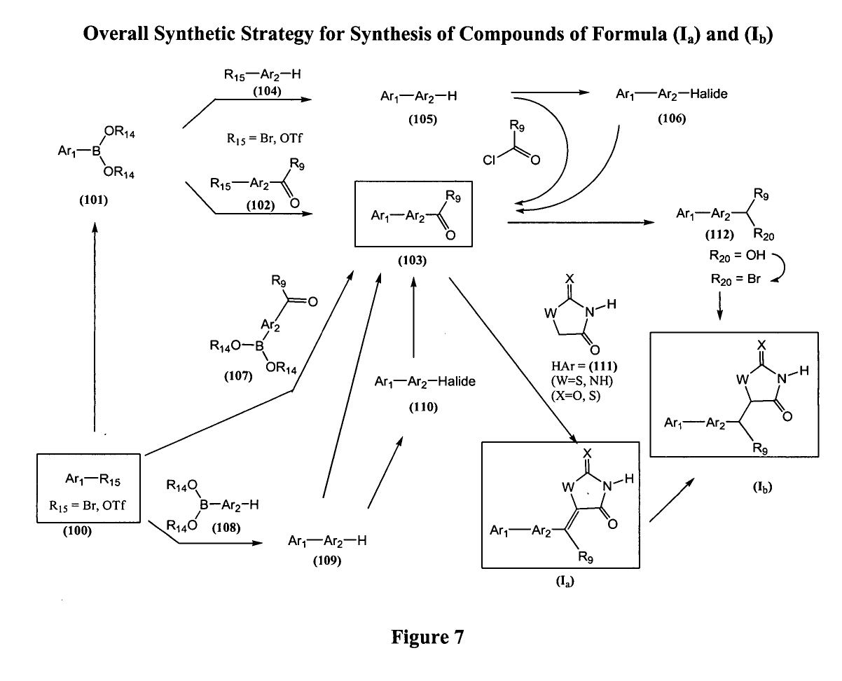

Various synthetic methods can be employed in the making of the compounds of Formula (I) disclosed herein. A representative set of synthetic pathways that can be employed to couple precursors of the AR₁, AR₂ and HAr radicals together to form compounds of Formula (I) is shown in FIG. 7.

Typical precursors of Ar₁ are aryl halides (especially bromides) or aryl triflates of Formula (100). Suitable synthesis of compounds of Formula (100) will be further detailed hereinbelow. The synthetic precursors of AR₂ are also typically aryl or heteroaryl halides or triflates of Formula (102), which also comprise a carbonyl functional group. A large number of suitable aromatic precursor compounds of Formula (102) are readily available commercially available from suppliers such as Aldrich Chemical Company of Milwaukee Wis., or can be prepared by the extensive and well known and traditional methods of organic chemistry as applied to aromatic compounds that are well known to those of ordinary skill in the art. Such knowledge of those of ordinary skill in the art of the synthesis of organic compounds is summarized in many well known texts and treatises, which include, for example, March, J., Advanced Organic Chemistry, 4^{th} Edition, Weiley-Interscience (1992); or Larock, R. C., Comprehensive Organic Transformations, A Guide to Functional Group Preparations, VCH Publishers, Inc. (1989), both of which are hereby incorporated herein by reference in their entirity.

In many embodiments of the invention, the coupling of a desired Ar₁ radical (101) with a desired AR₂ radical (102) to produce the desired biaryl carbonyl compound (103) shown in FIG. 7 is conducted using a palladium catalyzed “Suzuki” coupling of an aryl boronic acid or ester with an aryl halide (such as, iodo, bromo, or chloro), triflate or diazonium tetrafluoroborate; as described respectively in Suzuki, Pure & Applied Chem., 66:213-222 (1994), Miyaura and Suzuki, Chem. Rev. 95:2457-2483 (1995), Watanabe, Miyaura and Suzuki, Synlett. 207-210 (1992), Littke and Fu, Angew. Chem. Int. Ed., 37:3387-3388 (1998), Indolese, Tetrahedron Letters, 38:3513-3516 (1997), Firooznia, et. al., Tetrahedron Letters 40:213-216 (1999), and Darses, et al., Bull. Soc. Chim. Fr. 133:1095-1102 (1996); all incorporated herein by reference. According to this coupling reaction, precursors such as (101) and (102) may be employed: 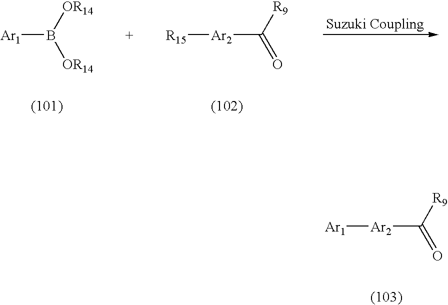
wherein R₁₄ is either alkyl or hydrogen, and R₁₅ is a halide (such as, iodo, bromo, or chloro), triflate or diazonium tetrafluoroborate. As is shown in FIG. 7, the aryl borate (101) can be prepared by lithiation of a precursor aryl halide (such as, iodo, bromo) (100), followed by treatment with a boric acid triester or can be prepared by palladium-catalysed cross coupling reaction of a precursor aryl halide (such as, iodo, bromo, or chloro) or triflate (100) with pinacol borane. Coupling reactions to produce biaryls such as (103) may be conducted using either aryl boric acids, or aryl boronic esters, including cyclic esters in which two of the R₁₄ groups together with the boron atom from a pinacol borate ester (formation of pinacol borane esters: Ishiyama, T., et al., J. Org. Chem. 1995, 60, 7508-7510, Ishiyama, T., et al., Tetrahedron Letters 1997, 38, 3447-3450; coupling pinacol borane esters: Firooznia, F. et al., Tetrahedron Letters 1999, 40, 213-216, Manickam, G. et al., Synthesis 2000, 442-446; wherein all four citations are hereby incorporated herein by reference in their entireties). In addition, R₁₅ may also be I, Cl or triflate (derived from a phenol).

Alternatively, a “reverse” Suzuki coupling strategy also shown in FIG. 7 can be employed, in which the roles of the borate and the halide/triflate functional groups are switched, as shown below, yet achieve the same final coupling product (103). 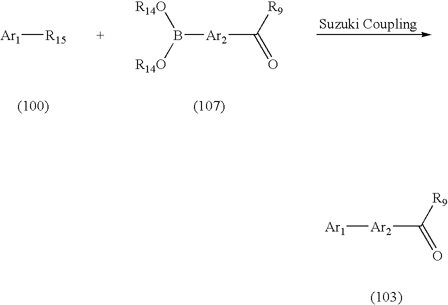
wherein R₁₄ and R₁₅ have the same meaning as described above. Depending on the ease of availability and/or structure and reactivity of the appropriate starting materials, the “normal” and “reverse” couplings can be similarly employed, or one or the other may be advantageous.

In an alternative method for preparing compounds of Formula (103) shown in FIG. 7, boronic acid (101) may be coupled with an aryl bromide that does not contain a carbonyl group (104), to give biaryl (105), which can be subsequently acetylated or formylated using techniques known in the art, such as the Friedel-Craft acylations, or the Vilsmeier or the Vilsmeier-Haack reaction, the Gatterman reaction, the Duff reaction, the Reimer-Tiemann reaction or a like reaction. Biaryl (105) can also be formulated or acylated, for example by the Friedel-Crafts acylation reaction, or the like, to produce compound (103) in a single step. Alternatively, in a two step method, biaryl (105) is first halogenated to give biaryl-halide (106), such as a bromination, followed by a halogen-metal exchange reaction using an alkyl lithium, followed by reaction of the aryl lithium intermediate with dimethylformamide, or an equivalent known in the art, to give compounds of Formula (103). As can be seen from FIG. 7 and compounds (108), (109), and (110), a similar reaction sequence may be employed with “reverse” Suzuki coupling reactions. In any event, one of ordinary skill in the art will understand that these various alternative methods, as well as other known methods of organic chemistry can be used to produce compounds of Formula (103) having a variety of desirable substitution patterns.

As shown in FIG. 7, in many embodiments of the methods for producing the compounds of Formula (I), the carbonyl group of biaryl (103) is condensed in a “Knoevenagle” reaction with a heterocyclic precursor of the HAr radical (111) possessing an active methylene moiety, such as 2,4-thiazolidinedione, 2-thioxo-4-thiazolidinedione, isoxazolidinedione, 2,4-imidazolidinedione or 2-thioxo-4-imidazolidinedione, to give a desired final product benzylidene compound having Formula (Iₐ).

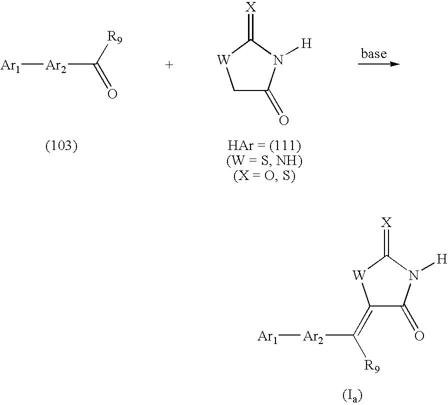

Condensation of the biaryl carbonyl derivatives (103) with a suitable active methylene compound, such as, 2,4-thiazolidinedione, can be accomplished by the use of methods known in the art. Similar reactions have been described by Tietze and Beifuss, Comprehensive Organic Synthesis (Pergamon Press), 2:341-394, (1991), incorporated herein by reference. Effective catalysts for the condensation can be selected from ammonia, primary, secondary and tertiary amines, either as the free base or the amine salt with an organic acid, such as acetic acid. Examples of catalysts include pyrrolidine, piperidine, pyridine, diethylamine and the acetate salts thereof. Inorganic catalysts can also be used for the condensation. Inorganic catalysts include, but are not limited to, titanium tetrachloride and a tertiary base, such as pyridine; and magnesium oxide or zinc oxide in an inert solvent system. This type of condensation can be strongly solvent-dependent and it is understood that routine experimentation may be necessary to identify the optimal solvent with a particular catalyst, preferable solvents include ethanol, tetrahydrofuran, dioxane or toluene; or mixtures thereof.

It is understood by those skilled in the art that intermediates having hydroxyl groups bonded thereto can be formed during condensation of an biaryl carbonyl containing derivative (103) and an active methylene compound, as shown below.

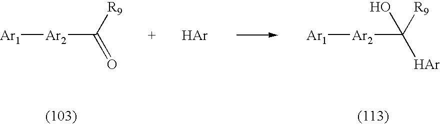

The hydroxyl group of intermediate (113) is often eliminated (as water) during the condensation reaction, to form the desired benzylidene compound (Iₐ). Nevertheless, the conditions of the reaction can be modified for the isolation or further use of the hydroxyl containing intermediates, and such embodiments are within the scope of the invention.

In an optional additional step, the carbon-carbon double bond of benzylidene compound of Formula (Iₐ) may be reduced/hydrogenated by any of a variety of known methods for reducing double bonds, to give a benzyl compound of Formula (I_{b}) having only a carbon-carbon single bond to the HAr heterocycle.

In yet another alternative method shown in FIG. 7, the carbonyl group of biaryl (103) is reduced, for example with sodium borohydride, to give benzyl alcohol (112, R₂₀═OH), then converted with HBr or some other method known in the art, such as PPh₃/CBr₄ to give the benzyl bromide (112, R₂₀═Br). Benzyl bromide (112) is reacted with an anion of one of the HAr precursors, such as 2,4-thiazolidinedione, to give the reduced final product benzyl compound having Formula (I_{b}).

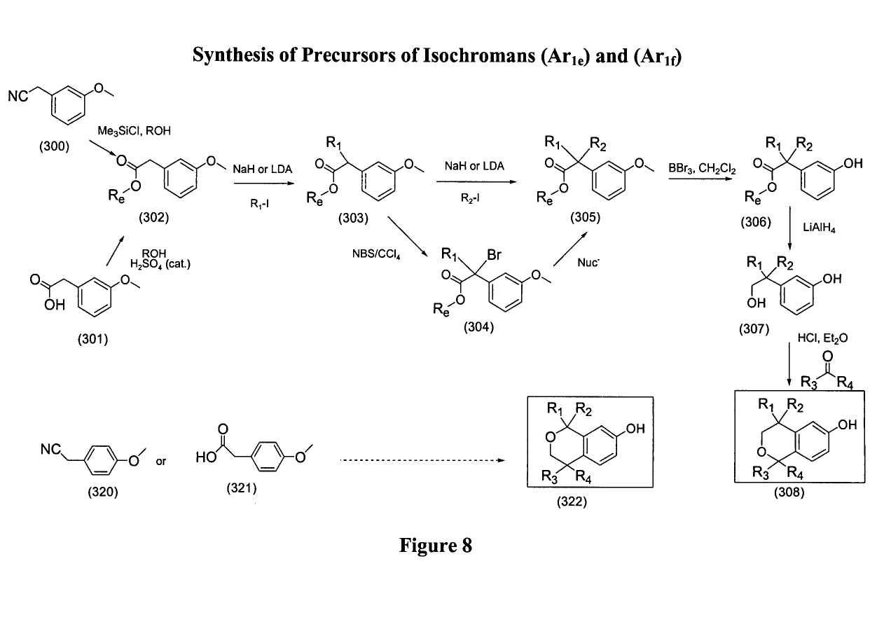

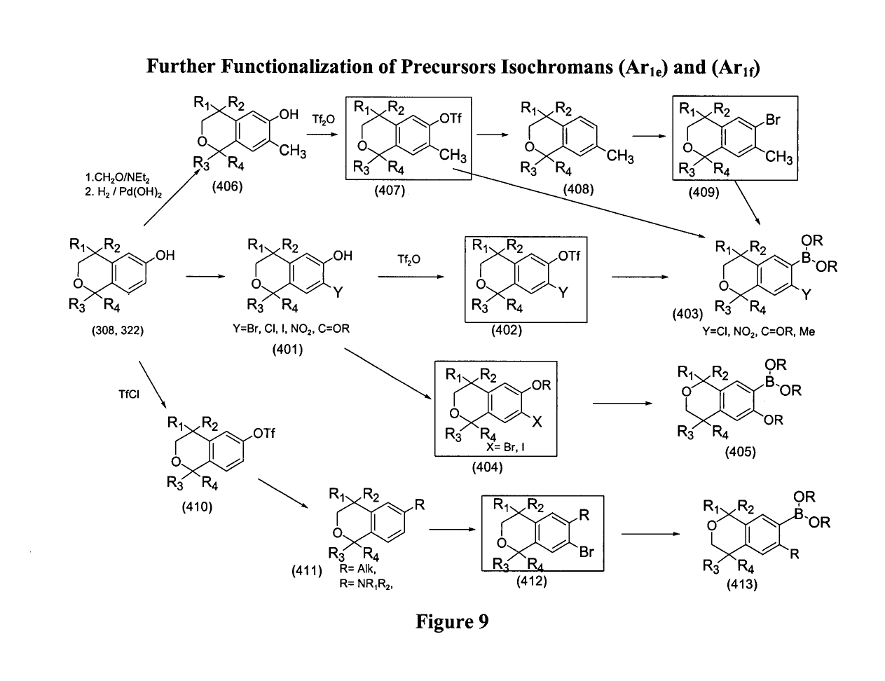

Various methods for synthesizing suitable synthetic precursors of the Ar₁ radicals will now be described and/or illustrated in FIGS. 8-10.

A representative set of synthetic pathways for synthesizing precursors of the isochroman radicals of Formula (Ar₁ₑ) and (Ar_{1f}) are shown in FIG. 8. Suitable starting materials are the methoxyphenyl-acetonitriles (300) and (320), or the methoxyphenyl acetic acids of structure (301) or (321), all of which are commercially available from suppliers such as Aldrich Chemical Company of Milwaukee Wis. The methoxyphenyl-acetonitrile (300) or the methoxyphenyl acetic acid (301) can be reacted with a suitable alcohol, such as MeOH, EtOH, etc, as shown in FIG. 8 to provide the the methoxyphenyl acetic acid ester (302), which has reactive hydrogens at the benzylic position that can be elaborated via a variety methods to introduce the R₁ and R₂ substituents of compound (305). For example, the two benzylic hydrogens of (302) can be readily removed with strong bases such as sodium hydride (NaH) or lithium di-isopropyl-amide (LDA) to give nucleophillic anions that can be reacted with alkylating agents such as alkyl halides or alkyl sulfates to introduce the R₁ group of compound (303), or if the reaction sequence is repeated with a second alkylating agent, the R₂ group of compound (305). Alternatively, the mono-alkylated compound (303) can be halogenated at the benzyllic carbon atom with free radical halogentation agents, such n-bromo-succinimide in carbon tetrachloride, to give halogenated compounds such as (304).

The bromide of compound (304) can be displaced by a variety of nucleophiles, such as alkoxides, amines, thiolates, carboxylates, and the like, to give compounds of Formula (305) having different R₁ and R₂ groups. The methoxy group of compound (305) can be removed and the carboxylic ester reduced with lithium aluminum hydride to produce the disubstituted 2-hydroxyethyl phenol (307), which can then be condensed with a ketone to close the ring, introduce the R₃ and R₄ substituents, and form the desired precursor isochrom-ol compound (308). The phenolic isochrom-ol compound (308) is a direct precursor of Ar₁, in that it can be readily tosylated to form a tosylate ester suitable for Suzuki coupling to AR₂ radicals, as will be discussed hereinbelow.

As is also illustrated at the bottom of FIG. 8, simply repeating the reaction sequences just described starting with the appropriate isomers of compounds (320) or (321), the phenolic isochromans (322) can be prepared, which are effective precursors of the (Ar_{1f}) isochroman radicals of the compounds of Formula (I).

Methods for the introduction of certain desirable R₅ substitutents onto the aromatic ring of precursors of the isochroman rings having structures (Ar₁ₑ) and (Ar_{1f}) are shown in FIG. 8. Isochromans (308) and (322), or tetrahydroisoquinolines (312) or (333) can be subjected to traditional aromatic electrophillic substitution reactions to introduce a variety of halogen, nitro, or acyl substitutents “Y,” to yield compounds of Formula (401), which can be triflated to form triflate esters such as (402), which can in many cases be directly “reverse” Suzuki coupled with a borate ester precursor of AR₂, or triflate ester (402) can be converted to a borate ester having Formula (403), which is suitable for “normal” Suzuki couplings.

Isochromans (308) and (322) can be reacted with formaldehyde and diethylamine, and then hydrogenated over palladium hydroxide, in analogy to the procedure disclosed in Organic Preparations and Procedures Int., 25 (2) 223-228 (1993), to yield methylated compounds of Formula (406). The phenolic hydroxyl of compound (406) can be triflated to directly form a suitable synthetic triflate ester precursor (407) of compounds of formulas (Ar_{1c}) or (Ar₁ₑ). The triflate esters (407) can in many cases be directly “reverse” Suzuki coupled with a borate ester precursor of AR₂. Alternatively, triflate ester (407) can be reduced by the procedure of Cacchi S. et al [Palladium-Catalyzed triethylammoniumformate reduction of aryl triflates. A selective method for the deoxygenation of phenols, Tetrahedron Letters, 27 (45), pp 5541-5544, (1986)], so as to provide compound (408), which can be brominated to produce compound (409). which can be converted to a borate ester having Formula (403) suitable for “normal” Suzuki couplings, or also utilized directly in a “reverse” Suzuki coupling.

In yet further embodiments of the methods of synthesizing the compounds of the invention, the phenolic hydroxyl group of fisochromans (308) and (322) can be tosylated and then displaced with a nucleophile such as an alkyl group (in analogy to the procedure described in Tetrahedron Letters 41 (2000) 6237-6240), or a dialkyl amine (in analogy to the procedure disclosed in J. Org. Chem. 1997, 62, 1268-1273) to provide compound (411), which can be brominated to provide compound (412), which can be converted to a borate ester having Formula (403) suitable for “normal” Suzuki couplings, or also utilized directly in a “reverse” Suzuki coupling.

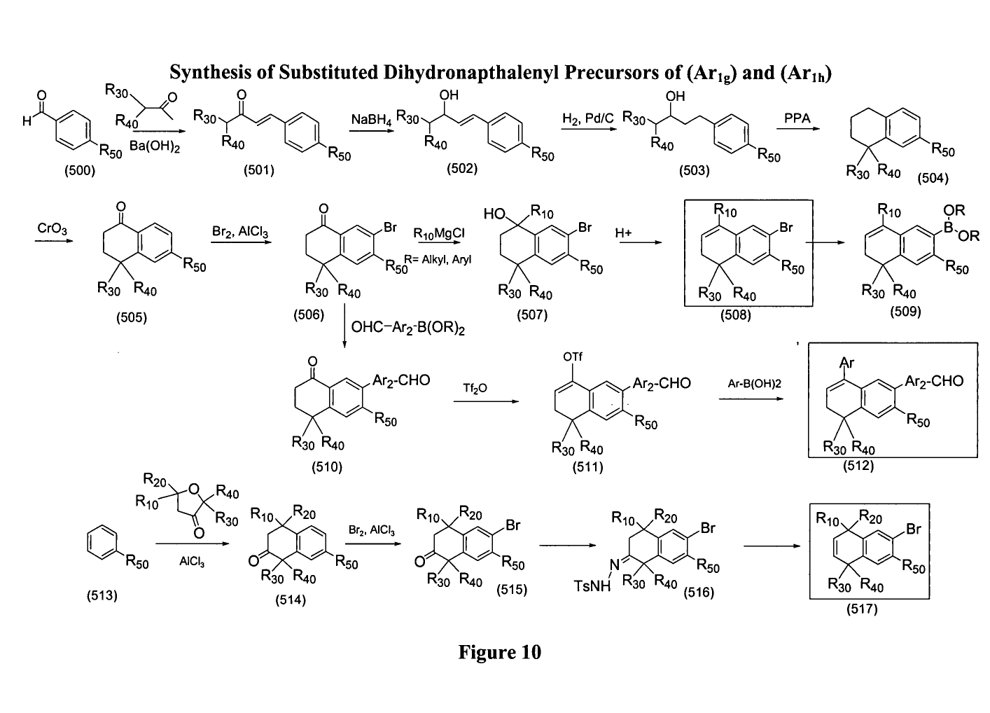

In yet other embodiments of the invention, a representative set of synthetic pathways for synthesizing precursors of the dihydronaphthalenyl radicals of Formula (Ar_{1g}) and (Ar₁ₕ), are shown in FIG. 10. Suitable starting materials are the substituted benzaldehydes (500), or the substituted benzene compounds of structure (513), many of which are commercially available from suppliers such as Aldrich Chemical Company of Milwaukee Wis., or can in many cases readily synthesized by the methods of the prior art which are well known to those of ordinary skill in the art. In the presence of bases such as barium hydroxide, methyl ketones comprising the desirable substituents R₃₀ and R₄₀ condense with the benzaldehydes of Formula (500), to form the α,β-unsaturated ketone (501), whose carbonyl group can be selectively reduced with sodium borohydride to form allyl alcohol (502). The carbon-carbon double bond of allyl alcohol (502) can be selectively hydrogenated over palladium/carbon without objectionable levels of isomerization or loss of the hydroxy group, to yield alcohol (503), which can be dehydrated/cyclized in the presence of polyphosphoric acid (PPA) to give the substituted tetrahydronaphthalene (504).

Tetrahydronaphthalene (504) can be selectively oxidized at the open benzylic position with reagents such as chromium trioxide to give the 4,4,6-trisubstituted-3,4-dihydro-2H-naphthalen-1-one of formula (505), which can be readily brominated to give the brominated dihydro-2H-naphthalen-1-one compound (506). The ketone group of compound (506) can be reacted with organometallics such as a desirably substituted alkyl or aryl Grignard or lithium reagent to introduce many desirable R₁₀ groups and form compounds of Formula (507), whose tertiary alcohol group can eliminate in the presence of acid catalysts to form a desirable brominated dihydronapthalene (508). Compound (508) is a valuable precursor of the (Ar_{1g}) radicals of the compounds of Formula (I), which can be converted to a boronic ester suitable for a “normal” Suzuki coupling with carbonyl containing precursors of AR₂, or it can be reacted with bromoesters of AR₂ via a “reverse” Suzuki coupling.

Along similar lines, as shown in FIG. 11, the brominated dihydro-2H-naphthalen-1-one compound (506) can be subjected to a “reverse” Suzuki coupling to yield biaryls of Formula (510), which can be treated with triflic anhydride to form an enol triflate ester of Formula (511), which can be subjected to yet another Suzuki coupling reaction to introduce an aryl or heteroaryl “R₁₀” substituent to the double bond, and form the biaryl compound (512), which can then be condensed with an HAr precursor to form compounds of Formula (I) comprising (Ar_{1g}) radicals.

As is also shown in FIG. 11, substituted benzenes of Formula (513) can be elaborated to provide compounds such as (517), which are precursors of (Ar₁ₕ) radicals for compounds of Formula (I). Substituted benzenes of Formula (513) can be condensed with 2,2,5,5-Tetrasubstituted-dihydro-furan-3-ones in the presence of lewis acids, in analogy to the reactions taught by Barclay, L. R. C. et al, Can. J. Chem. 1970, 48, 2763-2764, to form the ,4-dihydro-1H-naphthalen-2-one compound (514), which can be selectively brominated to form compound (515), which is then reacted with tosylhydrazine to form hydrazone compound (516), whose hydrazone group can be removed by reactions analogous to the reactions taught by Faul M. M. et al, J. Org. Chem., 2001, 66, 5772-5782, to form compound (517), which is a precursor of compounds of Formula (I) having (Ar₁ₕ) radicals.

In view of the teachings and disclosure above, in some aspects, the invention relates to methods for preparing the compounds of Formula (Iₐ), wherein the method comprises
a) coupling
   i) an Ar₁ precursor compound having the structure 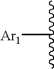
   ii) with an AR₂ precursor compound having the structure 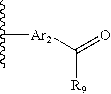
   iii) to form a carbonyl containing precursor compound having the structure 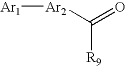
b) further reacting the carbonyl containing precursor compound so as to connect to the carbonyl of the carbonyl containing precursor an HAr heterocycle, to form a compound of Formula (Iₐ).

The methods of making the compounds of the invention further comprise steps wherein the further reacting comprises condensing the carbonyl containing precursor compound with a compound having the structure

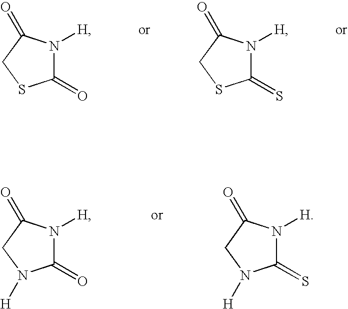

In additional embodiments of the above described methods of making the compounds of the invention, compounds of Formula (Iₐ) are reduced to form compounds of Formula (I_{b}).

As is understood by those of ordinary skill in the art of synthetic organic chemistry, the various synthetic strategies, organic reactions, and/or functional group transformations utilized herein can be performed by a number of strategies, reactions, or procedures other than those explicitly described above. Many such methods of synthesis have been applied to the synthesis of retinoid analog compounds, as is described at length by Dawson et al. in “The Synthetic Chemistry of Retinoids,” Biology, Chemistry, and Medicine, 2nd Edition, Raven Press, Ltd., New York (1994), the entire contents of which are hereby incorporated herein by reference, for the purposes of their teachings regarding the synthesis of compounds related to those described herein and synthetic precursors of the instant compounds.

References for other general synthetic procedures that can be utilized for the synthetic steps leading to the compounds disclosed herein can be found in, for example, March, J., Advanced Organic Chemistry, 4^{th} Edition, Weiley-Interscience (1992); or Larock, R. C., Comprehensive Organic Transformations, A Guide to Functional Group Preparations, VCH Publishers, Inc. (1989), both incorporated herein by reference.

Alternative methods of synthesis of some of the 1-substituted pyrazole compounds of the invention are shown in the reaction schemes below. 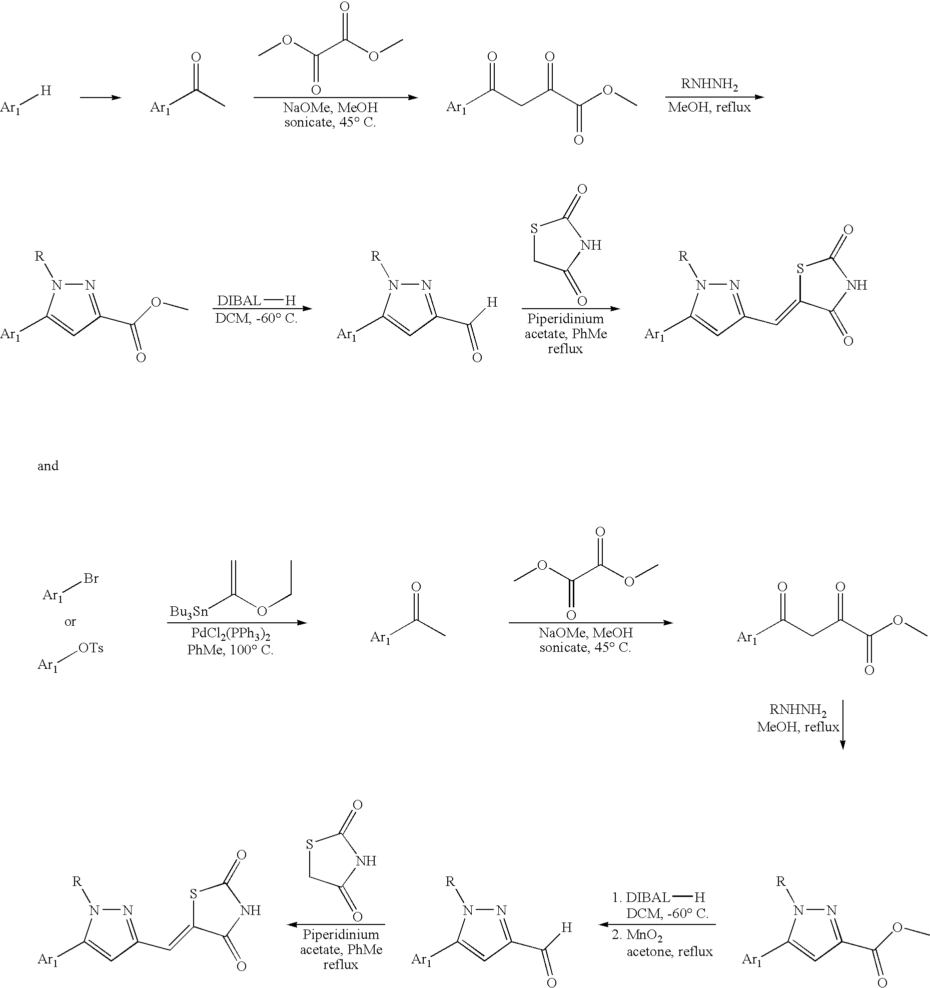
Pharmaceutical Compositions

Although the compounds of Formula (I) described herein can be administered as pure chemicals, it can be preferable to administer the compounds of Formula (I) in the form of a pharmaceutical composition. Thus another embodiment of the invention relates to a pharmaceutical composition comprising one or more compounds of Formula (I), and/or a pharmaceutically acceptable salt thereof, together with one or more pharmaceutically acceptable carriers thereof and, optionally, other therapeutic and/or prophylactic ingredients.

The pharmaceutical compositions can comprise other therapeutic and/or prophylactic ingredients, including presently used drugs. For example, when pharmaceutical compositions comprising the compounds of Formula (I) are used as anti-diabetic agents, the composition may, in some embodiments, contain other suitable anti-diabetic agents (for example Avendia, Actos, Metformin).

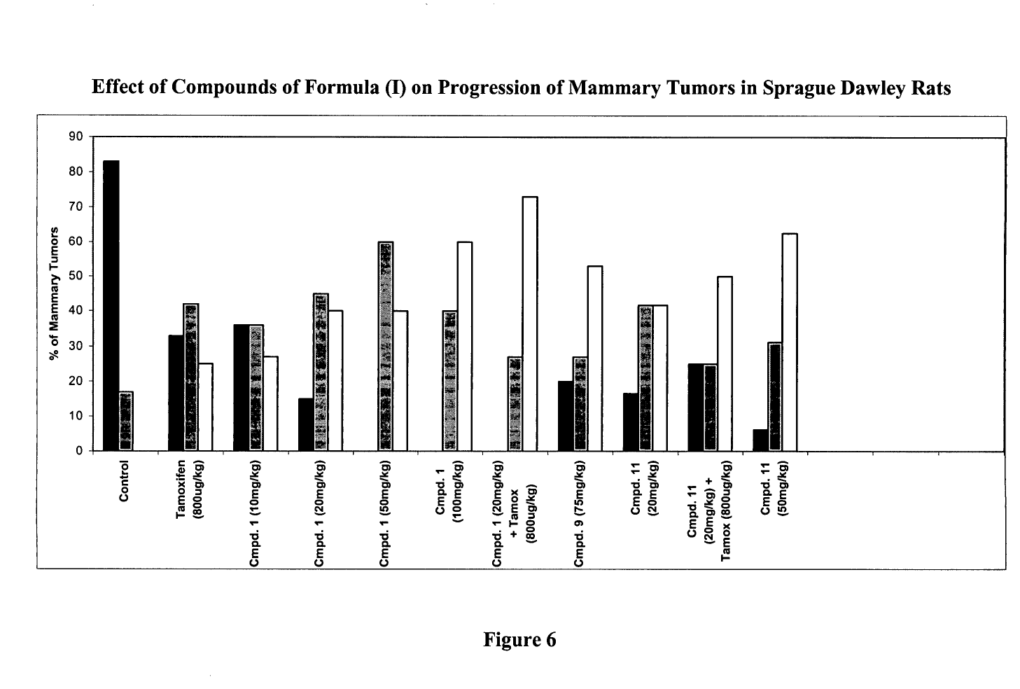

Similarly, when pharmaceutical compositions comprising one or more of the compounds of Formula (I) as an anti-cancer agent, in some embodiments it can be advantageous to include another known and/or presently used anti-cancer agent. For example, as is illustrated in Example 47 and in FIG. 6, unexpectedly improved and/or synergistic effects on the regression of breast cancer tumors were observed when Compound 1 was given in combination with the anti-breast cancer Tamoxifen. Other agents that are effective against breast cancer including Taxol and Taxol derivatives or classical chemotherapeutic agents such as Doxorubicin and Cisplatin may also be suitable to use in combination with herein described molecules for the treatment of breast cancer and other cancers.

The pharmaceutically acceptable carrier(s) are ‘acceptable’ in the sense of being physically and chemically compatible with the other ingredients of the composition and not overly deleterious to the recipient thereof.

Pharmaceutical compositions include those suitable for oral, enteral, parental (including intramuscular, subcutaneous and intravenous), topical, nasal, vaginal, ophthalinical, sublingually or by inhalation administration. The compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

Pharmaceutical compositions suitable for oral administration can be presented as discrete unit dosage forms such as hard or soft gelatin capsules, cachets or tablets each containing a predetermined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art., e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For topical administration to the epidermis, the compounds can be formulated as ointments, creams or lotions, or as the active ingredient of a transdermal patch. Suitable transdermal delivery systems are disclosed, for example, in Fisher et al. (U.S. Pat. No. 4,788,603, incorporated herein by reference) or Bawas et al. (U.S. Pat. Nos. 4,931,279, 4,668,504 and 4,713,224; all incorporated herein by reference). Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. No. 4,140,122, 4,383,529, or 4,051,842; incorporated herein by reference.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof. The pharmaceutical compositions according to the invention can also contain other adjuvants such as flavorings, coloring, antimicrobial agents, or preservatives.

Therefore, in some embodiments the invention relates to a pharmaceutical composition comprising one or more pharmaceutically acceptable carriers and one or more compounds of the invention, or a pharmaceutically acceptable salt thereof, in an amount that can be used to effectively treat diabetes, cancer, or atherosclerosis, or modulate lipid metabolism, carbohydrate metabolism, lipid and carbohydrate metabolism, or adipocyte differentiation, in a mammal.

Biological Activity Testing for Compounds of the Invention

The compounds of Formula (I) and/or their pharmaceutically acceptable salts have been found to be potent compounds in a number of biological assays, both in vitro and in vivo, that correlate to, or are representative of, human diseases.

For instance, many of the compounds of Formula (I) can induce the differentiation of preadipocytes into adipocytes. This biological activity (Harris and Kletzien, Mol. Pharmacol., 45:439-445 (1994); Wilson et al., J. Med. Chem. 39:665-668 (1996)) has been observed for certain compounds that have antidiabetic activity in humans (Teboul et al., J. Biol. Chem. 270:28183-28187 (1995)) and has been used by many in the art to screen new compounds for anti-diabetic activity. The ability of the compounds to induce cells of the adipocyte lineage to differentiate can also correlate to the ability of the compounds to treat or prevent other diseases including proliferative diseases such as breast, prostate and other cancers.

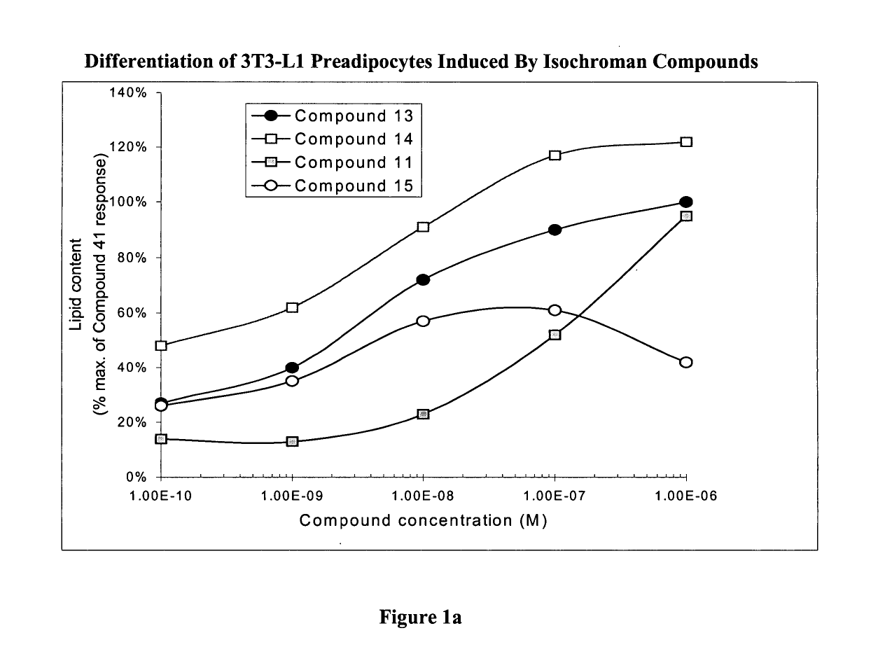

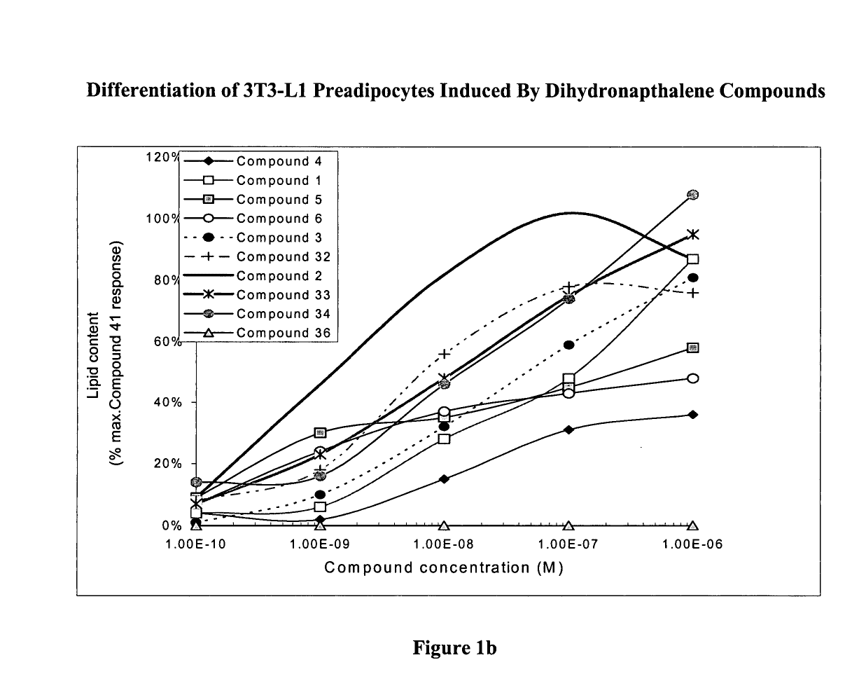

A number of the compounds of Formula (I) have been screened in an in-vitro adipocyte differentiation assay, as described in Example 18. Mouse pre-adipocyte 3T3-L1 cells were treated with compounds at concentrations less than or equal to 10⁻⁶ M for 7 days. Pre-adipocyte cells that become differentiated into adipocytes begin to accumulate lipids, and accordingly can exhibit an increase in lipid content. Results from the testing are shown in FIG. 1a-c, wherein the lipid content of the cells after treatment with the compounds of the invention is displayed as a function of the identity of the compound and the concentration at which it was applied. The relative lipid content of the cells is plotted in FIG. 1a-c relative to the results obtained by the application of comparative compound 41, which has been shown to be a potent inducer of adipocyte differentiation, and also a compound that is useful for the treatment of diabetes.

As can be seen from FIG. 1a-c and/or Example 18, several of the compounds whose preparation is documented in the examples induced differentiation of the pre-adipocytes at concentrations ranging as low as 1×10⁻¹⁰ Molar, and hence showed a positive indication of biological activity.

In order to demonstrate the activity of the various compounds of the invention for effectiveness and/or activity for adipocyte differentiation, the compound can be applied at a concentration of about 1×10⁻⁶ M for a period of about 7 days, to mouse preadipocyte 3T3-L1 cells, and measure the increase the lipid content of the cells. The compounds can be considered active for adipocyte differentiation if the lipid accumulation induced is at least about 20%, or at least about 40% of the lipid accumulation induced by 5-[3-(3,5,5,8,8-Pentamethyl-5,6,7,8-tetrahydro-naphthalen-2-yl)-4-trifluoromethoxy-benzylidene]-thiazolidine-2,4-dione when it is applied to control cultures of mouse preadipocyte 3T3-L1 cells at a concentration of about 1×10⁻⁷ M.

The ability of the compounds to function as antidiabetic agents can be demonstrated in-vivo in certain known animal models for type 2 diabetes [Coleman, D. L, Diabetes, vol. 31, suppl 1, pp 1-6, (1982); Chang A. Y. et al, diabetes, pp 466-470, (1986)]. These known animal models include among others, db/db mice, ob/ob mice, and KKA^{y} mice.

Diabetes and Lipid Metabolism Efficacy Testing in KKA^{y} Mice.

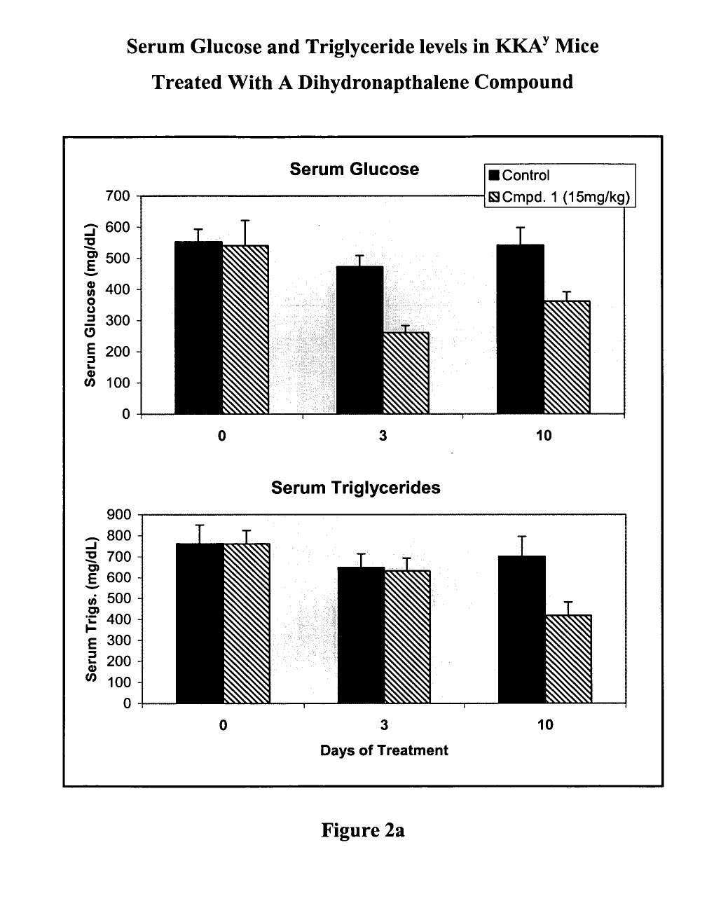

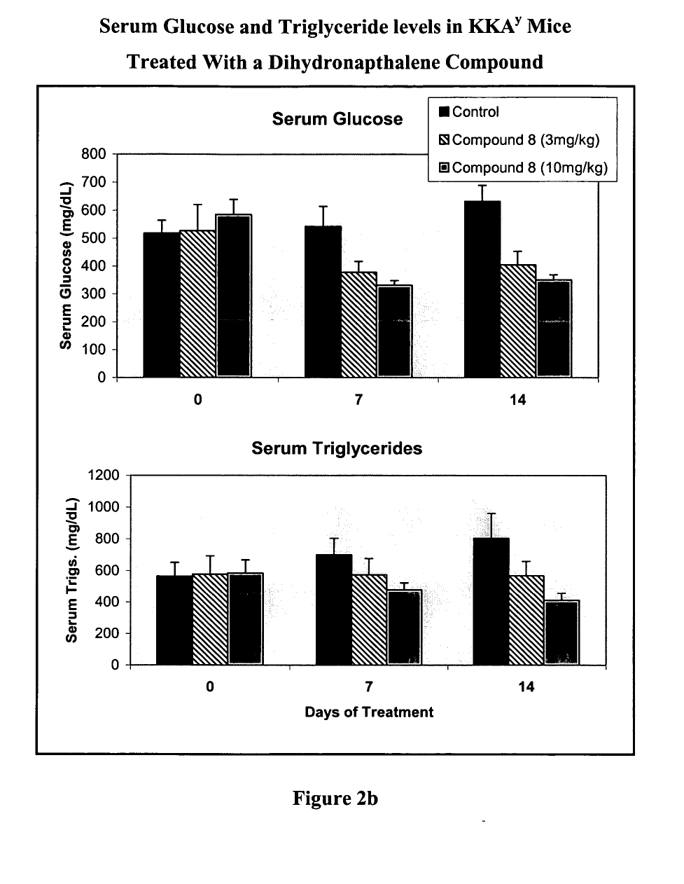

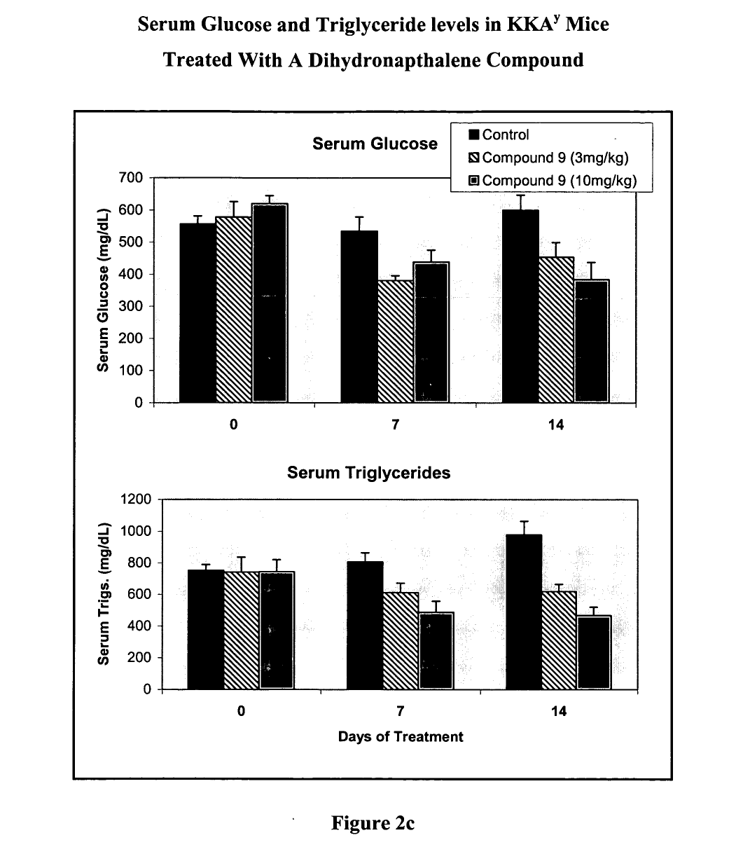

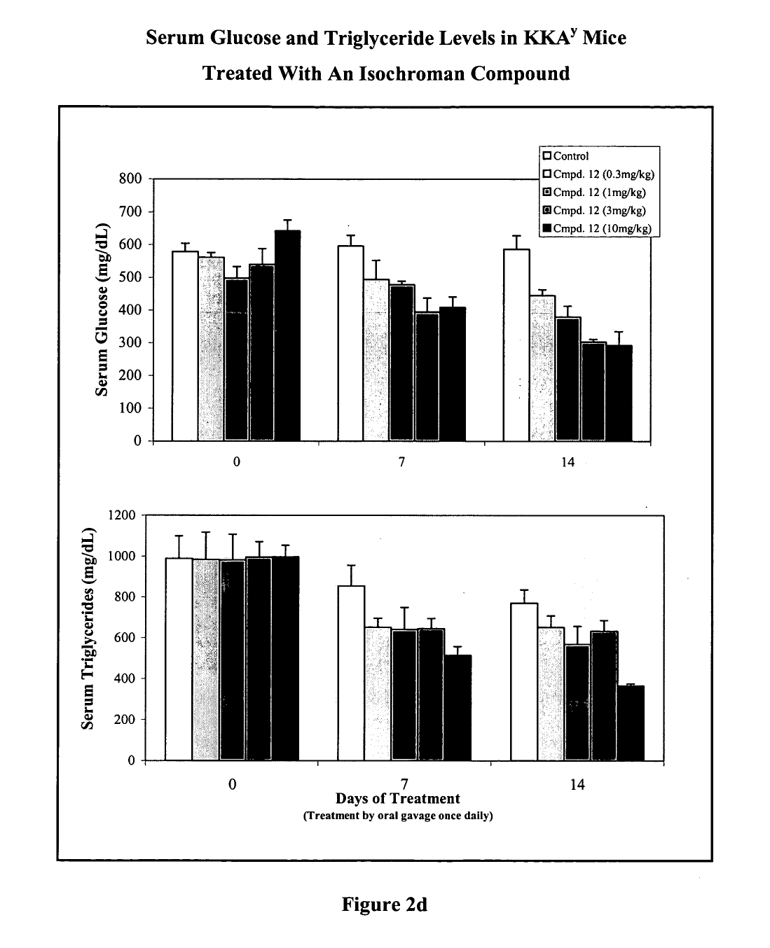

(See Results in FIGS. 2a-d and Example 19.)

Of the three mouse models, the KKA^{y} mice exhibit the most severe symptoms of type 2 diabetes, including hyperglycemia, hypertriglyceridemia and hypercholesterolemia, and therefore are often the most difficult to treat. As can be readily seen from FIGS. 2a-2d, the compounds of the invention were found to be effective for simultaneously and beneficially decreasing serum glucose and serum triglyceride in KKA^{y} Mice.

Activity for Inducing Cholesterol Efflux from Macrophage Foam Cells

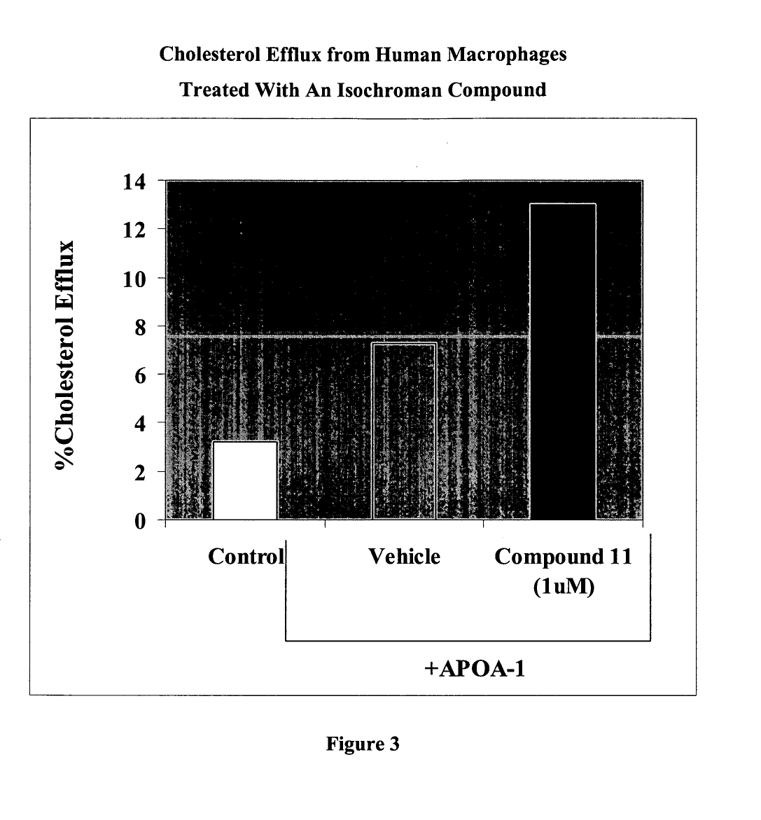

(See Results in FIG. 3 and Example 20)

Elevated levels of cholesterol lead to atherosclerosis and heart disease, which in many type 2 diabetes patients is the cause of death. Atherosclerotic lesions comprise cholesterol-loaded macrophage foam cells [Gown et al. (1986) Am. J. Phathol. 125, 191-207]. In vitro, macrophages that are cholesterol-loaded in cell culture can under some circumstances be induced to unload excess cholesterol, which can be measured in a “Cholesterol Efflux Assay” (see example 20). The cholesterol released from the Macrophage Foam Cells can be metabolized by the liver and eliminated from the body. Therefore, novel therapeutic agents that increase cholesterol efflux from macrophages in arteriosclerotic lesions can improve the outcome for patients with coronary artery disease such as in obese and diabetes patients.

As can be readily seen from FIG. 3, Compound 11 was found to be effective for inducing cholesterol efflux from Macrophage Foam Cells, this indicating its utility for the control and/or treatment of atherosclerosis.

Activity for Modulation of HDL and LDL Cholesterol Levels in Diet Induced Hypercholesterolemic Sprague Dawley Rats

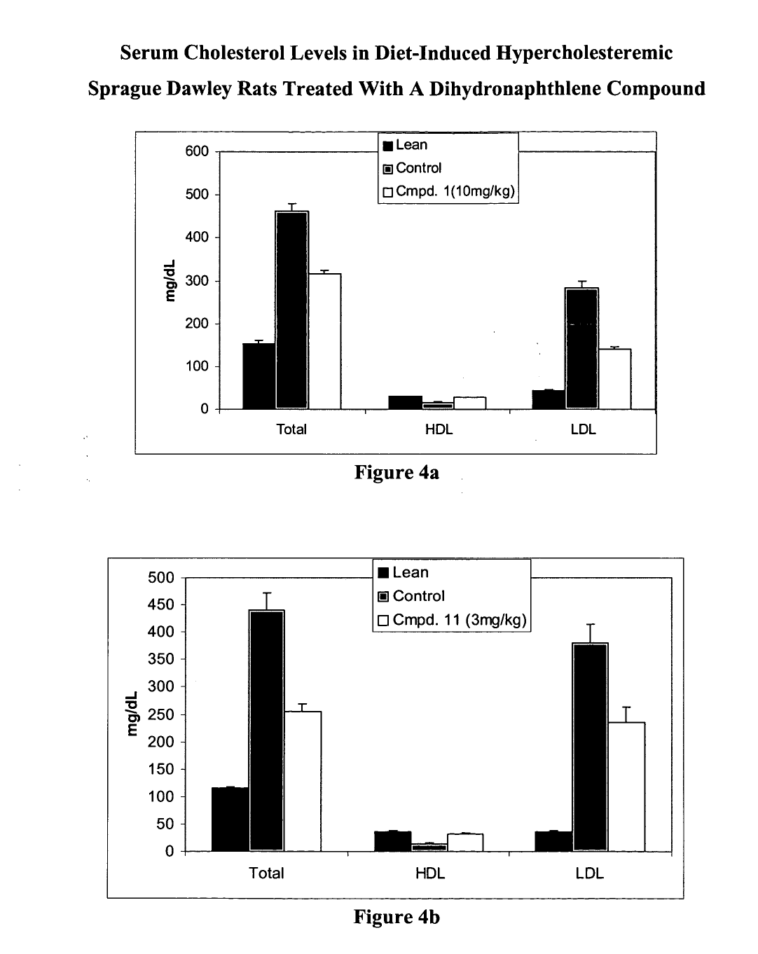

(See Results in FIG. 4a-b and Example 21.)

The ability of a compound to reduce certain lipids such as cholesterol or to change the ratio of good versus bad cholesterol, i.e. HDL versus LDL, can be measured in animal models. One animal model commonly used for such testing is the diet-induced hypercholesterolemic wild type Sprague Dawley rat (see example 21).

As can be readily seen from FIG. 4a-b, Compounds were found to have favorable activity for the modulation of HDL and LDL cholesterol levels in diet-induced hypercholeterolemic Sprague Dawley Rats, thus indicating significant potential for the control and/or treatment of atherosclerosis in human diabetes patients, especially in view of the unexpectedly superior bioavailability of the compounds of Formula (I) as compared to many prior art compounds.

Anti-Cancer Activity

The biological activity of the compounds of Formula (I) can be assayed by testing the compounds or their pharmaceutical compositions for their ability to kill or inhibit the growth of a panel of different human tumor cell lines. Tumor cell lines that can be employed for such tests include but are not limited to known cell lines such as:
For Leukemia: CCRF-CEM, HL-60 (TB), K-562, MOLT-4, RPMI-8226, and SR. Lung Cancer: A549/ATCC, EKVX, HOP-62, HOP-92, NCI-H226, NCI-H23, NCI-H322M, NCI-H460, and NCI-H522.
Colon Cancer: COLO 205, HCC-2998, HCT-116, HCT-15, HT-29, KM-12, and SW-620.
CNS Cancer: SF-268, SF-295, SF-539, SNB-19, SNB-75, and U-251.
Melanoma: LOX-IMVI, MALME-3M, M-14, SK-MEL-2, SK-MEL-28, SK-MEL-5, UACC-257, and UACC-62.
Ovarian Cancer: IGR-OVI, OVCAR-3, OVCAR-4, OVCAR-5, OVCAR-8, and SK-OV-3.
Renal Cancer: 786-0, A-498, ACHN, CAKI-1, RXF-393, RXF-631, SN12C, TK-10, and UO-31.
Prostate Cancer: PC-3 and DU-145.
Breast Cancer: MCF 7, MCF7/ADR-RES, MDA-MB-231/ATCC, HS578T, MDA-MB-435, MDA-N, BT-549, and T-47D.

These anti-cancer activity screening assays provide data regarding the general cytotoxicity of an individual compound. In particular, as described in the examples herein, active anticancer compounds can be identified by applying one or more of the compounds at a concentration of about 10 uM to one or more human tumor cell line cultures, such as for example leukemia, lung cancer, colon cancer, CNS cancer, melanoma, ovarian cancer, renal cancer, prostate cancer, breast cancer, or pancreatic cancer, so as to inhibit cell growth of the tumor cells. In some embodiments, the compounds of Formula (I) are considered to be active for the treatment of cancer if, when they are applied to a culture of one of the above cancer cell lines at a concentration of about 10 uM, for a period of at least about 5 days, the growth of the cancer cells is inhibited, or the cancers cells killed to the extent of about 50% or more, as compared to a control not comprising the compound of the invention.

Effect on Downregulation of Cyclin D1 in MCF-7 Breast Cancer Cells by Selected Compounds

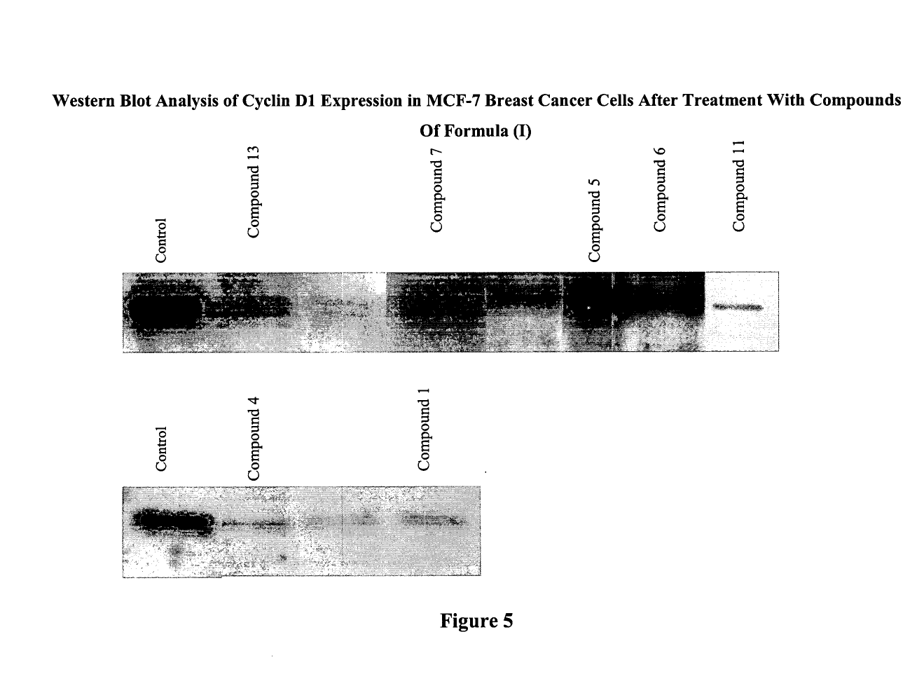

(See Results in FIG. 5 and Example 22.)

Over 50% of human breast cancers overexpress cyclin D1, a 265 amino acid protein component of the core cell-cycle machinery, and several lines of evidence suggest that this overexpression may have a causative role in cancer formation (Xiong Y, Connolly T, Futcher B and Beach D, Cell 1991, 65:691-9). As shown in FIG. 5 a number of compounds of Formula (I) downregulate cylin D1 expression in MCF-7 breast cancer cells, thus

Effect on Breast Cancer Tumor Progression Carcinogen Induced Mammary Tumors in Wild Type Sprague Dawley Rats

(See Results in FIG. 6 and Example 23.)

The ability of the compounds of Formula (I) to function as anti-breast cancer agents can be demonstrated in vivo in carcinogen induced mammary tumors in wild type Sprague Dawley Rats [Thompson H. J et al, Carcinogenesis, 13(9), 1535-1539 (1992)].

As can be readily seen from FIG. 6, Compounds were unexpectedly superior in slowing or causing regression in the growth of breast cancer tumors in Sprague Dawley Rats, thus suggesting utility for the control and/or treatment of breast cancer in humans.

Methods of Treating Diseases

The compounds of Formula (I) disclosed herein, and related prodrugs, and pharmaceutical compositions comprising those compounds or their pharmaceutically acceptable salt, are useful, for example, to modulate metabolism (such as, for example, lipid metabolism and carbohydrate metabolism) or adipocyte differentiation. Changes in carbohydrate metabolism can directly or indirectly also result in changes of lipid metabolism and, similarly, changes in lipid metabolism can lead to changes in carbohydrate metabolism. An example is type 2 diabetes wherein an increase in free fatty acids in the patients leads to decreased cellular uptake and metabolism of glucose.

Carbohydrate metabolism can be up-regulated or down-regulated to either approach the level of carbohydrate metabolism in a control or to deviate from the level of carbohydrate metabolism in a control. For example, the compounds of the invention in conjunction with other unexpedtedly beneficial properties can be effective to lower serum glucose levels of KKA^{y} or db/db mice maintained on a high fat diet by at least about 5%, or at least about 10%, 15%, 20%, 25%, 30%, 40% or 50% when orally administered to the mice at a concentration of about 0.3 to 10, or 15 mg/kg for 7 days, as compared to control mice that do not receive the compounds.

As a result of their activity for regulating carbohydrate metabolism, the compounds of the invention can be effective for treating type 2 diabetes. Therefore, in some embodiments, the invention relates to methods of treating type 2 diabetes comprising administering to a mammal diagnosed as needing such treatment, including humans, one or more compounds of the invention, or a pharmaceutically acceptable salt thereof, in an amount effective to treat type 2 diabetes. In some embodiments, the one or more compounds or salts are applied in an amount effective to decrease blood glucose levels in the mammal by at least about 5%, or at least about 10%, 15%, 20%, 25%, 30%, 40% or 50%.

Modulation of lipid metabolism, for example, can include an increase of lipid content intracellularly or extracellularly. Modulation, for example, could involve increase in lipid metabolism, such that lipid metabolism is greater than that of a control. Modulation, also includes, for example, an increase in lipid metabolism, such that the lipid metabolism approaches that of a control. For example, the compounds of the invention and their pharmaceutically acceptable salts can be employed to induce cholesterol efflux from Macrophage Foam Cells as described in Example 20, in order to treat or prevent atherosclerosis.

Modulation of lipid metabolism could also include a decrease of lipid content intracellularly or extracellularly. Modulation of metabolism can occur directly for example, through binding of the compound of the invention with its cognate receptor, which directly affects an increase or decrease in lipid content by up-regulation or down-regulation of a gene involved in lipid metabolism. Modulation of metabolism can also occur indirectly, for example, through binding of the compound of the invention with its cognate receptor, which up-regulates or down-regulates cellular differentiation or growth of cells that produce lipids, thereby indirectly causing lipid metabolism to be modulated. As shown in Example 21 the compounds of the invention can be effective to lower serum triglyceride levels of KKA^{y} mice maintained on a high fat diet by at least about 5%, or at least about 10%, 15%, 20%, 25%, 30%, 40% or 50%, when orally administered to the mice at a concentration of about 0.3 to 10, or 15 mg/kg for 7 days, as compared to control mice that do not receive the compounds.

Therefore, in some embodiments, the invention relates to methods of treating dyslipidemia comprising administering to a mammal diagnosed as needing such treatment one or more compounds of the invention, or a pharmaceutically acceptable salt thereof, in an amount effective to decrease triglyceride levels in the animal. In some such embodiments, the invention relates to such methods wherein the one or more compounds or salts are applied in an amount effective to decrease triglyceride levels by at least about 5%, or at least about 10%, 15%, 20%, 25%, 30%, 40% or 50%.

Cholesterol is a lipid that is closely linked with many biochemical functions, but also with diseases such as atherosclerosis. As is illustrated in Examples 20 and 21, the compounds of the invention can benefit modulate the level of cholesterol, including its manifestations in the HDL and LDL forms. Therefore, in some embodiments, the invention relates to a method of treating hypercholesterolemia comprising administering to a mammal diagnosed as needing such treatment one or more compounds the invention, or a pharmaceutically acceptable salt thereof. In some embodiments, the methods apply the one or more compounds or salts in an amount effective to decrease serum cholesterol levels by at least about 5%, or at least about 10%, 15%, 20%, 25%, 30%, 40% or 50%, or to increase the concentration of HDL cholesterol, or decrease the concentration of LDL cholesterol, or increase the HDL/LDL ratio by at least about 5%, or at least about 10%, 15%, 20%, 25%, 30%, 40% or 50%.

It is understood that a variety of lipid molecules can be modulated. The compounds disclosed herein can modulate a single type of lipid molecule, such as a triglyceride, or the compounds disclosed herein can modulate multiple types of lipid molecules. The compounds disclosed herein can also modulate a single or variety of carbohydrate molecules. The compounds of the invention can simultaneously and beneficially regulate carbohydrate and lipid metabolism so as to simultaneously decrease levels of serum glucose, serum triglycerides, and serum cholesterol to a superior level. Drugs having such an unexpectedly superior combination of beneficial properties are of very high value for simultaneous treatment of type 2 diabetes and/or its associated diseases, such as atherosclerosis.

Compounds of the invention are also useful for inducing adipocyte differentiation, which can produce a modulation of the metabolism of lipids, including triglycerides and cholesterol. As is shown in Example 18, the compounds of the invention can be effective, when applied at a concentration of about 1 uM for a period of about 7 days, to induce differentiation of mouse preadipocyte 3T3-L1 cells so as to increase their lipid content by at least about 20%, or at least about 40%, or at least about 50%. Such activity for adipocyte differentiation is well known to those of skill in the art to be associated with activity for the treatment of diabetes, cancer, and/or inflammatory diseases. Macrophage foam cells are known to be involved in the formation of atherosclerotic lesions. Compounds of the invention can be involved in lessening such atherosclerotic lesions responses, and/or inducing the macrophages to increase their release of cholesterol, so as to lessen the buildup of cholesterol in blood vessel walls. Therefore, the compounds of the invention are unexpectedly useful in treating diabetes and simultaneously treating the atherosclerosis, which often occurs in diabetic patients.

Compounds of the invention are also useful for treating diseases of uncontrolled cellular proliferation. Compounds can be useful in the treatment of polycystic kidney disease and cancers such as, carcinomas, lymphomas, leukemias, and sarcomas. A representative but non-limiting list of cancers is lymphoma, Hodgkin's Disease, myeloid leukemia, bladder cancer, brain cancer, head and neck cancer, kidney cancer, lung cancers such as small cell lung cancer and non-small cell lung cancer, myeloma, neuroblastoma/glioblastoma, ovarian cancer, pancreatic cancer, prostate cancer, skin cancer, liver cancer, melanoma, colon cancer, cervical carcinoma, breast cancer, and epithelial cancer.

Therefore, in some embodiments, the invention relates to method of treating cancer comprising administering to a mammal diagnosed as needing such treatment one or more compounds of Formula (I), or a pharmaceutically acceptable salt thereof, in an amount effective to treat the cancer. In some embodiments the cancer treated is breast cancer.

Compounds of the invention have desirably low molecular weights to serve as drugs, and good physiological stability. Compounds of the invention also have excellent oral bio-availability, as illustrated in Examples 19, 21, and 23, and FIGS. 2a-2f, and 6, and therefore, represent a class that can have unexpectedly superior pharmacological and physical properties that can be readily implemented to prevent, alleviate, and/or otherwise, treat disorders of lipid and carbohydrate metabolism, such as obesity, dyslipidemia, type 2 diabetes, and other diseases related to type 2 diabetes and diseases of uncontrolled cellular proliferation such as cancer.

A preferred embodiment of the invention relates to the use of the compounds disclosed herein. The compounds disclosed herein can be either used singularly or plurally, and in pharmaceutical compositions thereof for the treatment of mammalian diseases, particularly those related to humans. Compounds disclosed herein and compositions thereof can be administered by various methods including, for example, orally, enterally, parentally, topically, nasally, vaginally, ophthalinically, sublingually or by inhalation for the treatment of diseases related to lipid metabolism, carbohydrate metabolism, lipid and carbohydrate metabolism such as polycystic ovary syndrome, syndrome X, type 2 diabetes, including disorders related to type 2 diabetes such as, diabetic retinopathy, neuropathy, macrovascular disease or differentiation of adipocytes. Routes of administration and dose ages known in the art can be found in Comprehensive Medicinal Chemistry, Volume 5, Hansch, C. Pergamon Press, 1990; incorporated herein by reference.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, one of skill in the art understands how to extrapolate in vivo data obtained in a model organism, such as a KKA^{y} or db/db mouse, to another mammal, such as a human. These extrapolations are not simply based on the weights of the two organisms, but rather incorporate differences in metabolism, differences in pharmacological delivery, and administrative routes. Based on these types of considerations, a suitable dose will, in alternative embodiments, typically be in the range of from about 0.5 to about 100 mg/kg/day, from about 1 to about 75 mg/kg of body weight per day, from about 3 to about 50 mg per kilogram body weight of the recipient per day.

The compound can be conveniently administered in unit dosage form; for example, in alternative embodiments, containing 0.5 to 1000 mg, 5 to 750 mg, most conveniently, or 10 to 500 mg of active ingredient per unit dosage form.

One skilled in the art will recognize that dosage and dosage forms outside these typical ranges can be tested and, where appropriate, be used in the methods of this invention.

In separate embodiments, the active ingredient can be administered to achieve peak plasma concentrations of the active compound of from about 0.5 to about 75 μM, about 1 to 50 μM, or about 2 to about 30 μM. This can be achieved, for example, by the intravenous injection of a 0.05 to 5% solution of the active ingredient, optionally in saline, or orally administered as a bolus containing about 0.5-500 mg of the active ingredient. Desirable blood levels can be maintained by continuous infusion to provide about 0.01-5.0 mg/kg/hr or by intermittent infusions containing about 0.4-15 mg/kg of the active ingredients.

The desired dose can conveniently be presented in a single dose or as divided doses administered at appropriate intervals, for example, as two, three, four or more sub-doses per day. The sub-dose itself can be further divided, e.g., into a number of discrete loosely spaced administrations; such as multiple inhalations from an insufflator or by application of a plurality of drops into the eye.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as can be applied to the essential features hereinbefore set forth, and as follows in the scope of the appended claims.

The following examples are given to illustrate the invention and are not intended to be inclusive in any manner.

### EXAMPLES

### Example 1

### 5[3-(8-Isopropyl-3,5,5-trimethyl-5,6-dihydro-naphthalen-2-yl)-4-trifluoromethoxy-benzylidene]-thiazolidine-2,4-dione, which can be referred to as “Compound 1”

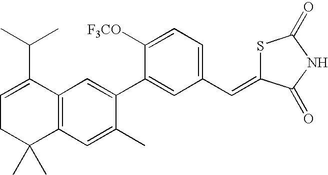

A mixture of toluene (30 mL), piperidine (286 μL), acetic acid (286 μL), 3-[(8-Isopropyl-3,5,5-trimethyl-5,6-dihydro-naphtalene-2-yl)]-4-trifluoromethoxy-benzaldehyde (3.89 g, 9.67 mmol) and 2,4-thiazolidinedione (1.13 g, 9.67 mmol) was heated at reflux overnight and the water was removed using a Dean Stark apparatus. The reaction mixture was cooled to room temperature and the mixture diluted with ethylacetate then washed successively with water and brine, dried over MgSO₄, filtered and evaporated. The residue was chromatographed on silica gel (25% ethyl acetate in hexane) then recrystallized from dichlormethane an hexane to give 3.78 g of 5[3-(8-Isopropyl-3,5,5-trimethyl-5,6-dihydro-naphtalen-2-yl)-4-trifluoromethoxy-benzylidene]-thiazolidine-2,4-dione (70%) yield. mp 179° C. ¹H NMR (300 MHz; DMSO) 1.08 (s, 6H), 1.23 (s, 6H), 2.10 (s, 3H), 2.17 (d, J=4.5 Hz, 2H), 2.89 (m, 1H), 5.77 (t, J=4.2 Hz, 1H), 7.10 (s, 1H), 7.28 (s, 1H), 7.62-7.66 (m, 2H), 7.74 (dd, J=2.4 Hz, J₂=8.7 Hz, 1H), 7.87 (s, 1H), 12.71(s, 1H).

The intermediate 3-[(8-Isopropyl-3,5,5-trimethyl-5,6-dihydro-naphthalene-2-yl)]-4-trifluoromethoxy-benzaldehyde:

a) 3-[(8-Isopropyl-3,5,5-trimethyl-5,6-dihydro-naphtalene-2-yl)]-4-trifluoromethoxy-benzaldehyde.

A mixture of 8-Isopropyl-3,5,5-trimethyl-5,6-dihydro-naphthalene-2-boronic acid (3.65 g, 14.14 mmol), 3-bromo-4-trifluoromethoxy benzaldehyde (3.17 g, 11.78 mmol) and potassium carbonate (3.25 g, 23.56 mmol) in toluene (30 mL), ethanol (6 mL) and water (4.5 mL) was degassed with argon for 30 minutes. Tetrakis(triphenylphosphine)palladium(0) (0.681 g, 0.59 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: 5% ethyl acetate in hexane) to give 3.9 g of 3-[(8-Isopropyl-3,5,5-trimethyl-5,6-dihydro-naphtalene-2-yl)]-4-trifluoromethoxy-benzaldehyde (82%). ¹H NMR (300 MHz;CDCl₃): 1.12 and 1.14 (2 brs, 6H), 1.27 (s, 6H), 2.12 (s, 3H), 2.22 (d, J=4.8 Hz, 2H), 2.88 (m, 1H), 5.78 (t, J=4.5 Hz, 1H), 7.10 (s, 1H), 7.21 (s, 1H), 7.52 (dd, J=1.2 Hz, J₂=8.7 Hz, 1H), 7.85 (d, J=2.4 Hz, 1H), 7.94 (dd, J₁=2.4 Hz, J₂=8.1 Hz, 1H), 10.03 (s, 1H).

b) 8-Isopropyl-3,5,5-trimethyl-5,6-dihydro-naphthalene-2-boronic acid.

To a mixture of 6-bromo-4-isopropyl-1,1,7-trimethyl-1,2-dihydronaphtalene (3.44 g, 11.73 mmol) in THF (30 mL) cooled to −78° C. under an atmosphere of argon was added n-BuLi (7 mL, 2.5 M, 17.66 mmol) dropwise. The resulting suspension was stirred for 5 minutes and triisopropylborate (8 mL, 35.19 mmol) was added dropwise via syringe. The mixture was stirred at −50° C. for 2 hours then warmed up to room temperature and stirred overnight at room temperature. 1.0 N HCl (20 mL) was slowly added to the reaction mixture. After 20 minutes the mixture was diluted with ethyl acetate and the layers separated, the aqueous layer was extracted once with ethyl acetate and the two organic layers combined. The resulting organic layer was washed with water, brine and dried (MgSO₄). The mixture was filtered, evaporated and the residue stirred in hexane. The resulting white suspension was filtered and the white solid dried under high vacuum to afford 2.28 g of 8-Isopropyl-3,5,5-trimethyl-5,6-dihydro-naphtalene-2-boronic acid (75%). %). ¹H NMR (300 MHz;CDCl₃): 1.16 (s, 3H), 1.18 (s, 3H), 1.25 (s, 6H), 2.21 (d, J=4.5 Hz, 2H), 2.86 (s, 1H), 3.09 (m, 1H), 5.78 (t, J=4.5 Hz, 1H), 7.24 (s, 1H), 8.26 (s, 1H).

c) 6-bromo-4-isopropyl-1,1,7-trimethyl-1,2-dihydronaphtalene.

To a cooled (0° C.) solution of isopropyl magnesium chloride (65.5 mL, 2M in ether, 131.01 mmol) was added dropwise a solution of 7-bromo-4,4,6-trimethyl-3,4-dihydro-2H-naphthalen-1-one (7 g, 26.20 mmol) in dry ether (50 mL). The reaction mixture was warmed up to room temperature and stirred overnight. The solution was cooled to 0° C. and acetic acid (10%, 210 mL) was carefully added. The layers were separated and the aqueous was extracted with ether, the combined organic was washed with water, aq. NaHCO₃, brine and dried (MgSO₄), filtered and evaporated to give 7.84 g of crude 7-bromo-1-isopropyl-4,4,6-trimethyl-1,2,3,4-tetrahydronaphthalen-1-ol use as this in the next step.

A mixture of 7-bromo-1-isopropyl-4,4,6-trimethyl-1,2,3,4-tetrahydronaphthalen-1-ol (7.84 g, 25.15 mmol) and p-toluenesulfonic acid (335 mg, 1.76 mmol) in dry MeOH (60 mL) was heated at reflux for 3 hours. After cooling to room temperature the solvent was removed under reduced pressure and the residue chromatographed on silica gel (3 to 10% ethyl acetate in hexane) to give 3.45 g of 6-bromo-4-isopropyl-1,1,7-trimethyl-1,2-dihydronaphtalene (45% overall yield) and recovered 1.58 g of unreacted 7-bromo-4,4,6-trimethyl-3,4-dihydro-2H-naphtalen-1-one. ¹H NMR (300 MHz; CDCl₃): 1.13 (s, 3H), 1.15 (s, 3H), 1.20 (s, 6H), 2.15 (dd, J₁=1.5 Hz, J₂=4.8 Hz, 2H), 2.37 (s, 1H), 2.87 (m, 1H), 5.75 (t, J=4.8 Hz, 1H), 7.14 (s, 1H), 7.44 (s, 1H).

d) 7-bromo-4,4,6-trimethyl-3,4-dihydro-2H-naphthalen-1-one.

A solution of 4,4,6-trimethyl-3,4-dihydro-2H-naphthalen-1-one (58.0 g, 308 mmol) in dichloromethane (110 mL) was added dropwise at room temperature under argon with vigourous stiring to a suspension of aluminum chloride (82.2 g, 616 mmol) in dichloromethane (110 mL). Bromine (19.1 mL, 370 mmol) was then added slowly. The reaction mixture was stirred for 2 hours then poured into concentrated hydrochloride acid (3N, 1 L) and extracted with dichloromethane. The organic layer was washed with water, aq NaHCO₃, water, brine and dried (MgSO₄). The residue was purified on silica gel (eluent: 5 to 8% ethyl acetate in hexane) to give 47.3 g of 7-bromo-4,4,6-trimethyl-3,4-dihydro-2H-naphtalen-1-one (57%). ¹H NMR (300 MHz; CDCl₃): 1.32 (s, 6H), 1.94 (t, J=6.9 Hz, 2H), 2.38 (s, 3H), 2.64 (t, J=6.6 Hz, 2H), 7.23 (s, 1H), 8.06 (s, 1H).

e) 4,4,6-Trimethyl-3,4-dihydro-2H-naphthalen-1-one.

A solution of chromium(VI)oxide (86 g, 0.861 mol) in acetic acid (400 mL) and water (40 mL) was added dropwise to a stirred solution of 1,1,7-trimethyl-1,2,3,4-tetrahydronaphthalene in acetic acid (70 mL) and the reaction mixture stirred 2.5 hours at room temperature. Isopropanol (5 mL) was added and the whole concentrated in vacuo. The residue was dissolved in hexane and filtered over celite. The organic was washed with water and brine, dried (MgSO₄), filtered and evaporated to give 40.3 g of 4,4,6-trimethyl-3,4-dihydro-2H-naphtalen-1-one and used without further purification in the bromination (step d). ¹H NMR (300 MHz; CDCl₃): 1.36 (s, 6H), 1.98 (t, J=6.9 Hz, 2H), 2.38 (s, 3H), 2.68 (t, J=6.9 Hz, 2H), 7.01 (dd, J₁=0.6 Hz, J₂=7.9 Hz, 1H), 7.19 (d, J=0.6 Hz, 1H), 7.90 (d, J=8.1 Hz, 1H).

f) 1,1,7-Trimethyl-1,2,3,4-tetrahydronaphthalene.

A solution of 2-methyl-5-(p-tolyl)-3-pentanol (74 g, 0.385 mol) in dichloromethane (100 mL) was mixed with polyphosphoric acid (570 g) and the reaction mixture was heated to 60° C. and stirred overnight. After cooling, ice/water was slowly added and the aqueous extracted with dichloromethane. The organic layer was successively washed with water, aq NaHCO₃, water and brine, dried (MgSO₄), filtered and evaporated to give 67 g of 1,1,7-trimethyl-1,2,3,4-tetrahydronaphthalene and used without further purification in the next step (step e). ¹H NMR (300 MHz; CDCl₃): 1.34 (s, 6H), 1.69 (m, 2H), 1.83 (m, 2H), 2.37 (s, 3H), 2.78 (m, 2H), 6.99 (m, 2H), 7.19 (s, 1H).

g) 2-Methyl-5-p-tolyl-3-pentanol.

To a solution of trans-4-methyl-1-p-tolyl-1-penten-3-ol (43.2 g, 0.227 mol) in methanol (35 mL) was added 2 micro-spoon of palladium, 10% on activated carbon and the reaction mixture was hydrogenated overnight at 40 psi. The solution was diluted with ethyl acetate, filtered over celite and evaporated to give 40 g of 2-methyl-5-(p-tolyl)-3-pentanol as a colorless oil and used without further purification in the next step (step f). ¹H NMR (300 MHz; CDCl₃): 0.90 (d, J=6.9 Hz, 6H), 1.70 (m, 4H), 2.31 (s, 3H), 2.62 (m, 1H), 2.78 (m, 1H), 3.39 (m, 1H), 7.09 (s, 4H).

h) trans-4-Methyl-1 (p-tolyl)-1-penten-3-ol.

To a solution of 4-methyl-1-p-tolyl-pent-1-en-3-one (77.0 g, 0.41 mol) in methanol (400 mL) was added slowly under argon sodium borohydride (31 g, 0.82 mol). The reaction was stirred at room temperature overnight and methanol (200 mL) was evaporated. The solution was neutralized with hydrochloric acid (2N), extracted with ethyl acetate. The organic layer was successively washed with water, aqueous NaHCO₃, water and brine, dried (MgSO₄), filtered and evaporated to give 80.4 g of trans-4-methyl-1(p-tolyl)-1-penten-3-ol and used without further purification in the next step (step g). ¹H NMR (300 MHz; CDCl₃): 0.94 (d, J=6.9 Hz, 3H), 0.99 (d, J=6.6 Hz, 3H), 1.83 (m, 1H), 2.33 (s, 3H), 4.01 (br, 1H), 6.16 (dd, J₁=7.0 Hz, J₂=16.0 Hz, 1H), 6.53 (d, J=16.0 Hz, 1H), 7.12 (d, J=7.8 Hz, 1H), 7.28 (d, J=7.8 Hz, 1H).

i) trans-4-Methyl-1-p-tolyl-pent-1-en-3-one.

A solution of p-tolualdehyde (100 mL, 0.848 mmol), 3-methyl-2-butanone (181.5 mL, 1.696 mol) and barium hydroxide (20 g, 0.117 mol) in dry ethanol (300 mL) were mixed and the reaction mixture was heated at reflux for 2.5 hour then at room temperature overnight. After cooling some ethanol was removed under reduced pressure and the solution diluted with ethyl acetate. The organic layer was washed with water and brine, dried (MgSO₄), filtered and evaporated to give 154 g of trans-4-methyl-1-p-tolyl-pent-1-en-3-one used without further purification in the next step (step h). ¹H NMR (300 MHz; CDCl₃): 1.16 (d, J=7.2 Hz, 6H), 2.35 (s, 3H), 2.92 (m, 1H), 6.79 (dd, J=16.0 Hz, 1H), 7.19 (d, J=7.5 Hz, 1H), 7.43 (d, J=8.1 Hz, 1H), 7.28 (d, J=16.2 Hz, 1H).

j) 3-Bromo-4-trifluoromethoxy benzaldehyde.

To a solution of 4-trifluoromethoxybenzaldehyde (150 g, 0.79 mol) in a mixture of TFA (400 mL) and H₂SO₄ (80 mL) was added at 40-45° C. N-bromosuccinimide (281 g, 1.579 mol) in equal portion over 2 hours. The reaction mixture was stirred at 40-45° C. overnight, poured into ice-water and extracted with CH₂Cl₂. The organic layer was washed with water then treated with saturated NaHCO₃ (800 mL) for 30 minutes. The layers were separated and the organic layer further washed with water and brine, dried over MgSO₄, filtered and evaporated. The residue was triturated with hexane and filtered. After evaporation of the solvent, the residue was distilled to give 3-bromo-4-trifluoromethoxybenzaldehyde (150.2 g, 60° C., 0.3 mm/Hg, 70%). ¹H NMR (300 MHz; CDCl₃): 7.49 (dd, J₁=1.8 Hz and J₂=8.7 Hz, 1H), 7.88 (dd, J₁=2.1 Hz and J₂=8.4 Hz, 1H), 8.17 (d, J=1.8 Hz, 1H), 9.97 (s, 1H).

### Example 2

### 5-[4-Dimethylamino-3-(8-isopropyl-3,5,5-trimethyl-5,6-dihydro-naphthalen-2-yl)-benzylidene]-thiazolidine-2,4-dione, which can be referred to as “Compound 2”

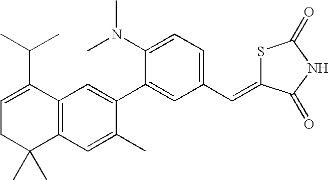

Prepared in a similar manner to example 1 using 4-Dimethylamino-3-(8-isopropyl-3,5,5-trimethyl-5,6-dihydro-naphthalen-2-yl)-benzaldehyde. 80% yield after crystallization from dichloromethane and hexane. mp 231° C. ¹H NMR (300 MHz; DMSO) 1.08 (d, J=6.60 Hz, 3H), 1.12 (d, J=6.60 Hz, 3H), 1.22 (s, 6H), 2.10 (s, 3H), 2.16 (d, J=4.10 Hz, 2H), 2.57 (s, 6H), 2.91 (m, 1H), 5.75 (t, J=4.54 Hz, 1H), 7.09 (d, J=8.50 Hz, 1H), 7.15 (s, 1H), 7.23 (s, 1H), 7.25 (d, J=1.46 Hz, 1H), 7.49 (dd, J₁=1.76 Hz, J₂=8.50 Hz, 1H), 7.74 (s, 1H), 12.44 (m, 1H).

The intermediate 3-(5-Isobutyryl-3,3-dimethyl-2,3-dihydro-benzofuran-7-yl)-4-trifluoromethoxy-benzaldehyde was prepared in a similar manner to example 1a using 8-isopropyl-3,5,5-trimethyl-5,6-dihydro-naphthalene-2-boronic acid (example 1b) and 3-bromo-4-dimethylaminobenzaldehyde.

a) 3-bromo-4-dimethylamino-benzaldehyde.

To a solution of 4-dimethylamino-benzaldehyde (10 g, 67.03 mmol) in dichloromethane (250 mL) was added pyridinium tribromide (21.4 g, 67.03 mmol) and the reaction mixture stirred at room temperature overnight. The solution was washed with water and brine, dried (MgSO₄), filtered and evaporated. The residue was purified on silica gel (eluent: 15% ethyl acetate in hexane) to give 14.06 g of 3-bromo-4-dimethylamino-benzaldehyde (92%). ¹H NMR (300 MHz; CDCl₃); 2.59 (s, 6H), 7.06 (d, J=8.1 Hz, 1H), 7.75 (dd, J₁=7.8 Hz, J₂=1.5 Hz, 1H), 5.74 (s, 1H), 7.06 (d, J=8.1 Hz, 1H), 7.43 (dd, J₁=2.1 Hz, J₂=8.4 Hz, 1H), 8.04 (d, J=1.8 Hz, 1H), 9.81 (s, 1H).

### Example 3

### 5-[2,5-Difluoro-3-(8-isopropyl-3,5,5-trimethyl-5,6-dihydro-naphthalen-2-yl)-4-methoxybenzylidene]-thiazolidine-2,4-dione, which can be referred to as “Compound 3”

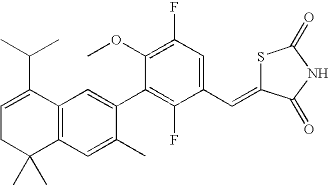

Prepared in a similar manner to example 1 using 2,5-Difluoro-3-(8-isopropyl-3,5,5-trimethyl-5,6-dihydro-naphthalen-2-yl)-4-methoxybenzaldehyde. 51% yield. mp 244° C. ¹H NMR (300 MHz; DMSO) 1.07 (s, 3H), 1.09 (s, 3H), 1.23 (s, 6H), 2.08 (s, 3H), 2.17 (d, J=4.2 Hz, 2H), 2.09 (m, 1H), 3.76 (d, J=2.4 Hz, 3H), 5.76 (t, J=4.2 Hz, 1H), 7.14 (s, 1H), 7.29 (s, 1H), 7.43 (dd, J₁=6.9 Hz, J₂=12.3 Hz, 1H), 7.71(s, 1H), 12.76 (s, 1H).

The intermediate 2,5-Difluoro-3-(8-isopropyl-3,5,5-trimethyl-5,6-dihydro-naphthalen-2-yl)-4-methoxybenzaldehyde was prepared as followed:

a) 2,5-Difluoro-3-(8-isopropyl-3,5,5-trimethyl-5,6-dihydro-naphthalen-2-yl)-4-methoxybenzaldehyde:

To a solution of the 2-[2,5-Difluoro-3-(8-isopropyl-3,5,5-trimethyl-5,6-dihydro-naphthalen-2-yl)-4-methoxy-phenyl]-[1,3]dioxolane (21.2 g, 49.47 mmol) in THF (120 mL), was added HCl (1N, 60 mL). The reaction mixture was warmed up to 45-50° C. and stirred for 3.5 hours. The reaction was cooled to room temperature, and extracted with ethylacetate, washed with water, aq. NaHCO₃, water and brine, dried over MgSO₄, filtered and evaporated. The residue was chromatographed on silica gel (4% ethylacetate in hexane) to give 11.8 g of 2,5-Difluoro-3-(8-isopropyl-3,5,5-trimethyl-5,6-dihydro-naphthalen-2-yl)-4-methoxybenzaldehyde. ¹H NMR (300 MHz; CDCl₃) 1.13 (s, 3H), 1.15 (s, 3H), 1.27 (s, 6H), 2.15 (s, 3H), 2.22 (d, J=4.5 Hz, 2H), 2.89 (m, 1H), 3.86 (d, J=3.0 Hz, 3H), 5.76 (t, J=4.8 Hz, 1H), 7.09 (s, 1H), 7.26 (s, 1H), 7.62 (dd, J₁=6.6 Hz, J₂=11.4 Hz, 1H), 10.26 (d, J=3.0 Hz, 1H).

b) 2-[2,5-Difluoro-3-(8-isopropyl-3,5,5-trimethyl-5,6-dihydro-naphthalen-2-yl)-4-methoxy-phenyl]-[1,3]dioxolane:

The intermediate 2-[2,5-Difluoro-3-(8-isopropyl-3,5,5-trimethyl-5,6-dihydro-naphthalen-2-yl)-4-methoxy-phenyl]-[1,3]dioxolane was prepared in a similar manner to example 1a using 8-isopropyl-3,5,5-trimethyl-5,6-dihydro-naphthalene-2-boronic acid (example 1b) and 3-bromo-2,5-difluoro-4-methoxy benzaldehyde.

c) 3-bromo-2,5-difluoro-4-methoxy benzaldehyde

Hexamethyltetramine (53.88 g, 0.384 mmol) was added carefully to trifluoroacetic acid (TFA, 140 mL) and the solution warmed to 80° C. A solution of 2,5-difluorophenol (25 g, 0.192 mmol) in THF (60 mL) was added dropwise to the reaction mixture and the reaction stirred for 3 hrs at 80° C. The solution was diluted with toluene and the TFA removed under reduced pressure. The solution was then poured into ice-water and extracted with ethylacetate, washed successively with water, saturated aqueous NaHCO₃ (to pH=6), water and brine, dried (MgSO₄), filtered and evaporated to give 17 g of crude 2,5-difluoro-4-hydroxybenzaldehyde use as this in the next step.

To a solution of 2,5-difluoro-4-hydroxybenzaldehyde (37.5 g, 0.237 mmol) in dichloromethane (1.5 L) was added pyridinium tribromide (75.9 g, 0.237 mmol). The reaction mixture was stirred at 40° C. for 7 hrs then at room temperature overnight. The reaction was washed with water and brine, dried over magnesium sulfate, filtered and evaporated to give 48.4 g of crude 3-bromo-2,5-difluoro-4-hydroxybenzaldehyde use as this in the next step.

To a solution of 3-bromo-2,5-difluoro-4-hydroxybenzaldehyde (48.4 g, 0.193 mmol) in DMF (200 mL) was added potassium carbonate (40.0 g) and dimethylsulfate (27.4 mL). The reaction mixture was stirred at room temperature overnight. The reaction was diluted wit ethylacetate and washed successively with water and brine, dried over magnesium sulfate, filtered and evaporated. The residue was triturated with hexane to afford 26 g of 3-bromo-2,5-difluoro-4-methoxy benzaldehyde. The mother liquor was evaporated and chromatographed on silica gel (0-10% ethyl acetate in hexane) to give 10.86 g of more product. (38% overall yield from 2,5-fluorophenol). ¹H NMR (300 MHz; CDCl₃): 4.15 (s, 3H), 7.59 (dd, J₁=6.6 Hz, J₂=11.4 Hz, 1H), 10.22 (d, J=3.3 Hz, 1H).

### Example 4

### 5-[4-Trifluoromethoxy-3-(3,5,5-trimethyl-8-phenyl-5,6-dihydro-naphthalen-2-yl)-benzylidene]-thiazolidine-2,4-dione, which can be referred to as “Compound 4”

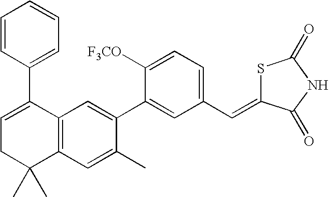

Prepared in a similar manner to example 1 using 4-trifluoromethoxy-3-(3,5,5-trimethyl-8-phenyl-5,6-dihydro-naphthalen-2-yl)-benzaldehyde. mp 249.6° C. ¹H NMR (300 MHz; DMSO) 1.34 (s, 6H); 2.13 (s, 3H); 2.35 (d, J=4.2 Hz, 2H); 6.00 (t, J=4.2 Hz, 1H); 6.70 (s, 1H); 7.28-7.38 (m, 6H); 7.52 (d, J=2.4 Hz, 1H); 7.58 (d, J=7.8 Hz, 1H); 7.69 (dd, J₁=2.1 Hz, J₂=8.7 Hz, 1H); 7.81 (s, 1H); 12.70 (s, 1H).

a) The intermediate 4-trifluoromethoxy-3-(3,5,5-trimethyl-8-phenyl-5,6-dihydro-naphthalen-2-yl)-benzaldehyde was prepared in a similar manner to example 1a using 8-phenyl-3,5,5-trimethyl-5,6-dihydro-naphthalene-2-boronic acid and 3-bromo-4-trifluoromethoxy benzaldehyde (example 1j). ¹H NMR (300 MHz; CDCl₃) 1.34 (s, 6H); 2.13 (s, 3H); 2.35 (d, J=4.2 Hz, 2H); 5.95 (t, J=4.2 Hz, 1H); 6.84 (s, 1H); 7.28-7.38 (m, 6H); 7.52 (dd, J₁=1.8 Hz, J₂=10.2 Hz, 1H); 7.73 (d, J=1.8 Hz, 1H); 7.88 (dd, J₁=1.8 Hz, J₂=8.4 Hz, 1H); 9.96 (s, 1H).

b) 8-phenyl-3,5,5-trimethyl-5,6-dihydro-naphthalene-2-boronic:

Prepared in a similar manner to example 1b using 6-bromo-1,1,7-trimethyl-4-phenyl-1,2-dihydro-naphthalene.

c) 6-bromo-1,1,7-trimethyl-4-phenyl-1,2-dihydro-naphthalene:

To a cooled (−10° C.) solution of 7-bromo-4,4,6-trimethyl-3,4-dihydro-2H-naphthalen-1-one (example 1d) (1.08 g, 4.04 mmol) in dry THF (25 mL) was added dropwise a solution of phenyl magnesium bromide (5.37 mL, 3M in ether, 4 eq). The reaction mixture was stirred at −10° C. for 1 hour and at room temperature for 1 hour. The solution was cooled to 0° C., saturated amonium chloride solution was carefully added and the sokution extracted with ethylacetate. The organic was washed with water, brine and dried (MgSO₄), filtered and evaporated to give 0.96 g of crude 7-bromo-4,4,6-trimethyl-1-phenyl-1,2,3,4-tetrahydronaphthalen-1-ol use as this in the next step.

A mixture of 7-bromo-4,4,6-trimethyl-1-phenyl-1,2,3,4-tetrahydronaphthalen-1-ol (0.96 g) and p-toluenesulfonic acid (56 mg) in dry MeOH (25 mL) was heated at reflux for 5 hours. After cooling to room temperature water was added and the mixture extracted with ethylacetate. The organic was further washed with water and brine, dried (MgSO₄), filtered and evaporated. The residue was chromatographed on silica gel (10% ethyl acetate in hexane) to give 0.577 g of 6-bromo-1,1,7-trimethyl-4-phenyl-1,2-dihydro-naphthalene (63% overall yield). ¹H NMR (300 MHz; CDCl₃): 1.34 (s, 6H), 2.35 (d, J=4.8 Hz, 2H), 2.42 (s, 3H), 5.98 (t, J=4.8 Hz, 1H), 7.20 (s, 1H), 7.24 (s, 1H), 7.34-7.41 (m, 5H).

### Example 5

### 5-[4-Trifluoromethoxy-3-(3,5,5-trimethyl-8-thiophen-2-yl-5,6-dihydro-naphthalen-2-yl)-benzylidene]-thiazolidine-2,4-dione, which can be referred to as “Compound 5”

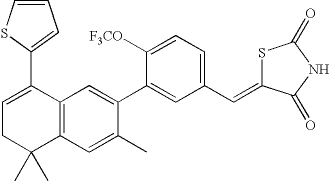

Prepared in a similar manner to example 1 using 4-Trifluoromethoxy-3-(3,5,5-trimethyl-8-thiophen-2-yl-5,6-dihydro-naphthalen-2-yl)-benzaldehyde. mp 250° C. ¹H NMR (300 MHz, DMSO-d-6): 1.31 (s, 6H), 2.13 (s, 3H), 2.34 (d, J=4.1 Hz, 2H), 6.19 (t, J=4.7 Hz, 1H), 7.06 (d, J=4.1 Hz, 2H), 7.07 (s, 1H), 7.38 (s, 1H), 7.47 (t, J=3.5 Hz, 1H), 7.59 (s, 1H), 7.60 (d, J=11.1 Hz, 1H), 7.71 (dd, J₁=8.8 Hz, J₂=2.2 Hz, 1H), 7.84 (s, 1H), 12.70 (br s, 1H).

The intermediate 4-Trifluoromethoxy-3-(3,5,5-trimethyl-8-thiophen-2-yl-5,6-dihydro-naphthalen-2-yl)-benzaldehyde was prepared as followed:

a) 4-Trifluoromethoxy-3-(3,5,5-trimethyl-8-thiophen-2-yl-5,6-dihydro-naphthalen-2-yl)-benzaldehyde:

A solution of trifluoro-methanesulfonic acid 7-(5-[1,3]dioxolan-2-yl-2-trifluoromethoxy-phenyl)-4,4,6-trimethyl-3,4-dihydro-naphthalen-1-yl ester (0.917 g, 1.66 mmol), 2-thiopheneboronic acid (255 mg, 1.99 mmol) and potassium carbonate (459 mg, 3.32 mmol) in a mixture of toluene (5.3 mL), ethanol (1 mL) and water (0.35 mL) was degassed with argon for 30 minutes. Pd(PPh₃)₄ was added and the mixture was refluxed for 17 hours. The reaction was cooled to room temperature, and extracted with ethylacetate, washed with water and brine, dried over MgSO₄, filtered and evaporated. The residue was dissolved in acetone (10 mL) and HCl (1N, 12 mL) was added and the reaction stirred overnight at room temperature. The solution was extracted with dichloromethane and the organic further washed with water, aq. NaHCO₃ and brine, dried over MgSO₄, filtered and evaporated. The residue was chromatographed on silica gel (30% ethylacetate in hexane) to give 0.521 g of 4-Trifluoromethoxy-3-(3,5,5-trimethyl-8-thiophen-2-yl-5,6-dihydro-naphthalen-2-yl)-benzaldehyde (71%). ¹H NMR (300 MHz; CDCl₃) 1.35 (s, 6H), 2.14 (s, 3H), 2.34 (d, J=4.8 Hz, 2H), 6.16 (t, J=4.8 Hz, 1H), 7.09 (m, 2H), 7.18 (m, 2H), 7.24 (s, 1H), 7.44 (dd, J₁=1.8 Hz, J₂=8.4 Hz, 1H), 7.77 (d, J=2.4 Hz, 1H), 7.88 (dd, J₁=2.1 Hz, J₂=8.7 Hz, 1H), 9.97 (s, 1H).

b) Trifluoro-methanesulfonic acid 7-(5-[1,3]dioxolan-2-yl-2-trifluoromethoxy-phenyl)-4,4,6-trimethyl-3,4-dihydro-naphthalen-1-yl ester:

To a cold (−78° C.) solution of sodium bis(trimethylsilyl)amide (1M in THF, 0.76 mL, 0.76 mmol) in THF was added 7-(5-[1,3]dioxolan-2-yl-2-trifluoromethoxy-phenyl)-4,4,6-trimethyl-3,4-dihydro-2H-naphthalen-1-one (267 mg, 0.63 mmol). After stiring at −78° C. for 1 hour, N-phenyltrifluoromethane sulfonimide (272 mg, 0.76 mmol) was added in one portion. After 30 minutes the solution was warmed to 0° C. and stirred overnight. The reaction was quenched by the addition of saturated aqueous ammonioum chloride and extracted with ethylacetate. The organic was washed with a saturated solution of K₂CO₃, brine, dried over MgSO₄, filtered and evaporated. Purification on silica gel (20% ethylacetate in hexane) afforded 320 mg of trifluoro-methanesulfonic acid 7-(5-[1,3]dioxolan-2-yl-2-trifluoromethoxy-phenyl)-4,4,6-trimethyl-3,4-dihydro-naphthalen-1-yl ester (91%). ¹H NMR (300 MHz; CDCl₃) 1.35 (s, 6H), 2.13 (s, 3H), 2.42 (t, J=4.5 Hz, 2H), 4.08 (m, 4H), 5.84 (s, 1H), 5.92 (t, J=4.8 Hz, 1H), 7.16-7.39 (m, 7H), 7.51 (dd, J₁=2.1 Hz, J₂=8.4 Hz, 1H).

c) 7-(5-[1,3]Dioxolan-2-yl-2-trifluoromethoxy-phenyl)-4,4,6-trimethyl-3,4-dihydro-2H-naphthalen-1-one:

To a solution of 4-trifluoromethoxy-3-(3,5,5-trimethyl-8-oxo-5,6,7,8-tetrahydro-naphthalen-2-yl)-benzaldehyde (4.16 g, 11.05 mmol) in toluene (45 mL) was added ethylene glycol (12.3 mL, 221 mmol) and p-toluenesulfonic acid monohydrate (0.126 g, 0.66 mmol). The reaction mixture was heated to reflux overnight, the water was removed using a Dean-Stark apparatus. After cooling, potassium carbonate (10%, 65 mL) was added and the mixture stiired for 30 minutes. The solution was extracted with ethyl acetate, the organic phase was further washed with aq K₂CO₃, brine, dried over MgSO₄, filtered and evaporated. Purification on silica gel (20% ethylacetate in hexane) afforded 4.09 g of 7-(5-[1,3]dioxolan-2-yl-2-trifluoromethoxy-phenyl)-4,4,6-trimethyl-3,4-dihydro-2H-naphthalen-1-one (88%). ¹H NMR (300 MHz; CDCl₃) 1.42 (br s, 6H), 2.05 (m, 2H), 2.18 (s, 3H), 2.73 (t, J=6.9 Hz, 2H), 4.08 (m, 4H), 5.82 (s, 1H), 7.28 (s, 1H), 7.32 (d, J=8.4 Hz, 1H), 7.39 (d, J=2.1 Hz, 1H), 7.51 (dd, J₁=2.1 Hz, J₂=8.4 Hz, 1H), 7.84 (s, 1H).

d) 4-trifluoromethoxy-3-(3,5,5-trimethyl-8-oxo-5,6,7,8-tetrahydro-naphthalen-2-yl)-benzaldehyde.

A mixture of 7-bromo-4,4,6-trimethyl-3,4-dihydro-2H-naphthalen-1-one (example 1d) (3.8 g, 14.25 mmol), 3-formyl-6-trifluoromethoxy-1-phenyl boronic acid (4.0 g, 17.10 mmol) and potassium carbonate (3.9 g, 28.50 mmol) in toluene (40 mL), ethanol (8 mL) and water (6 mL) was degassed with argon for 30 minutes. Tetrakis(triphenylphosphine)palladium(0) (0.33 g, 0.285 mmol) was added and the mixture heated at reflux under argon overnight. The solution was cooled to room temperature, diluted with ethyl acetate and washed successively with water and brine, dried over anhydrous magnesium sulfate, filtered and evaporated. The residue was purified on silica gel (eluent: 10% ethyl acetate in hexane) to give 4.17 g of 4-trifluoromethoxy-3-(3,5,5-trimethyl-8-oxo-5,6,7,8-tetrahydro-naphthalen-2-yl)-benzaldehyde (77%). ¹H NMR (300 MHz; CDCl₃) 1.44 (br s, 6H), 2.05 (br m, 2H), 2.19 (s, 3H), 2.75 (t, J=7.2 Hz, 1H), 7.33 (s, 1H), 7.52 (dd, J₁=8.7 Hz, J₂=1.8 Hz, 1H), 7.81 (dd, J₁=7.2 Hz, J₂=2.1 Hz, 1H),), 7.95 (dd, J₁=8.1 Hz, J₂=1.5 Hz, 1H), 10.02 (s, 1H).

e) 3-formyl-6-trifluoromethoxy-1-phenyl boronic acid.

To a mixture of 2-(3-bromo-4-trifluoromethoxy-1-phenyl)-1,3-dioxolane (7.20 g, 22.9 mmol) in THF (70 mL) cooled to −78° C. under an atmosphere of argon was added n-BuLi (13.8 mL, 2.5 M, 34.4 mmol) dropwise. The resulting suspension was stirred for 5 minutes and triisopropylborate (15.9 mL, 68.7 mmol) was added dropwise via syringe. The mixture was stirred at −50° C. for 2 hours then warmed up to room temperature and stirred overnight at room temperature. 1.0 N HCl (50 mL) was slowly added to the reaction mixture. After 3 hours the mixture was diluted with ethyl acetate and the layers separated, the aqueous layer was extracted once with ethyl acetate and the two organic layers combined. The resulting organic layer was washed with water, brine and dried (MgSO₄). The mixture was filtered, evaporated and the residue stirred in hexane. The resulting white suspension was filtered and the white solid dried under high vacuum to afford 3.00 g of 3-formyl-6-trifluoromethoxy-1-phenyl boronic acid (56%). ¹H NMR (300 MHz; CDCl₃): 7.42 (d, J=7.0 Hz, 1H), 8.07 (dd, J₁=2.1 Hz, J₂=8.7 Hz, 1H), 8.47 (d, J=1.8 Hz, 1H), 10.05 (s, 1H).

f) 2-(3-bromo-4-trifluoromethoxy-1-phenyl)-1,3-dioxolane.

To a solution of 3-bromo-4-trifluoromethoxybenzaldehyde (example 1j) (20 g, 74.0 mmol) in toluene (200 mL) was added ethylene glycol (82.6 mL, 1.48 mol) and p-toluenesulfonic acid monohydrate (0.84 g, 4.44 mmol). The reaction mixture was heated at reflux overnight and the water was removed using a Dean Stark apparatus. The solution was cooled to room temperature, poured into aqueous potassium carbonate (10%) and extracted with ethyl acetate. The organic layer was washed with water, brine and dried (MgSO₄). The residue was purified on silica gel (eluent: 10% ethyl acetate in hexane) to give 15.4 g of 2-(3-bromo-4-trifluoromethoxy)-1,3-dioxolane (66%). ¹H NMR (500 MHz; CDCl₃): 4.05 (m, 2H), 4.11 (m, 2H), 5.79 (s, 1H), 7.32 (d, 1H), 7.43 (d, 1H), 7.77 (d, J=1.1 Hz, 1H).

### Example 6

### 5-[4-Trifluoromethoxy-3-(3,5,5-trimethyl-8-thiophen-3-yl-5,6-dihydro-naphthalen-2-yl)-benzylidene]-thiazolidine-2,4-dione, which can be referred to as “Compound 6”

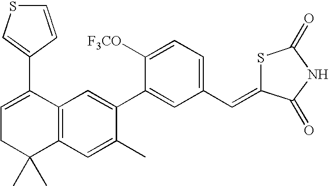

Prepared in a similar manner to example 1 using 4-Trifluoromethoxy-3-(3,5,5-trimethyl-8-thiophen-3-yl-5,6-dihydro-naphthalen-2-yl)-benzaldehyde. mp 244° C. ¹H NMR (300 MHz, DMSO-d-6): 1.32 (s, 6H), 2.12 (s, 3H), 2.32 (d, J=4.1 Hz, 2H), 6.11 (t, J=4.6 Hz, 1H), 6.87 (s, 1H), 7.09 (dd, J₁=5.0 Hz, J₂=1.2 Hz, 1H), 7.36 (s, 1H), 7.44 (d, J=1.8 Hz, 1H), 7.55 (dd, J₁=3.2 Hz, J₂=5.0 Hz, 1H), 7.59 (s, 1H), 7.60 (d, J=10.2 Hz, 1H), 7.70 (dd, J₁=8.8 Hz, J₂=2.2 Hz, 1H), 7.83 (s, 1H), 12.70 (br s, 1H).

The intermediate 4-Trifluoromethoxy-3-(3,5,5-trimethyl-8-thiophen-3-yl-5,6-dihydro-naphthalen-2-yl)-benzaldehyde was prepared in a similar manner to example 5a using trifluoro-methanesulfonic acid 7-(5-[1,3]dioxolan-2-yl-2-trifluoromethoxy-phenyl)-4,4,6-trimethyl-3,4-dihydro-naphthalen-1-yl ester (example 5b) and 3-thiophene boronic acid.

### Example 7

### 5-[4-Trifluoromethoxy-3-(3,5,5-trimethyl-8-thiophen-2-yl-5,6-dihydro-naphthalen-2-yl)-benzylidene]-thiazolidine-2,4-dione, which can be referred to as “Compound 7”

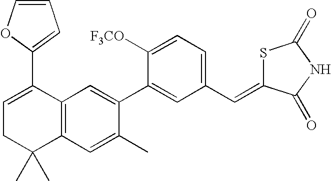

Prepared in a similar manner to example 1 using 4-Trifluoromethoxy-3-(3,5,5-trimethyl-8-thiophen-2-yl-5,6-dihydro-naphthalen-2-yl)-benzaldehyde. mp 146° C. ¹H NMR (300 MHz, DMSO-d-6): 1.26 (s, 6H), 2.08 (s, 3H), 2.23 (m, 2H), 5.94 (t, J=4.5 Hz, 1H), 5.97 (t, J=3.8 Hz, 1H), 6.48 (d, J=9.7 Hz, 2H), 6.90 (s, 1H), 7.27 (s, 1H), 7.61 (s, 1H), 7.63 (d, J=8.8 Hz, 1H), 7.73 (d, J=8.8 Hz, 1H), 7.86 (s, 1H), 12.70 (br s, 1H).

The intermediate 4-Trifluoromethoxy-3-(3,5,5-trimethyl-8-thiophen-2-yl-5,6-dihydro-naphthalen-2-yl)-benzaldehyde was prepared in a similar manner to example 5a using trifluoro-methanesulfonic acid 7-(5-[1,3]dioxolan-2-yl-2-trifluoromethoxy-phenyl)-4,4,6-trimethyl-3,4-dihydro-naphthalen-1-yl ester (example 5b) and 2-furanboronic acid.

### Example 8

### 5-[2,5-Difluoro-4-methoxy-3-(3,5,5,8,8-pentamethyl-5,8-dihydro-naphthalen-2-yl)-benzylidene]-thiazolidine-2,4-dione, which can be referred to as “Compound 8”

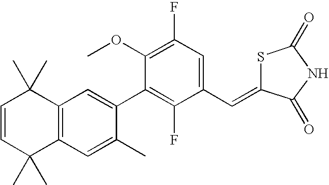

Prepared in a similar manner to example 1 using 2,5-Difluoro-4-methoxy-3-(3,5,5,8,8-pentamethyl-5,8-dihydro-naphthalen-2-yl)-benzaldehyde. mp 244° C. ¹H NMR (300 MHz; DMSO) 1.25 (s, 6H), 1.31 (s, 6H), 2.03 (s, 3H), 3.74 (s, 3H), 5.51 (s, 2H), 7.22 (s, 1H), 7.34 (s, 1H), 7.41 (m, 1H), 7.68 (s, 1H), 12.73 (br s, 1H).

The intermediate 2,5-Difluoro-4-methoxy-3-(3,5,5,8,8-pentamethyl-5,8-dihydro-naphthalen-2-yl)-benzaldehyde was prepared as followed:

a) 2,5-Difluoro-4-methoxy-3-(3,5,5,8,8-pentamethyl-5,8-dihydro-naphthalen-2-yl)-benzaldehyde:

A solution of 3,5,5,8,8-pentamethyl-5,8-dihydro-naphthalen-2-boronic acid (1.067 g, 4.37 mmol), 3-bromo-2,5-difluoro-4-methoxy benzaldehyde (example 3c) (987 mg, 3.93 mmol) and potassium carbonate (1.08 g, 7.87 mmol) in a mixture of toluene (14 mL), ethanol (2.5 mL) and water (1.5 mL) was degassed with argon for 30 minutes. Pd(PPh₃)₄ (136 mg, 0.12 mmol) was added and the mixture was refluxed for 15 hours. The reaction was cooled to room temperature, and extracted with ethyl acetate, washed with water and brine, dried over MgSO₄, filtered and evaporated. The residue was chromatographed on silica gel (5% ethylacetate in hexane) to give 0.714 g of 2,5-Difluoro-4-methoxy-3-(3,5,5,8,8-pentamethyl-5,8-dihydro-naphthalen-2-yl)-benzaldehyde (49%). ¹H NMR (300 MHz; CDCl₃) 1.32 (s, 6H), 1.38 (s, 6H), 2.14 (s, 3H), 3.83 (d, 3H), 5.53 (s, 2H), 7.12 (s, 1H), 7.64 (m, 1H), 10.26 (d, 1H).

b) 3,5,5,8,8-pentamethyl-5,8-dihydro-naphthalen-2-boronic acid.

To a mixture of 6-bromo-1,1,4,4,7-pentamethyl-1,4-dihydro-naphthalen (3.16 g, 11.31 mmol) in THF (15 mL) cooled to −78° C. under an atmosphere of argon was added n-BuLi (12.7 mL, 1.6 M, 20.36 mmol) dropwise. The resulting suspension was stirred for 5 minutes and triisopropylborate (7.8 mL, 39.94 mmol) was added dropwise via syringe. The mixture was stirred at −50° C. for 2 hours then warmed up to room temperature and stirred overnight at room temperature. 1.0 N HCl (20 mL) was slowly added to the reaction mixture. After 30 minutes the mixture was diluted with ethyl acetate and the layers separated, the aqueous layer was extracted once with ethyl acetate and the two organic layers combined. The resulting organic layer was washed with water, brine and dried (MgSO₄), filtered and evaporated. The residue was chromatographed on silica gel (20% ethyl acetate in hexane) to give 1.975 g of 3,5,5,8,8-pentamethyl-5,8-dihydro-naphthalen-2-boronic acid (72%). ¹H NMR (300 MHz; CDCl₃) 1.38 (s, 6H), 1.40 (s, 6H), 2.85 (s, 3H), 5.53 (2s, 2H), 7.27 (s, 1H), 8.35 (s, 1H).

c) 6-bromo-1,1,4,4,7-pentamethyl-1,4-dihydro-naphthalen.

A suspension of 6-bromo-1,1,4,4,7-pentamethyl-3,4-dihydro-1H-naphthalen-2-one (1.044 g, 3.54 mmol), p-toluenesulfonylhydrazine (790 mg, 4.24 mmol) and p-toluenesulfonic acid (134 mg, 0.71 mmol) in methanol (20 mL) was heated at reflux under argon for 16 hours. Some of the solvent was removed under reduced pressure. The compound crystallised and was collected to give 1.114 g of the hydrazide intermediate. The later was was dissolved in t-butyl methyl ether (20 mL) and treated with MeLi. LiBr (1.5 M in Et₂O, 4.8 mL, 3 eq) at room temperature under argon. After 1 hour the reaction was quenched with water, extracted with ether. The organic was dried (MgSO₄), filtered and evaporated to give 674 mg of 6-bromo-1,1,4,4,7-pentamethyl-1,4-dihydro-naphthalen. ¹H NMR (300 MHz; CDCl₃) 1.31 (s, 12H), 2.37 (s, 3H), 5.48 (2s, 2H), 7.20 (s, 1H), 7.48 (s, 1H).

d) 6-bromo-1,1,4,4,7-pentamethyl-3,4-dihydro-1H-naphthalen-2-one.

A solution of 1,1,4,4,7-pentamethyl-3,4-dihydro-1H-naphthalen-2-one (1.139 g, 5.26 mmol) in dichloromethane (10 mL) at 0° C. was treated with AlCl₃ (1.4 g, 10.53 mmol) and stirred for 5 minutes. A solution of bromine (0.28 mL, 5.53 mmol) was added dropwise and the reaction stirred at 0° C. for 30 minutes. The reaction was then poured into ice and extracted with ethyl acetate. The organic phase was further washed with water, brine and dried (MgSO₄), filtered and evaporated. The residue was triturated wit methanol and collected to give 1.047 g of 6-bromo-1,1,4,4,7-pentamethyl-3,4-dihydro-1H-naphthalen-2-one (67%). ¹H NMR (300 MHz; CDCl₃) 1.28 (s, 6H), 1.42 (s, 6H), 2.39 (s, 3H), 2.60 (s, 2H), 7.15 (s, 1H), 7.50 (s, 1H).

e) 1,1,4,4,7-pentamethyl-3,4-dihydro-1H-naphthalen-2-one.

To a stirred mixture of dihydro-2,2,5,5-tetramethyl-3(2H)-furanone (20 mL, 0.13 mol) in toluene (150 mL) was added gradually AlCl₃ (34.73 g, 0.26 mol) while maintening the mixture between 40° C. and 50° C. The solution was the heated at 70° C. for 20 minutes. The solution was cooled to 0° C. and 1N HCl (150 mL) was added slowly. The layers were separated. The aqueous layer was extracted with ethyl acetate and the organic phase was further washed with water, saturated aqueous NaHCO3, water, dried (MgSO₄), filtered and evaporated. The residue was chromatographed on silica gel (3% ethyl acetate in hexane) to give 7.63 g of 1,1,4,4,7-pentamethyl-3,4-dihydro-1H-naphthalen-2-one (27%). ¹H NMR (300 MHz; CDCl₃) 1.31 (s, 6H), 1.45 (s, 6H), 2.35 (s, 3H), 2.63 (s, 2H), 7.10 (m, 2H), 7.26 (d, J=8, 1 Hz, 1H).

### Example 9

### 5-[3-(3,5,5,8,8-Pentamethyl-5,8-dihydro-naphthalen-2-yl)-4-trifluoromethoxy-benzylidene]-thiazolidine-2,4-dione, which can be referred to as “Compound 9”

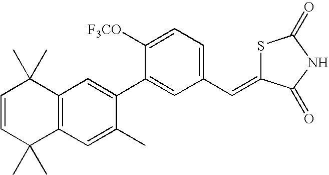

Prepared in a similar manner to example 1 using 3-(3,5,5,8,8-Pentamethyl-5,8-dihydro-naphthalen-2-yl)-4-trifluoromethoxy-benzaldehyde. mp 204° C. ¹H NMR (300 MHz; DMSO) 1.27 (s, 6H), 1.34 (s, 6H), 2.09 (s, 3H), 5.55 (s, 2H), 7.19 (s, 1H), 7.37 (s, 1H), 7.65 (m, 2H), 7.73 (dd, J₁=8.8 Hz, J₂=2.1 Hz, 1H), 7.88 (s, 1H), 12.70 (br s, 1H).

The intermediate 3-(3,5,5,8,8-Pentamethyl-5,8-dihydro-naphthalen-2-yl)-4-trifluoromethoxy-benzaldehyde was prepared in a similar manner to example 8a using 3,5,5,8,8-pentamethyl-5,8-dihydro-naphthalen-2-boronic acid (example 8b) and 3-bromo-4-trifluoromethoxy benzaldehyde (example 1j). ¹H NMR (300 MHz; CDCl₃) 1.32 (s, 6H), 1.38 (s, 6H), 2.11 (s, 3H), 5.53 (s, 2H), 7.13 (s, 1H), 7.24 (s, 1H), 7.33 (d, J=1.2 Hz, 1H), 7.49 (dd, J₁=1.8 Hz, J₂=8.4 Hz, 1H), 7.86 (d, J=2.1 Hz, 1H), 7.94 (dd, J₁=2.1 Hz, J₂=8.7 Hz, 1H), 10.03 (s, 1H).

### Example 10

### 5-[4-Dimethylamino-3-(3,5,5,8,8-pentamethyl-5,8-dihydro-naphthalen-2-yl)-benzylidene]-thiazolidine-2,4-dione, which can be referred to as “Compound 10”

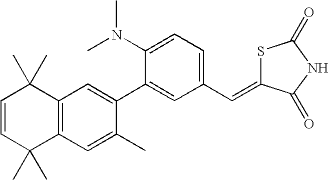

Prepared in a similar manner to example 1 using 4-Dimethylamino-3-(3,5,5,8,8-pentamethyl-5,8-dihydro-naphthalen-2-yl)-benzaldehyde. mp 232° C. ¹H NMR (300 MHz; DMSO): 1.27 (s, 3H), 1.30 (s, 3H), 1.36 (s, 6H), 2.09 (s, 3H), 2.60 (s, 6H), 5.55 (s, 2H), 5.85 (br s, 1H), 7.14 (br s, 1H), 7.21-7.35 (m, 3H), 7.50 (d, J=8.5 Hz, 1H), 7.75 (s, 1H), 12.47 (br s, 1H).

The intermediate 4-Dimethylamino-3-(3,5,5,8,8-pentamethyl-5,8-dihydro-naphthalen-2-yl)-benzaldehyde was prepared in a similar manner to example 8a using 3,5,5,8,8-pentamethyl-5,8-dihydro-naphthalen-2-boronic acid (example 8b) and 3-bromo-4-dimethylamino benzaldehyde (example 2a). ¹H NMR (300 MHz; CDCl₃) 1.29 (s, 3H), 1.31 (s, 3H), 1.36 (s, 3H), 1.37 (s, 3H), 2.11 (s, 3H), 2.66 (s, 6H), 5.53 (s, 2H), 6.94 (d, J=8.4 Hz, 1H), 7.20 (s, 1H), 7.33 (br s, 1H), 7.61 (d, J=2.1 Hz, 1H), 7.75 (dd, J₁=2.1 Hz, J₂=8.7 Hz, 1H), 9.81 (s, 1H).

### Example 11

### 5-[2,5-Difluoro-4-methoxy-3-(1,1,4,4,7-pentamethyl-isochroman-6-yl)-benzylidene]-thiazolidine-2,4-dione, which can be referred to as “Compound 11”

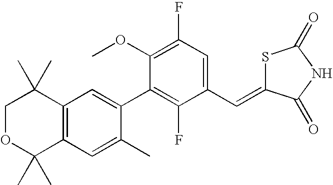

Prepared in a similar manner to example 1 using 2,5-Difluoro-4-methoxy-3-(1,1,4,4,7-pentamethyl-isochroman-6-yl)-benzaldehyde. mp 142° C. ¹H NMR (300 MHz; DMSO): 1.18 (s, 6H), 1.49 (s, 6H), 2.03 (s, 3H), 3.52 (s, 2H), 3.77 (d, J=2.05 Hz, 3H), 7.15 (s, 1H), 7.17 (s, 1H), 7.43 (dd, J₁=6.9 Hz, J₂=12 Hz, 1H), 7.70 (s, 1H), 12.76 (s, 1H).

The intermediate 2,5-Difluoro-4-methoxy-3-(1,1,4,4,7-pentamethyl-isochroman-6-yl)-benzaldehyde was prepared as followed:

a) 2,5-Difluoro-4-methoxy-3-(1,1,4,4,7-pentamethyl-isochroman-6-yl)-benzaldehyde:

Prepared in a similar manner to example 1a using 1,1,4,4,7-Pentamethyl-isochroman-6-boronic acid and 3-bromo-2,5-difluoromethoxy benzaldehyde (example 3c). ¹H NMR (300 MHz; CDCl₃) 1.25 (s, 6H), 1.58 (s, 6H), 2.12 (s, 3H), 3.61 (s, 2H), 3.85 (d, J=3.0 Hz, 3H), 6.99 (s, 1H), 7.06 (s, 1H), 7.61 (dd, J₁=6.6 Hz, J₂=11.4 Hz, 1H), 10.26 (d, J=0.6 Hz, 1H).

b) 1,1,4,4,7-Pentamethyl-isochroman-6-boronic acid:

Prepared in a similar manner to example 1b using 6-bromo-1,1,4,4,7-pentamethyl-isochroman. ¹H NMR (300 MHz; CDCl₃) 1.31 (s, 6H), 1.57 (s, 6H), 2.81 (s, 3H), 3.63 (s, 2H), 6.98 (s, 1H), 8.24 (s, 1H).
c) 6-bromo-1,1,4,4,7-pentamethyl-isochroman.

To a solution of 1,1,4,4,7-pentamethyl-isochroman (9.13 g, 44.69 mmol) in nitromethane (30 mL), cooled to 0° C., was added under argon AlCl₃ (1.2 g, 8.94 mmol) followed by Br₂ (2.4 mL, 46.92 mmol) slowly. The reaction mixture was stirred for 5 hrs at room temperature. The solution was fitered over celite, and evaporated to give 12.15 g of 6-bromo-1,1,4,4,7-pentamethyl-isochroman (96%). ¹H NMR (300 MHz; CDCl₃) 1.24 (s, 6H), 1.50 (s, 6H), 2.34 (s, 3H), 3.55 (s, 2H), 6.91 (s, 1H), 7.42 (s, 1H).

d) 1,1,4,4,7-pentamethyl-isochroman.

To a solution trifluoro-methanesulfonic acid-1,1,4,4,7-pentamethyl-isochroman-6-yl ester (16.5 g, 46.83 mmol) in dry DMF (90 mL) was added under argon Pd(OAc)₂ (420 mg, 1.87 mmol), 1,1′-bis(diphenylphosphino)ferrocene (1.04 g, 1.87 mmol), Et₃N (19.6 mL, 140.50 mmol) and formic acid (3.6 mL, 93.66 mmol). The reaction mixture was heated at 60° C. for 4 hours then cooled to room temperature. Cold water (60 mL) wad added slowly and the solution extracted with ether. The organic phase was further washed with water and brine, dried over magnesium sulfate, filtered and evaporated. The residue was chromatographed on silica gel (5 to 10% ethylacetate in hexane) to give 9.14 g of 1,1,4,4,7-pentamethyl-isochroman (96%). ¹H NMR (300 MHz; CDCl₃) 1.25 (s, 6H), 1.52 (s, 6H), 2.31 (s, 3H), 3.57 (s, 2H), 6.87 (s, 1H), 7.02 (d, J=8.1 Hz, 1H), 7.19 (d, J=8.1 Hz, 1H).

e) Trifluoro-methanesulfonic acid-1,1,4,4,7-pentamethyl-isochroman-6-yl ester.

To a solution 1,1,4,4,7-pentamethyl-isochroman-6-ol (11.3 g, 51.29 mmol) in dry dichloromethane (150 mL) was added slowly under argon pyridine (5 mL, 61.55 mmol). The solution was cooled to 0° C., then triflic anhydride (10.4 mL, 61.55 mmol) was added dropwise. The reaction mixture was warmed to room temperature slowly and stirred overnight at room temperature. The solution was washed successively with water, 1N HCl, water, satuared aqueous NaHCO₃ water and brine, dried over magnesium sulfate, filtered and evaporated to give 17.6 g of trifluoro-methanesulfonic acide-1,1,4,4,7-pentamethyl-isochroman-6-yl ester (97%). ¹H NMR (300 MHz; CDCl₃) 1.25 (s, 6H), 1.52 (s, 6H), 2.32 (s, 3H), 3.57 (s, 2H), 6.96 (s, 1H), 7.10 (s, 1H).

f) 1,1,4,4,7-pentamethyl-isochroman-6-ol.

To a solution 7-Diethylaminomethyl-1,1,4,4,7-tetramethyl-isochroman-6-ol. (20.28 g, 69.59 mmol) in dry ether (30 mL) was added slowly under argon dimethyl sulfate (13.2 mL, 139.18 mmol). The reaction mixture was stirred at room temperature overnight. The solid was collected (24.76 g) and dissolved in methanol (55 mL) and water (1 mL). Palladium hydroxyde (20 wt % Pd on carbon, 8.4 g, 0.1 eq) was added and the reaction hydrogenated for 48 hours (60 Psi). The solution was filtered over celite and evaporated. The residue was dissolved in ethylacetate and further washed with water, 2N HCl, water, sat. NaHCO3, water and brine to give 11.3 g of 1,1,4,4,7-pentamethyl-isochroman-6-ol (86.5%). ¹H NMR (300 MHz; CDCl₃) 1.22 (s, 6H), 1.50 (s, 6H), 2.21 (s, 3H), 3.57 (s, 2H), 4.77 (s, 1H), 6.70 (s, 1H), 6.80 (s, 1H).

g) 7-Diethylaminomethyl-1,1,4,4,7-tetramethyl-isochroman-6-ol.

To a solution of 1,1,4,4-tetramethyl-isochroman-6-ol (15 g, 68.10 mmol) in ethanol (25 mL) was added slowly formaldehyde (7.6 mL, 102.15 mmol) and diethylamine (10.6 mL, 102.15 mmol) followed by water (6.25 mL0. The reaction mixture was refluxed for 4 hours. Ethanol was removed under reduced pressure and the solution extracted with ethylacetate. The organic phase was further washed with water and brine, dried over magnesium sulfate, filtered and evaporated to give 20.3 g of 7-Diethylaminomethyl-1,1,4,4,7-tetramethyl-isochroman-6-ol (97.6%). ¹H NMR (300 MHz; CDCl₃) 1.09 (t, J=6.9 Hz, 3H), 1.23 (s, 6H), 1.47 (s, 6H), 2.62 (q, J=7.5 Hz, 2H), 3.56 (s, 2H), 3.71 (s, 2H), 6.62 (s, 1H), 6.70 (s, 1H).

h) 1,1,4,4-tetramethyl-isochroman-6-ol.

A solution of 3-(2-hydroxy-1,1-dimethyl-ethyl)-phenol (30 g, 0.18 mol) in acetone (93 mL) was cooled to 0° C. and concentrated hydrochloric acid (25 mL) was added dropwise. The reaction mixture was warmed up slowly to room temperature and stirred overnight then poured into ice/water (150 mL) the extracted with ether. The organic layer was washed with water and brine, dried over magnesium sulfate, filtered and evaporated to give 34.06 g of 1,1,4,4-tetramethyl-isochroman-6-ol (92%). ¹H NMR (300 MHz; CDCl₃) 1.24 (s, 6H), 1.51 (s, 6H), 3.58 (s, 2H), 5.51 (br s, 1H), 6.64 (dd, J₁=2.4 Hz, J₂=8.4 Hz, 1H), 6.77 (d, J=3 Hz, 1H), 6.95 (d, J=8.4 Hz, 1H).

i) 3-(2-hydroxy-1,1-dimethyl-ethyl)-phenol.

To a solution of lithium aluminum hydride (1M in THF, 258 mL, 0.258 mol) colled to 0° C. was added under argon dropwise a solution of 2-(3-hydroxy-phenyl)-2-methyl-propionic acid ethyl ester (42.7 g, 0.205 mol) in dry THF (100 mL). After the addition was completed the reaction mixture was warmed to room temperature slowly and stiired for 3 hours. The solution was then cooled to 0° C. and ice/water was added very slowly to quench the excess of reagent, then 2N HCl was added to pH=6. The solution was extracted with ethylacetate and washed with water and brine, dried over NaSO₄, filtered and evaporated to give 37.9 g of 3-(2-hydroxy-1,1-dimethyl-ethyl)-phenol. ¹H NMR (300 MHz; CDCl₃) 1.30 (s, 6H), 3.59 (s, 2H), 5.40 (br s, 1H), 6.77 (dd, J₁=2.7 Hz, J₂=8.1 Hz, 1H), 6.85 (t, J=2.1 Hz, 1H), 6.93 (d, J=8.1 Hz, 1H), 7.20 (t, J=8.1 Hz, 1H).

j) 2-(3-hydroxy-phenyl)-2-methyl-propionic acid ethyl ester.

A solution of 2-(3-methoxy-phenyl)-2-methyl-propionic acid ethyl ester (59.2 g, 0.266 mol) in dry dichloromethane (250 mL) was cooled to −78° C. under argon, and boron tribromide (27.7 mL, 0.293 mmol) was added slowly. The reaction mixture was then warmed up slowly to room temperature and stirred ovgernight. The solution was treated at 0° C. with saturated aqueous NaHCO₃ to pH=7-8 and extracted with dichloromethane. The organic phase was washed with water and brine, dried (MgSO₄), filtered and evaporated to give 42.7 g of 2-(3-hydroxy-phenyl)-2-methyl-propionic acid ethyl ester (77%). ¹H NMR (300 MHz; CDCl₃) 1.18 (t, J=6.9 Hz, 3H), 1.55 (s, 6H), 4.13 (q, J=6.9 Hz, 2H), 6.72 (dd, J₁=2.1 Hz, J₂=7.8 Hz, 1H), 6.83 (t, J=2.1 Hz, 1H), 6.88 (d, J=8.1 Hz, 1H), 7.18 (t, J=8.1 Hz, 1H).

k) 2-(3-methoxy-phenyl)-2-methyl-propionic acid ethyl ester.

To a cooled (−78° C.) solution of lithium diisopropylamine (2M in THF/n-heptane, 288 mL, 0.576 mol) was added under argon a solution of 2-(3-methoxy-phenyl)-propionic acid ethyl ester (60 g, 0.288 mol) in dry THF (300 mL). The reaction mixture was stirred at −78° C. for 1 hour then iodomethane (40 mL, 0.634 mol) was added dropwise and the reaction mixture allowed to warmed up slowly to room temperature and stirred overnight. The recation mixture was poured into ice-water and 2N HCl was added to pH=7. The layers were separated, the aqueous phase was extracted with ether, the organic combined and further washed with water and brine, dried (MgSO₄), filtered and evaporated. Distillation (90° C., high vaccum) provided 59.2 g of 2-(3-methoxy-phenyl)-2-methyl-propionic acid ethyl ester (92%). ¹H NMR (300 MHz; CDCl₃) 1.19 (t, J=7.2 Hz, 3H), 1.56 (s, 6H), 3.80 (s, 1H), 4.12 (q, J=7.2 Hz, 2H), 6.77 (dd, J₁=2.7 Hz, J₂=8.4 Hz, 1H), 6.89 (t, J=2.1 Hz, 1H), 6.90 (d, J=8.1 Hz, 1H), 7.25 (t, J=7.5 Hz, 1H).

l) 2-(3-methoxy-phenyl)-propionic acid ethyl ester.

Prepared in a similar maner as described in example 11k using 3-methoxy-phenyl-acetic acid ethyl ester. ¹H NMR (300 MHz; CDCl₃) 1.21 (t, 3H), 1.49 (d, 3H), 3.66 (m, 1H), 3.80 (s, 1H), 4.12 (q, 2H), 6.94 (m, 2H), 7.23 (m, 2H).

### Example 12

### 5-[2,5-Difluoro-4-methoxy-3-(1,1,4,4,7-pentamethyl-isochroman-6-yl)-benzylidene]-thiazolidine-2,4-dione, Tris salt, which can be referred to as “Compound 12”

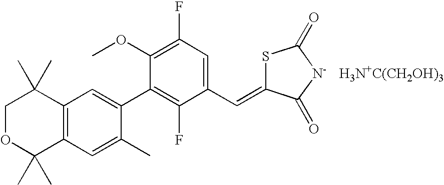

Compound 11 (6.87 g, 14.51 mmol) was dissolved in dry THF (50 mL) and a solution of tris(hydroxymethyl)aminomethane (“Tris,” 1.76 g, 14.51 mmol) in dry methanol (50 mL) was added dropwise at room temperature. The reaction mixture was stirred 48 hrs at room temperature, filtered and evaporated. The residue was redissolved in ethanol, evaporated and dried under high vacuum to afford 8.6 g of: 5-[2,5-Difluoro-4-methoxy-3-(1,1,4,4,7-pentamethyl-isochroman-6-yl)-benzylidene]-thiazolidine-2,4-dione.TRIS. ¹H-NMR (300 MHz, DMSO-d-6): 1.19 (s, 6H), 1.50 (s, 6H), 2.03 (s, 3H), 3.47 (s, 6H), 3.52 (s, 2H), 3.71 (d, J=2.1 Hz, 3H), 5.17 (s, 3H), 7.14 (s, 1H), 7.17 (s, 1H), 7.34 (s, 1H), 7.42 (dd, J₁=7.5 Hz, J₂=12.6 Hz, 1H), 7.58 (br s, 2H).

### Example 13

### 5-[3-(1,1,4,4,7-Pentamethyl-isochroman-6-yl)-4-trifluoromethoxy-benzylidene]-thiazolidine-2,4-dione, which can be referred to as “Compound 13”

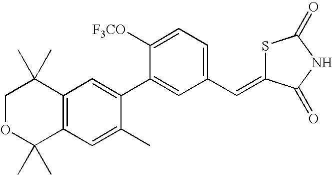

Prepared in a similar manner to example 1 using 3-(1,1,4,4,7-Pentamethyl-isochroman-6-yl)-4-trifluoromethoxy-benzaldehyde. mp 255° C. ¹H NMR (300 MHz; DMSO): 1.17 (s, 6H), 1.49 (s, 6H), 2.06 (s, 3H), 3.51 (s, 2H), 7.11 (s, 1H), 7.15 (s, 1H), 7.62-7.73 (m, 3H), 7.85 (s, 1H), 12.8 (s, 1H).

The intermediate 3-(1,1,4,4,7-Pentamethyl-isochroman-6-yl)-4-trifluoromethoxy-benzaldehyde was prepared as followed:

a) 3-(1,1,4,4,7-Pentamethyl-isochroman-6-yl)-4-trifluoromethoxy-benzaldehyde.

Prepared in a similar manner to example 1a using 1,1,4,4,7-Pentamethyl-6-(4,4,5,5-tetramethyl-[1,3,2]dioxoborolan-2-yl)-isochroman and 3-bromo-4-trifluoromethoxy benzaldehyde (example 1j). ¹H NMR (300 MHz; CDCl₃) 1.25 (s, 6H), 1.57 (s, 6H), 2.08 (s, 3H), 3.61 (s, 2H), 6.96 (s, 1H), 7.07 (s, 1H), 7.49 (dd, J₁=1.5 Hz, J₂=8.4 Hz, 1H), 7.85 (d, J=1.8 Hz, 1H), 7.95 (dd, J=1.8 Hz, J₂=8.4 Hz, 1H), 10.04 (s, 1H).

b) 1,1,4,4,7-Pentamethyl-6-(4,4,5,5-tetramethyl-[1,3,2]dioxoborolan-2-yl)-isochroman.

A solution of trifluoro-methanesulfonic acid-1,1,4,4,7-pentamethyl-isochroman-6-yl ester (example 1e) (620 mg, 1.76 mmol), bis(pinacolato)diboran (492 mg, 1.94 mmol), PdCl₂(dppf) (43 mg, 0.053 mmol), dppf (30 mg, 0.053 mmol), KOAc (518 mg, 5.28 mmol) in dioxane (10 mL) was heated at 80° C. for 4 hours under argon. Water was added and the solution extracted with ethyl acetate. The organic was washed with water and brine, dried over magnesium sulfate, filtered and evaporated. The residue was chromatographed on silca gel (20% ethylacetate in hexane) to give 560 mg of ,1,4,4,7-Pentamethyl-6-(4,4,5,5-tetramethyl-[1,3,2]dioxoborolan-2-yl)-isochroman (96%).). ¹H NMR (300 MHz; CDCl₃) 1.28 (s, 6H), 1.32 (s, 12H), 1.57 (s, 6H), 2.49 (s, 2H), 3.56 (s, 2H), 6.86 (s, 1H), 7.70 (s, 1H).

### Example 14

### 5-[4-Dimethylamino-3-(1,1,4,4,7-pentamethyl-isochroman-6-yl)-benzylidene]-thiazolidine-2,4-dione, which can be referred to as “Compound 14”

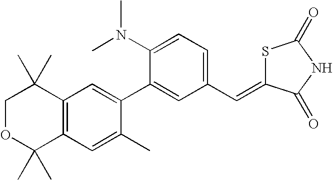

Prepared in a similar manner to example 1 using 4-Dimethylamino-3-(1,1,4,4,7-pentamethyl-isochroman-6-yl)-benzaldehyde. mp 269° C. ¹H NMR (300 MHz; DMSO): 1.16 (s, 3H), 1.18 (s, 3H), 1.46 (s, 6H), 2.04 (s, 3H), 2.53 (s, 6H), 3.49 (d, J=2.34 Hz, 2H), 7.05 (d, J=8.50 Hz, 1H), 7.06 (s, 1H), 7.13 (s, 1H), 7.21(d, J=2.05 Hz, 1H), 7.45 (dd, 1H, J₁=8.50, J₂=2.05 Hz), 7.71 (s, 1H).

The intermediate 4-Dimethylamino-3-(1,1,4,4,7-pentamethyl-isochroman-6-yl)-benzaldehyde was prepared in a similar manner to example 1a using trifluoro-methanesulfonic acid-1,1,4,4,7-pentamethyl-isochroman-6-yl ester (example 1e) and 6-dimethylamino-3-formyl-1-phenyl boronic acid. ¹H NMR (300 MHz; CDCl₃) 1.23 (s, 3H), 1.27 (s, 3H), 1.55 (s, 3H), 1.56 (s, 3H), 2.08 (s, 3H), 2.65 (s, 6H), 3.60 (d, J=4.5 Hz, 1H), 6.90 (s, 1H), 6.94 (d, J=8.4 Hz, 1H), 7.16 (s, 1H), 7.57 (s, 1H), 7.74 (d, J=7.8 Hz, 1H), 9.81 (s, 1H).

a) 6-dimethylamino-3-formyl-1-phenyl boronic acid.

To a mixture of 2-(3-bromo-4-dimethylamino-1-phenyl)-1,3-dioxolane (8.8 g, 32.34 mmol) in THF (80 mL) cooled to −78° C. under an atmosphere of argon was added n-BuLi (19.4 mL, 2.5 M, 48.50 mmol) dropwise. The resulting suspension was stirred for 5 minutes and triisopropylborate (22.4 mL, 97.0 mmol) was added dropwise via syringe. The mixture was stirred at −50° C. for 2 hours then warmed up to room temperature and stirred overnight at room temperature. 1.0 N HCl (50 mL) was slowly added to the reaction mixture. After 4 hours 10% aqueous potassium carbonate was added to the reaction mixture until pH=6˜7. The solution was diluted with ethyl acetate and the layers separated. The organic layer was further washed with water, brine and dried (MgSO₄). The mixture was filtered and evaporated to afford 6.4 g of crude 6-dimethylamino-3-formyl-1-phenyl boronic acid used without further purification in the Suzuki coupling.

b) 2-(3-bromo-4-dimethylamino-1-phenyl)-1,3-dioxolane.

To a solution of 3-bromo-4-dimethylamino-benzaldehyde (example 2a) (10 g, 43.84 mmol) in toluene (80 mL) was added ethylene glycol (48.9 mL, 877 mmol) and p-toluenesulfonic acid monohydrate (0.5 g, 2.63 mmol). The reaction mixture was heated at reflux overnight and the water was removed using a Dean Stark apparatus. The solution was cooled to room temperature, aqueous potassium carbonate (10%) was added and the solution extracted with ethyl acetate. The organic layer was washed with water, brine and dried (MgSO₄). The residue was purified on silica gel (eluent: 10% ethyl acetate in hexane) to give 10.84 g of 2-(3-bromo-4-dimethylamino-1-phenyl)-1,3-dioxolane. (90%). ¹H NMR (300 MHz; CDCl₃): δ 2.81 (s, 6H), 4.02 (m, 2H), 4.13 (m, 2H), 5.74 (s, 1H), 7.06 (d, J=8.1 Hz, 1H), 7.43 (dd, J=1.1 Hz and 8.4 Hz, 1H), 7.69 (d, J=1.5 Hz, 1H).

### Example 15

### 5-[3-(7-Chloro-1,1,4,4-tetramethyl-isochroman-6-yl)-4-trifluoromethoxy-benzylidene]-thiazolidine-2,4-dione, which can be referred to as “Compound 15”

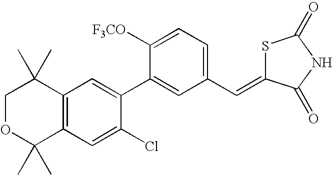

Prepared in a similar manner to example 1 using 3-(7-Chloro-1,1,4,4-tetramethyl-isochroman-6-yl)-4-trifluoromethoxy-benzaldehyde. mp 255° C. ¹H NMR (300 MHz; DMSO): 1.20 (s, 6H), 1.51 (s, 6H), 3.54 (s, 2H), 7.38 (s, 1H), 7.48 (s, 1H), 7.66 (m, 1H), 7.70 (d, J=2.3 Hz, 1H), 7.77 (dd, J₁=2.3 Hz, J₂=8.5 Hz, 1H), 7.87 (s, 1H), 12.72 (bs, 1H).

The intermediate 3-(7-Chloro-1,1,4,4-tetramethyl-isochroman-6-yl)-4-trifluoromethoxy-benzaldehyde was prepared in a similar manner to example 1a using trifluoro-methanesulfonic acid 7-chloro-1,1,4,4-tetramethyl-isochroman-6-ester and 3-formy-6-trifluoromethoxy-1-phenyl boronic acid (example 5e). ¹H NMR (300 MHz; CDCl₃) 1.26 (s, 6H), 1.57 (s, 6H), 3.61 (s, 2H), 7.20 (d, J=3.6 Hz, 1H), 7.51 (dd, Jhd 1 =1.2 Hz, J₂=8.1 Hz, 1H), 7.90 (d, J=2.1 Hz, 1H), 7.99 (m, 1H), 10.04 (s, 1H).

a) Trifluoro-methanesulfonic acid 7-chloro-1,1,4,4-tetramethyl-isochroman.

Prepared in a similar manner to example 11e using 7-chloro-1,1,4,4-tetramethyl-isochroman-6-ol. ¹H NMR (300 MHz; CDCl₃) 1.26 (s, 6H), 1.53 (s, 6H), 3.58 (s, 2H), 7.19 (s, 1H), 7.26 (s, 1H).

b) 7-Chloro-1,1,4,4-tetramethyl-isochroman-6-ol.

To a solution of 1,1,4,4-tetramethyl-isochroman-6-ol (example 11h) (4.18 g, 20.3 mmol) in dichlorometane (20 mL) was added at 0° C. under argon a solution of thionyl chloride (1M in dichloromethane, 22.3 mL, 1.1 eq). The reaction mixture was stirred at 0° C. for 4 hours then water was added and the layers separated. The organic phase was further washed with water and brine, dried over magnesium sulfate, filtered and evaporated. The residue was chromatographed on silica gel (15% ethyl acetate in hexane) to give 3.27 g of 7-Chloro-1,1,4,4-tetramethyl-isochroman-6-ol (62%).

### Example 16

### Substitued-(8-isopropyl-3,5,5-trimethyl-5,6-dihydro-naphthalen-2-yl)-benzylidene]-thiazolidine-2,4-dione library Procedure

The compounds recited in the examples above were synthesized in a traditional manner and not as a mixture of compounds. The building blocks used in the synthesis of a library of compounds were prepared using procedures similar to those described above, or, similar libraries of related substituted heterocyclic compounds may be produced by these or other alternative chemical reaction steps known by one skilled in the art. In the current example, the boronic acids and benzaldehyde bromides/iodides are shown below along with their respective codes that were employed during the synthesis of a combinatorial library of the compounds of the invention:

Boronic Acid:

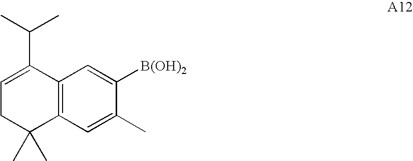

Benzaldehydes:

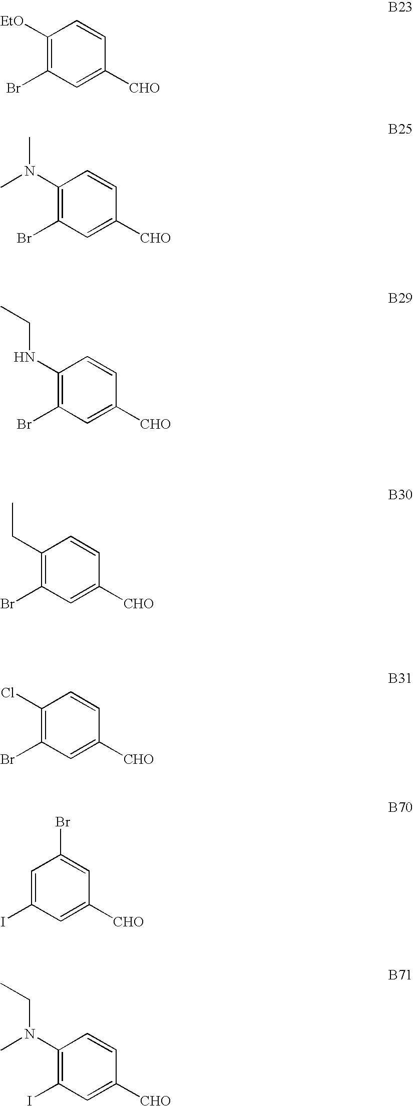

Heterocycles:

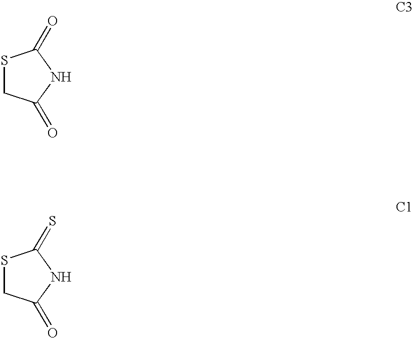

1) Suzuki Coupling Step

A solution of 0.225 mmol of a boronic acid, (0.150 mmol) of aldehyde, and 62.2 mg (0.450 mmol) of K₂CO₃ in 1.0 mL of toluene and 0.5 mL of ethanol-water (1.2:1) was degassed with argon in a glove bag for three times and was then treated with a solution of 11.8 mg (0.010 mmol) of Tetrakis(triphenylphosphine)-palladium (0) in 0.5 mL of toluene at RT. The reaction was then heated at 85° C., with vigorous shaking or stirring, for a period of 16 hr under argon atmosphere. The reaction was cooled down to RT, dried over anhydrous Na₂SO₄, and purified by a short silica gel column (1 cm diameter and 3 cm length). The column was eluted with 2 mL of toluene and 3 mL of 60% EtOAc in hexane sequentially. The combined eluents were concentrated under reduced pressure to give relative pure desired product which was used in the next step directly.

2) Knoevenagel Condensation Step

The coupling product from the Suzuski step was dissolved in 1.5 mL of toluene and approximately 0.5 mL was added to a reaction was vial. The reaction vial was treated with 6 mg (0.045 mmol) of rhodanine (C1) or 5.3 mg (0.045 mmol) of thiazolidine-2,4-dione (C2), and 0.005 mmol of piperidinium acetate or 0.15 mmol of ammonium acetate. The resulting reaction mixture was heated at 80° C. for 3 hr and cooled to room temperature to form a suspension. The solids that precipitated upon cooling were typically desired products with very high purity. The oily products with relative low purity could be further purified by chromatography.

3) Quality Control of the Library

Mass spectra analysis conditions used in QC:
Flow Injection Analysis (FIA) mass spectrometry
Period: 1 minute
Ionization: pneumatically (N₂) assisted electrospray
Polarity: negative
Mobile phase: methanol, HPLC grade
Flow rate: 300 μL/min
Injection volume: 10 μl

HPLC analysis conditions used in QC:
HPLC system: Shimazu VP series
Column: C-18
Mobile phase: H₂O/CH₃CN/Formic acid (CH₃CN gradient from from 15% to 100%)
Detector: ELSD
Run time: 3.5 to 4.5 min.

Quality control data used in the acquisition of the compounds are shown below:

| | | | Confirmed | HPLC |
|---|---|---|---|---|
| Product | Compound # | Compound Name | Mass¹ | purity |
| A12B23C3 | 32 | 5-[4-Ethoxy-3-(8-isopropyl- | 461.20 | 72% |
| | | 3,5,5-trimethyl-5,6-dihydro- | | |
| | | naphthalen-2-yl)-benzylidene]- | | |
| | | thiazolidine-2,4-dione | | |
| A12B29C3 | 33 | 5-[4-Ethylamino-3-(8-isopropyl- | 460.22 | 100% |
| | | 3,5,5-trimethyl-5,6-dihydro- | | |
| | | naphthalen-2-yl)-benzylidene]- | | |
| | | thiazolidine-2,4-dione | | |
| A12B30C3 | 34 | 5-[4-Ethyl-3-(8-isopropyl-3,5,5- | 445.21 | 88% |
| | | trimethyl-5,6-dihydro- | | |
| | | naphthalen-2-yl)-benzylidene]- | | |
| | | thiazolidine-2,4-dione | | |
| A12B31C3 | 35 | 5-[4-Chloro-3-(8-isopropyl- | 451.14 | 84% |
| | | 3,5,5-trimethyl-5,6-dihydro- | | |
| | | naphthalen-2-yl)-benzylidene]- | | |
| | | thiazolidine-2,4-dione | | |
| A12B70C3 | 36 | 5-[3-Bromo-5-(8-isopropyl- | 495.09 | 100% |
| | | 3,5,5-trimethyl-5,6-dihydro- | | |
| | | naphthalen-2-yl)-benzylidene]- | | |
| | | thiazolidine-2,4-dione | | |
| A12B71C3 | 37 | 5-[4-(Ethyl-methyl-amino)-3-(8- | 474.23 | 100% |
| | | isopropyl-3,5,5-trimethyl-5,6- | | |
| | | dihydro-naphthalen-2-yl)- | | |
| | | benzylidene]-thiazolidine-2,4- | | |
| | | dione | | |
| A12B23C1 | 38 | 5-[4-Ethoxy-3-(8-isopropyl- | 477.18 | 68% |
| | | 3,5,5-trimethyl-5,6-dihydro- | | |
| | | naphthalen-2-yl)-benzylidene]-2- | | |
| | | thioxo-thiazolidin-4-one | | |
| A12B25C1 | 39 | 5-[4-Dimethylamino-3-(8- | 476.20 | 100% |
| | | isopropyl-3,5,5-trimethyl-5,6- | | |
| | | dihydro-naphthalen-2-yl)- | | |
| | | benzylidene]-2-thioxo- | | |
| | | thiazolidin-4-one | | |
| A12B29C1 | 40 | 5-[4-Ethylamino-3-(8-isopropyl- | 476.20 | 100% |
| | | 3,5,5-trimethyl-5,6-dihydro- | | |
| | | naphthalen-2-yl)-benzylidene]-2- | | |
| | | thioxo-thiazolidin-4-one | | |

¹Compound Mass confirmed by HPLC/MS of MH+ mass peak.

### Comparative Example 17

### 5-[3-(3,5,5,8,8-Pentamethyl-5,6,7,8-tetrahydro-naphthalen-2-yl)-4-trifluoromethoxy-benzylidene]-thiazolidine-2,4-dione, which can be referred to as “Compound 41”

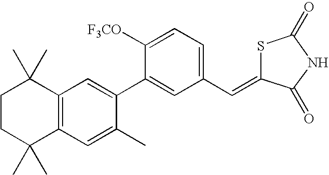 The synthesis of Compound 41 was disclosed in U.S. Pat. No. 6,515,003, issued Feb. 4, 2003, which is incorporated herein in its entirety by this reference.

### Example 18

### Differentiation of 3T3-L1 Pre-Adipocytes In An In Vitro Assay (See Results in FIG. 1a-b)

The following protocol was used to determine adipocyte differentiation activity of the compounds of the invention:

Mouse pre-adipocyte 3T3-L1 cells obtained from ATCC (American Tissue Culture Collection, MD) were initially grown in DME Dulbecco's modified Eagle's medium containing 4500 mg/L glucose; 4 mM L-glutamine; 10 U/ml Pen-G; 10 mcg/ml Streptomycin and 10% Bovine Calf Serum (CS) at 37° C. and 10% CO₂. Cells were plated in 96 well plates at a density of approximately 3,000 cells/well and grown to confluence (when cells use 100% of the available space on the well) in the same medium. Differentiation experiments were conducted two days after confluence in a differentiation medium (DM) consisting of DME Dulbecco's modified Eagle's medium containing 4500 mg/L glucose; 4 mM L-glutamine; 10 U/ml Pen-G; 10 mcg/ml Streptomycin and 10% Fetal Calf Serum (FCS) and 1 μg/mL of insulin. Cells were then treated with the test compound at a concentration of 10⁻¹⁰ to 10⁻⁶M, or with a control for fully-differentiated adipocytes, such as Dexamethasone/Insulin (2.5 μM; 10 μg/ml, respectively). Differentiation medium containing the compounds, with no further addition of insulin, was replaced every 2-3 days for a total of 7 days. Compound 41 was used as a standard for differention activity, and its ability to differentiate 3T3-L1 cells at 0.1 μM was taken as reference for 100% differentiation. Upon termination of the experiments the treated cells were washed once with PBS (Phosphate Buffer Saline, Irvine Scientific, Irvine, Calif.) and lysed in situ with 50 μL 10% Hecameg (Detergent, Calbiochem, San Diego). The cellular lysates were analyzed for their lipid content using the Triglyceride-GPO Trinder reagent from Sigma.

As shown in FIGS. 1a-b, many of compounds of the invention induce differenciation of 3T3-L1 cells.

### Example 19

### Oral Administration of Selected Compounds in the Treatment of Type 2 Diabetes in KKA^{y} Mice (FIG. 2a-d)

The procedure for this in-vivo assay for anti-diabetes activity was described in detail by Iwatsuka, et al. (1970 General Survey of Diabetic Features of Yellow KK Mice. Endocrinol. Japon. 17: 23-35, incorporated herein in its entirety by reference).

Experimental Procedures: Six to eight week-old male KKA^{Y} mice (obtained from Jackson Labs of Bar Harbor, Me.) were housed in a fixed 12-12-hr artificial light-dark cycle, and maintained on a standard rodent diet provided ad libitum. Animals were allowed two days to acclimate in this experimental environment prior to the initiation of the study.

Prior to initiation of treatment with the compounds of the invention, the animals were bled from the tail vein (100-200 μL of whole blood) and serum levels of glucose and triglycerides were measured in duplicate (Trinder kits; Sigma, St. Louis, Mo.). Based on these initial measures, animals were sorted into groups with approximately the same average serum glucose levels. Once sorted, the animals were housed one per cage and provided rodent diet ad libitum. Unless otherwise indicated, compounds were suspended in sesame oil, and administered by oral gavage once daily to animals in a volume of 3 ml/kg/dose.

Treatment Group A (n=5/group): (See Results in FIG. 2a)

1) KKA^{y} vehicle control (sesame oil)
2) Compound 1 (15 mg/kg)
   Treatment Group B (n=5/group): (See Results in FIG. 2b)
1) KKA^{y} vehicle control (sesame oil)
2) Compound 8 (3 mg/kg)
3) Compound 8 (10 mg/kg)
   Treatment Group C (n=5/group): (See Results in FIG. 2c)
1) KKA^{y} vehicle control (sesame oil)
2) Compound 9 (3 mg/kg)
3) Compound 9 (10 mg/kg)
   Treatment Group D (n=5/group): (See Results in FIG. 2d)
1) KKA^{y} vehicle control.(10% HPβCD)
2) Compound 12 (0.3 mg/kg)
3) Compound 12 (1 mg/kg)
4) Compound 12 (3 mg/kg)
5) Compound 12 (10 mg/kg)
   Compound 12 was dissolved in a 10% hydroxy propyl beta cyclodextrin solution, and administered to animals in a volume of 10 ml/kg/dose. (n=5/group): (See Results in FIG. 2d)

To monitor the effect of the tested compounds, animals were bled at the end of the dark cycle on days 3, 7, 10, or 14 of the treatment period. Serum glucose and triglyceride levels were measured in duplicate. The blood is kept at room temperature to allow coagulation, after which the serum is separated and assayed for glucose and triglyceride levels. As shown in FIGS. 2a-d all of the compounds tested reduced serum glucose and triglyceride levels, some with doses as low as 3 mg/kg when administered once a day.

### Example 20

### Cholesterol Efflux Assay from Macrophage Foam Cells as Induced by Compound 11(See Results in FIG. 3)

Cholesterol efflux from macrophage foam cells was assayed as described by Sparrow. et al, J. Biol. Chem., 2002, 277, 10021-10027, which is incorporated herein in its entirety by this reference. THP-1 cells obtained from ATCC (Manassas, VI), were cultured in RPMI medium (Sigma, St-Louis, Mo.), containing 10% fetal calf serum (Sigma, St-Louis, Mo.), 0.05 μM 2-mercaptoethanol, 1 mM sodium pyruvate, 2 mM L-glutamine, 100 units/ml penicillin, 0.1 μg/ml streptomycin and 0.25 μg/ml amphotericin B obtained from Sigma (St-Louis, Mo.). The THP-1 cells were differentiated into macrophages in 24 well tissue culture dishes at a density of 0.5 million cells/well by incubation in the same medium plus 100 nM tetradecanoyl phorbol acetate (Sigma, St-Louis, Mo.), for 3 days.

After differentiation into macrophages, the cells were tested for cholesterol efflux as induced by Compound 11 of the invention. Cells were labeled by incubation for 24 hr in fresh growth medium containing [3H]-cholesterol (10 μCi/ml) (PerkinElmer, Boston, Mass.), and 50 μg/ml acetylated-LDL (Frederick, Md.) and 1% Fetal bovine serum (Sigma, St-Louis, Mo.). Following labeling with [3H]-cholesterol, cells were washed, and incubated for an additional 24 hr in serum-free media containing 1 mg/ml bovine serum albumin (Sigma, St-Louis, Mo.), to allow for equilibration of [3H]-cholesterol with intracellular cholesterol. Cholesterol efflux was initiated by adding the 10 μg/ml ApoA-I (CalBiochem, La Jolla, Calif.), with or without Compound 2 (1 μM final concentration) in serum free media. Compound 11 was added to cultured cells from stock solution, and control cells received an equivalent amount of vehicle. After 24 hr, media were harvested and cells were dissolved in 1 mM HEPES, pH 7.5 containing 0.5% of a detergent Triton X-100 (Sigma, St-Louis, Mo.). Media were briefly centrifuged to remove non-adherent cells, and then aliquots of both the supernatant and the dissolved cells were counted by liquid scintillation spectrometry to determine radioactivity.

Cholesterol efflux is expressed as a percentage, calculated as
([3H]Cholesterol in medium)/([3H]Cholesterol in medium+[3H]cholesterol in cells)×100
As shown in FIG. 3, compound 11 increases cholesterol efflux from THP-1 cells as compared to non treated cells.

### Example 21

### Oral Administration of Selected Compounds in the Treatment of Diet-Induced Hypercholesterolemia in Wild Type Sprague Dawley Rats (See Results in FIGS. 4a-b)

Experimental Procedure: Six week-old male Sprague Dawley rats (obtained from Harlan of San Diego, Calif.) were housed in a fixed 12-12-hr artificial light-dark cycle, and maintained on a high cholesterol atherogenic diet (Paigen's Diet, obtained from Research Diet Inc. of New Brounswick, N.J.) was provided ad libitum. Animals were allowed six days to acclimate in this experimental environment prior to the initiation of the study.

Prior to initiation of treatment, the animals were bled from the tail vein (100-200 μL of whole blood) and serum levels of cholesterol were measured in duplicate (Cholesterol Infinity kits; Sigma, St. Louis, Mo.). Based on these initial measures, animals were sorted into groups with approximately the same average total cholesterol levels. Once sorted, the animals were housed three per cage and maintained on Paigen's diet ad libitum. All compounds to be tested were suspended in sesame oil and administered in a final volume of 3 ml/kg. Drug is administered by oral gavage once daily at the beginning of the artificial light cycle. To obtain a base line for lipid measurement, a control group maintained on standart rodent diet is included (lean control).

Treatment Group A (n=6/group): (See Results in FIG. 4A)

1) Lean control (Sesame Oil)
2) Control
3) Compound 1 (10 mg/kg)
   Treatment Group B (n=6/group): (See Results in FIG. 4B)
1) Lean control (Sesame Oil)
2) Control
3) Compound 11 (3 mg/kg)

To monitor the effect of the tested compounds, animals were bled from the tail vein at the end of the dark cycle on days 0 (for sorting) and day 5 of the treatment period. Fed serum cholesterol levels were measured in duplicate. The blood is kept at room temperature to allow coagulation, after which the serum is separated and assayed for total cholesterol (Infinity reagent, Sigma), HDL cholesterol (using HDL precipitating reagent and infinity reagent, Sigma) and LDL cholesterol (EzLDL kit, Sigma). As shown in FIGS. 4a-b, all compounds tested show significant reduction in total and LDL cholesterol levels and a significant increase in HDL cholesterol levels compared to high fat fed control animals.

### Example 22

### Downregulation of Cyclin D1 by Selected Compounds (See Results in FIG. 5)

Western Blot Assay for Cyclin D1 protein, Experimental Procedure: MCF-7 breast cancer cell obtained from ATCC (Manassas, Va.), were cultured in 100 mm culture plates in DMEM (Sigma, St-Louis, Mo.), containing 10% fetal calf serum (FBS) (Sigma, St-Louis, Mo.), 0.05 μM 2-mercaptoethanol, 2 mM L-glutamine, 100 units/ml penicillin, 0.1 μg/ml streptomycin and 0.25 μg/ml amphotericin B obtained from Sigma (St-Louis, Mo.). Cells were grown in a humidified incubator with 5% CO₂ at 37° C. When they reach 70% confluency, media was removed and replaced with fresh DMEM media containing 5% FBS and indicated concentration of Compounds. After 24 hr culture, the cells were harvested in phosphate buffer saline pH 7.0 (PBS) (Gibco, Rockville, Mass.), by scraping and pelleted by centrifugation at 500×g for 5 min at 4° C. The cells were homogenized in 150 μl of extraction buffer [10 mM Tris (pH 7.4), 300 mM NaCl, 1 mM EDTA, 10 mM MgCl₂, 2 mM DTT, 5 mM phenylmethysulfonyl fluoride, 10 μg/ml aprotinin, 10 μg/ml leupeptin, and 0.5% NP40, all reagent from Sigma, St-Louis, Mo. or Calbiochem San Diego, Calif.]. The homogenate was centrifuged at 14,000×g for 15 min, the supernatant was collected, and protein concentrations were measured using a commercial Bio-Rad assay (Biorad, Herculis, Calif.). 50 μg of whole-cell extract proteins were subjected to 10% SDS-PAGE (Invitrogen, Carlsbad, Calif.), transferred to polyvinylidene difluoride nitrocellulose membrane (Biorad, Herculis, Calif.), and probed with anti-cyclin D1 antibody (NeoMarkers, Fremont, Calif.). Immunoreactive proteins were visualized by enhanced chemiluminescence detection (Amersham, Piscataway, N.J.) and X-film.

As shown in FIG. 5 Cyclin D1 expression is decrease when the cells are treated with many of the compounds tested as compared to control (untreated cells).

### Example 23

### Oral Administration of Selected Compounds Slows the Progression of Mammary Tumors in Sprague Dawley Rats (See Results in FIG. 6)

Procedure: Five week-old female Sprague Dawley rats (Harlan) were housed in a fixed 12-12-hr artificial light-dark cycle, and maintained on a standard rodent diet provided ad libitum. Animals were allowed two days to acclimate in this experimental environment prior to the initiation of the study.

To induce mammary tumors, the female mice were injected intraperitoneally with the carcinogen n-nitroso-n-methylurea, in a single dose of 50 mg/kg in acidified normal saline (pH4 w/acetic acid) at a final volume of 10 mg/ml (5 ml/kg). After eight weeks, mammary tumors are detected, and the tumor bearing females are sorted into treatment groups. Once sorted, the animals were housed four per cage and provided rodent diet ad libitum. All animals are treated with test compound or a vehicle for four weeks, during which time, changes in tumor size are monitored. Tumors were classified as regressing, static or progressing.

Treatment groups (n=8/group):
1) Control (sesame oil)
2) Tamoxifen (800 μg/kg)
3) Compound 1 (10 mg/kg)
4) Compound 1 (20 mg/kg)
5) Compound 1 (50 mg/kg)
6) Compound 1 (100 mg/kg)
7) Compound 1 (20 mg/kg)+Tamox. (800 μg/kg)
8) Compound 9 (75 mg/kg)
9) Compound 11 (20 mg/kg)
10) Compound 11 (20 mg/kg)+Tamox. (800 μg/kg)
11) Compound 11 (50 mg/kg)

All of the compounds of the invention were suspended in sesame oil, and administered to animals in a volume of 3 ml/kg/dose by oral gavage, tamoxifen was dissolved in sesame oil and administered five days/week in a volume of 100 μL injected subcutaneously.

To monitor the effect of the tested compound, animals were examined for mammary tumors once every week. Tumors were classified into one of three categories, progressing, static or regressing. All of the compounds tested slowed the progression of mammary tumors compared to vehicle treated controls as shown in FIG. 6. Further, several compounds show some synergistic/additive effects with tamoxifen, as indicated by the improved efficacy profile in the combination treatment compared to treatment with tamoxifen alone.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An isochroman compound having the structure 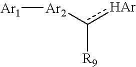
wherein
a) Ar₁ has the structure 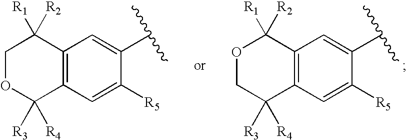
wherein R₁, R₂, R₃, and R₄ are independently selected from hydrogen, halogen, amino, and/or substituents comprising 1 to 4 carbon atoms selected from alkyl, haloalkyl, cyano, mono-substituted amino, di-substituted amino, alkoxy, haloalkoxy, carboalkoxy, acyl, alkylcarboxamido, dialkylcarboxamido, alkylamido, acyloxy; and R₅ is selected from hydrogen, a halogen, amino, —SH, or a radical comprising 1 to 4 carbon atoms selected from alkyl, mono-substituted amino, di-substituted amino, alkoxy, haloalkoxy, thioalkyl, or thioacyl;
b) AR₂ has the structure 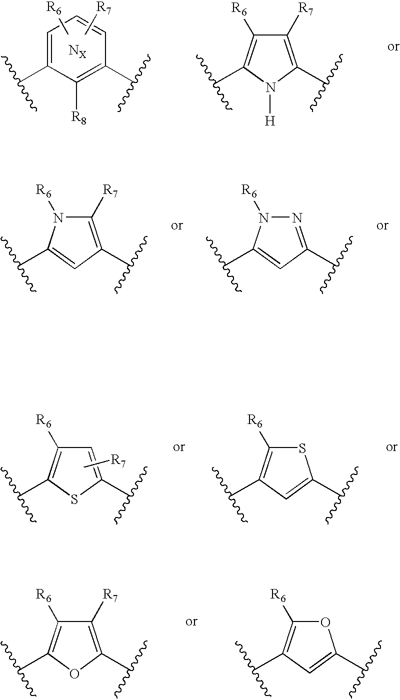
wherein X is an integer selected from 0, 1, or 2, and R₆, R₇ and R₈ are independently selected from hydrogen, halogen, amino, nitro, and/or substituents comprising 1 to 4 carbon atoms selected from alkyl, haloalkyl, cyano, mono-substituted amino, di-substituted amino, alkoxy, haloalkoxy, carboalkoxy, alkylcarboxamido, dialkylcarboxamido, alkylamido, acyloxy, —SH, thioalkyl, or thioacyl;
c) R₉ is hydrogen, hydroxy, or an alkyl radical comprising 1 to 4 carbon atoms;
d) 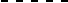
is either present or absent; and
e) HAr has the structure 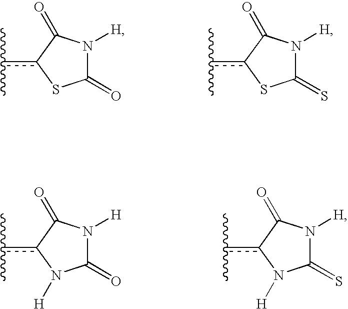
or a pharmaceutically acceptable salt thereof.

2. The compound of claim 1 wherein R₁, R₂, R₃, and R₄ are independently selected from hydrogen and alkyls comprising 1 to 4 carbon atoms; and R₅ is selected from hydrogen, fluorine, amino, —SH, methyl, ethyl, mono-methyl amino, dimethyl amino, methoxy, trifluoromethoxy, or thiomethyl.

3. The compound of claim 1 wherein R₁, R₂, R₃, and R₄ are methyl; and R₅ is selected from hydrogen, fluorine, amino, —SH, methyl, ethyl, mono-methyl amino, dimethyl amino, methoxy, trifluoromethoxy, or thiomethyl.

4. The compound of claim 1 wherein AR₂ has the structure

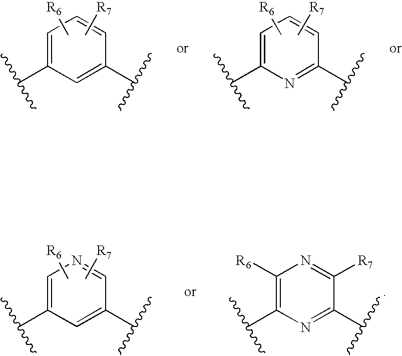

5. The compound of claim 1 wherein AR₂ has the structure 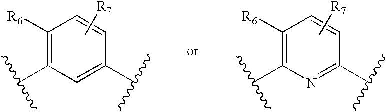
wherein R₆ is halo, methyl, ethyl, isopropyl, hydroxymethyl, hydroxyethyl, amino, methylamino, dimethylamino, hydroxyl, methoxy, or trifluoromethoxy.

6. The compound of claim 1 wherein AR₂ has the structure

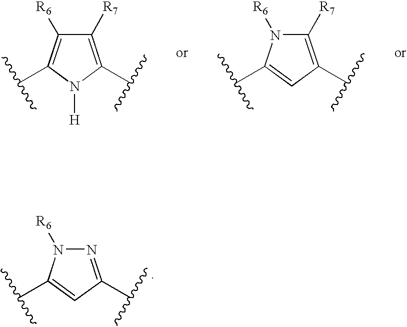

7. The compound of claim 1 wherein AR₂ has the structure 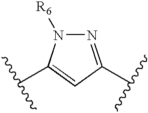
wherein R₆ is halo, methyl, ethyl, isopropyl, hydroxymethyl, hydroxyethyl, amino, methylamino, dimethylamino, hydroxyl, methoxy, or trifluoromethoxy.

8. The compound of claim 1 wherein R₉ is hydrogen.

9. The compound of claim 1 wherein - - - is present.

10. The compound of claim 1 wherein HAr has the structure

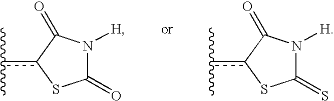

11. The compound of claim 1 having the formula
5-[2,5-Difluoro-4-methoxy-3-(1,1,4,4,7-pentamethyl-isochroman-6-yl)-benzylidene]-thiazolidine-2,4-dione;
5-[3-(1,1,4,4,7-Pentamethyl-isochroman-6-yl)-4-trifluoromethoxy-benzylidene]-thiazolidine-2,4-dione;
5-[4-Dimethylamino-3-(1,1,4,4,7-pentamethyl-isochroman-6-yl)-benzylidene]-thiazolidine-2,4-dione;
5-[3-(7-Chloro-1,1,4,4-tetramethyl-isochroman-6-yl)-4-trifluoromethoxy-benzylidene]-thiazolidine-2,4-dione
5-[2,5-Difluoro-4-methoxy-3-(1,1,4,4,7-pentamethyl-isochroman-6-yl)-benzylidene]-thiazolidine-2,4-dione;
5-[3-(1,1,4,4,7-Pentamethyl-isochroman-6-yl)-4-trifluoromethoxy-benzylidene]-thiazolidine-2,4-dione;
5-[4-Dimethylamino-3-(1,1,4,4,7-pentamethyl-isochroman-6-yl)-benzylidene]-thiazolidine-2,4-dione; and
5-[3-(7-Chloro-1,1,4,4-tetramethyl-isochroman-6-yl)-4-trifluoromethoxy-benzylidene]-thiazolidine-2,4-dione.

12. A pharmaceutical composition comprising one or more of the compounds of claim 1 or pharmaceutically acceptable salts or prodrugs thereof, and one or more pharmaceutically acceptable carriers.

13. A method for the treatment of a disease of uncontrolled cellular proliferation comprising administering to a mammal diagnosed as having a disease of uncontrolled cellular proliferation one or more compounds of claim 1 or pharmaceutically acceptable salts or prodrugs thereof, or a pharmaceutical composition thereof, in an amount effective to treat the disease of uncontrolled cellular proliferation.

14. The method of claim 13 wherein the disease of uncontrolled proliferation is a carcinoma, lymphoma, leukemia, or sarcoma.

15. The method of claim 13 wherein the disease of uncontrolled proliferation is a cancer.

16. The method of claim 15 wherein the cancer is lymphoma, Hodgkin's Disease, myeloid leukemia, bladder cancer, brain cancer, head and neck cancer, kidney cancer, lung cancers such as small cell lung cancer and non-small cell lung cancer, myeloma, neuroblastoma/glioblastoma, ovarian cancer, pancreatic cancer, prostate cancer, skin cancer, liver cancer, melanoma, colon cancer, cervical carcinoma, breast cancer, or epithelial cancer.

17. The method of claim 15 that additionally comprises administration of one or more additional therapeutic agents effective for the treatment of the cancer.

18. A method of modulating lipid metabolism, carbohydrate metabolism, or lipid and carbohydrate metabolism comprising administering to a mammal diagnosed as needing such modulation one or more of the compounds of claim 1 or pharmaceutically acceptable salts or prodrugs thereof, in an amount effective to induce such modulation.

19. A method of treating hypercholesterolimia comprising administering to a mammal diagnosed as needing such treatment one or more compounds of claim 1 or pharmaceutically acceptable salts or prodrugs thereof, in an amount effective to treat the hypercholesterolimia.

20. The method of claim 19, wherein the one or more compounds is applied in an amount effective to decrease serum cholesterol levels by at least about 5%.

21. A method of treating dyslipidemia comprising administering to a mammal diagnosed as needing such treatment one or more compounds of claim 1 or pharmaceutically acceptable salts or prodrugs thereof, in an amount effective to decrease serum triglyceride levels.

22. The method of claim 21, wherein the one or more compounds are applied in an amount effective to decrease serum triglyceride levels by at least about 5%.

23. A method of treating Type 2 Diabetes comprising administering to a mammal diagnosed as needing such treatment one or more compounds of claim 1 or pharmaceutically acceptable salts or prodrugs thereof, in an amount effective to treat the Type 2 Diabetes.

24. The method of claim 23, wherein the compound is applied in an amount effective to to decrease the serum glucose levels in the mammal by at least about 5%.

25. The method of claim 24 wherein the administration is also effective to decrease serum triglyceride levels in the mammal by at least about 5%.

26. The method of claim 23 wherein the mammal is a human.

27. A dihydronaphthalene compound having the structure 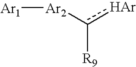
wherein
a) Ar₁ has the structure 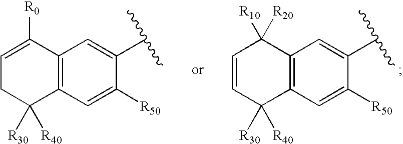
wherein R₀ is selected from hydrogen, a halogen, an aryl or heteroaryl comprising 1 to 8 carbon atoms, and radicals comprising 1 to 4 carbon atoms selected from alkyl, haloalkyl, di-substituted amino, alkoxy, haloalkoxy, or acyloxy; and R₁₀, R₂₀, R₃₀, and R₄₀. are independently selected from substituents comprising 1 to 4 carbon atoms selected from alkyl, haloalkyl, cyano, amino, mono-substituted amino, di-substituted amino, alkoxy, haloalkoxy, carboalkoxy, alkylcarboxamido, dialkylcarboxamido, alkylamido, or acyloxy, and R₅₀ is selected from hydrogen, a halogen, amino, —SH, or a radical comprising 1 to 4 carbon atoms selected from alkyl, mono-substituted amino, di-substituted amino, alkoxy, haloalkoxy, thioalkyl, or thioacyl;
b) AR₂ has the structure 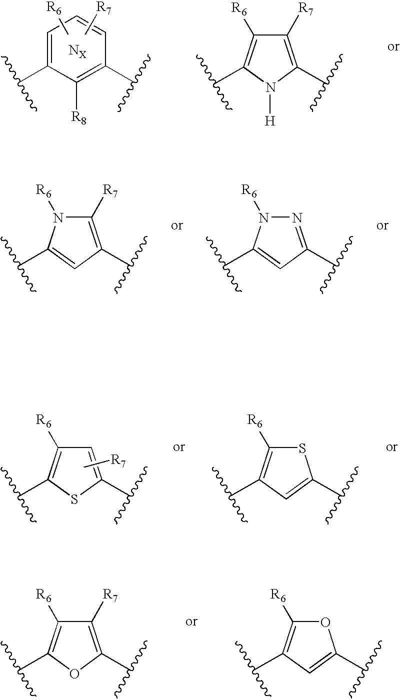
wherein X is an integer selected from 0, 1, or 2, and R₆, R₇ and R₈ are independently selected from hydrogen, halogen, amino, nitro, and substituents comprising 1 to 4 carbon atoms selected from alkyl, haloalkyl, cyano, mono-substituted amino, di-substituted amino, alkoxy, haloalkoxy, carboalkoxy, alkylcarboxamido, dialkylcarboxamido, alkylamido, acyloxy, —SH, thioalkyl, or thioacyl;
c) R₉ is hydrogen, hydroxy, or an alkyl radical comprising 1 to 4 carbon atoms;
d) 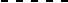
is either present or absent; and
e) HAr has the structure 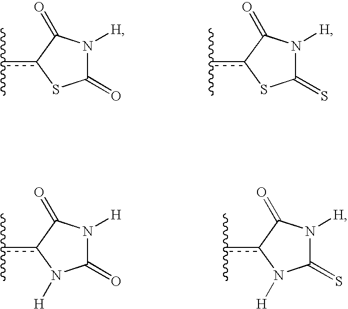
or a pharmaceutically acceptable salt thereof.

28. The compound of claim 27 wherein R₁₀, R₂₀, R₃₀, and R₄₀ are independently selected from hydrogen, and alkyls comprising 1 to 4 carbon atoms; and R₀ is hydrogen, fluorine, phenyl, fluorophenyl, benzyl, hydroxyphenyl, pyridyl, methyl, ethyl, propyl, isopropyl, trifluoromethyl, dimethyl amino, methoxy, or trifluoromethoxy.

29. The compound of claim 27 wherein R₁₀, R₂₀, R₃₀, and R₄₀ are methyl; and R₅₀ is selected from hydrogen, fluorine, amino, —SH, methyl, ethyl, mono-methyl amino, dimethyl amino, methoxy, trifluoromethoxy, and thiomethyl, and R₀ is hydrogen, fluorine, phenyl, fluorophenyl, benzyl, hydroxyphenyl, pyridyl, methyl, ethyl, propyl, isopropyl, trifluoromethyl, dimethyl amino, methoxy, or trifluoromethoxy.

30. The compound of claim 27 wherein AR₂ has the structure

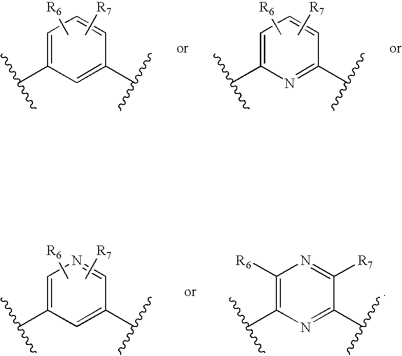

31. The compound of claim 27 wherein AR₂ has the structure 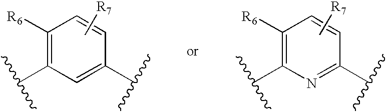
wherein R₆ is halo, methyl, ethyl, isopropyl, hydroxymethyl, hydroxyethyl, amino, methylamino, dimethylamino, hydroxyl, methoxy, or trifluoromethoxy.

32. The compound of claim 27 wherein AR₂ has the structure

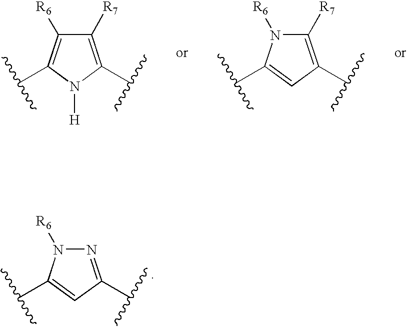

33. The compound of claim 27 wherein AR₂ has the structure 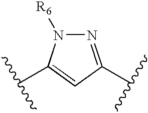
wherein R₆ is halo, methyl, ethyl, isopropyl, hydroxymethyl, hydroxyethyl, amino, methylamino, dimethylamino, hydroxyl, methoxy, or trifluoromethoxy.

34. The compound of claim 27 wherein R₉ is hydrogen.

35. The compound of claim 27 wherein 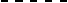
is present.

36. The compound of claim 27 wherein HAr has the structure

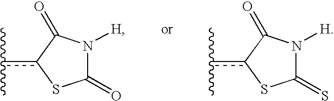

37. The compound of claim 27 having the formula
5-[4-Dimethylamino-3-(8-isopropyl-3,5,5-trimethyl-5,6-dihydro-naphthalen-2-yl)-benzylidene]-thiazolidine-2,4-dione;
5-[2,5-Difluoro-3-(8-isopropyl-3,5,5-trimethyl-5,6-dihydro-naphthalen-2-yl)-4-methoxybenzylidene]-thiazolidine-2,4-dione;
5-[4-Trifluoromethoxy-3-(3,5,5-trimethyl-8-phenyl-5,6-dihydro-naphthalen-2-yl)-benzylidene]-thiazolidine-2,4-dione;
5-[4-Trifluoromethoxy-3-(3,5,5-trimethyl-8-thiophen-2-yl-5,6-dihydro-naphthalen-2-yl)-benzylidene]-thiazolidine-2,4-dione;
5-[4-Trifluoromethoxy-3-(3,5,5-trimethyl-8-thiophen-3-yl-5,6-dihydro-naphthalen-2-yl)-benzylidene]-thiazolidine-2,4-dione;
5-[4-Trifluoromethoxy-3-(3,5,5-trimethyl-8-thiophen-2-yl-5,6-dihydro-naphthalen-2-yl)-benzylidene]-thiazolidine-2,4-dione;
5-[2,5-Difluoro-4-methoxy-3-(3,5,5,8,8-pentamethyl-5,8-dihydro-naphthalen-2-yl)-benzylidene]-thiazolidine-2,4-dione;
5-[3-(3,5,5,8,8-Pentamethyl-5,8-dihydro-naphthalen-2-yl)-4-trifluoromethoxy-benzylidene]-thiazolidine-2,4-dione;
5-[4-Dimethylamino-3-(3,5,5,8,8-pentamethyl-5,8-dihydro-naphthalen-2-yl)-benzylidene]-thiazolidine-2,4-dione;
5-[4-Ethoxy-3-(8-isopropyl-3,5,5-trimethyl-5,6-dihydro-naphthalen-2-yl)-benzylidene]-thiazolidine-2,4-dione;
5-[4-Ethylamino-3-(8-isopropyl-3,5,5-trimethyl-5,6-dihydro-naphthalen-2-yl)-benzylidene]-thiazolidine-2,4-dione;
5-[4-Ethyl-3-(8-isopropyl-3,5,5-trimethyl-5,6-dihydro-naphthalen-2-yl)-benzylidene]-thiazolidine-2,4-dione;
5-[4-Chloro-3-(8-isopropyl-3,5,5-trimethyl-5,6-dihydro-naphthalen-2-yl)-benzylidene]-thiazolidine-2,4-dione;
5-[3-Bromo-5-(8-isopropyl-3,5,5-trimethyl-5,6-dihydro-naphthalen-2-yl)-benzylidene]-thiazolidine-2,4-dione;
-[4-(Ethyl-methyl-amino)-3-(8-isopropyl-3,5,5-trimethyl-5,6-dihydro-naphthalen-2-yl)-benzylidene]-thiazolidine-2,4-dione;
5-[4-Ethoxy-3-(8-isopropyl-3,5,5-trimethyl-5,6-dihydro-naphthalen-2-yl)-benzylidene]-2-thioxo-thiazolidin-4-one;
5-[4-Dimethylamino-3-(8-isopropyl-3,5,5-trimethyl-5,6-dihydro-naphthalen-2-yl)-benzylidene]-2-thioxo-thiazolidin-4-one; or
5-[4-Ethylamino-3-(8-isopropyl-3,5,5-trimethyl-5,6-dihydro-naphthalen-2-yl)-benzylidene]-2-thioxo-thiazolidin-4-one.

38. The compound of claim 27 having the formula
5[3-(8-Isopropyl-3,5,5-trimethyl-5,6-dihydro-naphthalen-2-yl)-4-trifluoromethoxy-benzylidene]-thiazolidine-2,4-dione.

39. A pharmaceutical composition comprising one or more of the compounds of claim 27 or pharmaceutically acceptable salts or prodrugs thereof, and one or more pharmaceutically acceptable carriers.

40. A method for the treatment of a disease of uncontrolled cellular proliferation comprising administering to a mammal diagnosed as having a disease of uncontrolled cellular proliferation one or more compounds of claim 27 or pharmaceutically acceptable salts or prodrugs thereof, or a pharmaceutical composition thereof, in an amount effective to treat the disease of uncontrolled cellular proliferation.

41. The method of claim 40 wherein the disease of uncontrolled proliferation is a carcinoma, lymphoma, leukemia, or sarcoma.

42. The method of claim 40 wherein the disease of uncontrolled proliferation is a cancer.

43. The method of claim 42 wherein the cancer is lymphoma, Hodgkin's Disease, myeloid leukemia, bladder cancer, brain cancer, head and neck cancer, kidney cancer, lung cancers such as small cell lung cancer and non-small cell lung cancer, myeloma, neuroblastoma/glioblastoma, ovarian cancer, pancreatic cancer, prostate cancer, skin cancer, liver cancer, melanoma, colon cancer, cervical carcinoma, breast cancer, or epithelial cancer.

44. The method of claim 43 that additionally comprises administration of one or more additional therapeutic agents effective for the treatment of the cancer.

45. A method of modulating lipid metabolism, carbohydrate metabolism, or lipid and carbohydrate metabolism comprising administering to a mammal diagnosed as needing such modulation one or more of the compounds of claim 27 or pharmaceutically acceptable salts or prodrugs thereof, in an amount effective to induce such modulation.

46. A method of treating hypercholesterolimia comprising administering to a mammal diagnosed as needing such treatment one or more compounds of claim 27 or pharmaceutically acceptable salts or prodrugs thereof, in an amount effective to treat the hypercholesterolimia.

47. The method of claim 46, wherein the one or more compounds is applied in an amount effective to decrease serum cholesterol levels by at least about 5%.

48. A method of treating dyslipidemia comprising administering to a mammal diagnosed as needing such treatment one or more compounds of claim 27 or pharmaceutically acceptable salts or prodrugs thereof, in an amount effective to decrease serum triglyceride levels.

49. The method of claim 48, wherein the one or more compounds are applied in an amount effective to decrease serum triglyceride levels by at least about 5%.

50. A method of treating Type 2 Diabetes comprising administering to a mammal diagnosed as needing such treatment one or more compounds of claim 27 or pharmaceutically acceptable salts or prodrugs thereof, in an amount effective to treat the Type 2 Diabetes.

51. The method of claim 50, wherein the compound is applied in an amount effective to to decrease the serum glucose levels in the mammal by at least about 5%.

52. The method of claim 50 wherein the administration is also effective to decrease serum triglyceride levels in the mammal by at least about 5%.

53. The method of claim 50 wherein the mammal is a human.
